(19) United States
(12) Patent Application Publication
(10) Pub. No.: US 2026/0207767 A1
(43) Pub. Date: Jul. 23, 2026

(54) IL-2 FUSION PROTEIN

(71) Applicant: ANAVEON AG, Basel (CH)

(72) Inventors: Christoph HUBER, Basel (CH); Andreas KATOPODIS, Basel (CH); Patrizia MURER

(73) Assignee: ANAVEON AG, Basel (CH)

(21) Appl. No.: 19/142,206

(22) PCT Filed: Dec. 21, 2023
(86) PCT No.: PCT/EP2023/087494

§ 371 Date: Jun. 22, 2025

(30) Foreign Application Priority Data
Dec. 22, 2022 (EP) 22216262.0
Mar. 13, 2023 (EP) 23161632.7
Mar. 16, 2023 (EP) 23162288.7
Jul. 13, 2023 (EP) 23185440.7
Sep. 26, 2023 (EP) 23199835.2

(51) Int. Cl. A61K 47/68; C07K 16/24; C07K 16/28

(52) U.S. Cl. CPC A61K 47/6879; A61K 47/6813; C07K 16/246; C07K 16/2818

(57) ABSTRACT

The invention relates to PD-1:PD-L1 non-blocking immunoconjugates comprising an IL-2 polypeptide providing a biased signal to the dimeric IL-2 receptor, and a PD-1 targeting domain, and their use for treating cancer.

### FIELD

The present invention relates to an IL-2 fusion protein designed to target exhausted T cells expressing PD-1. The invention further relates to pharmaceutical compositions comprising the IL-2 fusion protein, and methods of treating cancer patients with same, optionally in combination with PD-1 or PD-L1 antagonist antibodies.

### BACKGROUND

Recombinant IL-2 (aldesleukin) is a promising immunotherapy that activates IL-2 receptors expressed on effector T cell subsets including CD8+ T cells, and NK cells. In its native form IL-2 is a 15 kDa protein that can signal through the high affinity trimeric receptor IL-2R alpha/beta/gamma [composed of the IL-2R alpha chain (CD25), the IL-2R beta chain (CD122) and the IL-2R gamma chain (CD132)] or through the medium affinity dimeric receptor IL-2R beta/gamma. The CD122 and CD132 chains are capable of intracellular signaling via coupling to the JAK and PI3K families of kinases while CD25 does not appear to participate in signaling but increases the receptor affinity for IL-2 by 10- to 100-fold. Regulatory T cells (Tregs) and lung endothelial cells express CD25 and predominantly utilize the high affinity trimeric receptor while CD8+ T cells and NK cells (effector cells) do not express CD25 under steady state and display the dimeric receptor on their cell surface. This receptor expression profile limits the potential antitumor responses generated by treatment with aldesleukin, as immunosuppressive Tregs rather than CD8+ T cells and NK cells are preferentially stimulated, and lung endothelial cell activation through IL-2R contributes to the strong, IL-2 associated, side-effects such as hypotension and vascular leak syndrome.

One way to improve the efficacy of aldesleukin has been to develop molecules with biased signaling towards the medium affinity IL-2 receptors expressed by effector cells and limited activation of the high affinity IL-2 receptor expressed by Tregs which may suppress anti-tumor immunity (Noverwijk W. W. et al. 2021, Ann. Rev. Med. 72:281). The anti-tumor activity of such receptor-biased IL-2 molecules can be further improved by targeting the molecule to the tumor or to the tumor microenvironment, for example, by fusing the cytokine to antibodies binding tumor antigens. These molecules present IL-2 across cells and are often called trans-signaling IL-2 bispecifics. IL-2 constructs targeted to tumor tissues were shown to have a synergistic efficacy when used to treat cancer models in combination with a checkpoint inhibition antibody targeting PD-L1 (Klein C. 2017 Oncoimmunology 6:e1277306). Alternatively, receptor-biased IL-2 molecules can be targeted to CD8 T cells directly by using targeting antibodies binding to CD8, PD-1 or other antigens expressed on the surface of CD8 T cells. Because these constructs present IL-2 from the same T cell surface, they are often called cis-signaling IL-2 bispecifics. Such cis-signaling fusion proteins have been generated using anti-PD-1/IL-2 bispecifics, (WO2018184964A1, Deak L. C. et al. 2022, Nature 610:161) and were shown to have superior efficacy in mouse pre-clinical models. In all the known anti-PD-1/IL-2 constructs, the targeting arm consists of blocking anti-PD-1 antibodies (blocking PD-1/PD-1L interactions). Such a targeting arm has the potential advantage of relieving PD-L1 induced T cell suppression and delivering a proliferative IL-2 signal to the same effector cells. There is, however, an inherent difficulty of pharmacologically administering a blocking therapeutic (requiring full coverage of the target) and an agonistic therapeutic (requiring agonistic dosing and frequency). In the specific case of PD-1 and IL-2 therapeutics, anti-PD-1 antibodies are administered in mg/kg doses to ensure full target coverage, but IL-2 principles are administered in microgram/kg doses to avoid overstimulation and on-target toxicity. The discrepancy may be addressed by mutating the IL-2 portion of the molecule to reduce affinity to its receptor and thereby allow higher doses of the bispecific. Mutations to the IL-2 structure, however, may result in anti-drug antibodies which can render the therapy ineffective or toxic. Furthermore, in patients receiving PD-1 check point inhibitor (CPi) therapy, the epitope targeted by the PD-1/IL-2 fusion protein is already occupied by the CPi thus preventing binding of the fusion protein to the targeted cells.

Based on the above-mentioned state of the art, the objective of the present invention is to provide an IL-2 molecule with improved therapeutic characteristics. This objective is attained by the subject-matter of the independent claims of the present specification, with further advantageous embodiments described in the dependent claims, examples, figures and general description of this specification.

### SUMMARY OF THE INVENTION

The inventors sought to develop a PD-1 targeted therapeutic IL-2 molecule which does not inhibit the binding or antagonist action of conventional PD-1, or PD-L1 targeted checkpoint inhibitor antibodies. The inventors have generated non-blocking anti-PD-1 antibodies for targeting IL-2 to PD-1 expressing cells. The anti-PD-1 binding domains of non-blocking antibodies are used as a targeting moiety in an immunoconjugate further comprising an IL-2 polypeptide, delivering IL-2 signal to the appropriate exhausted CD8 T cells, and allowing optimal combination with existing anti-PD1 therapeutics. The resulting compound provides highly targeted IL-2 signaling to exhausted T cells, and may be combined more flexibly with existing CPi treatment regimens targeting the PD-1/PD-L1 signaling axis. This results in an immunoconjugate compound that effectively delivers IL-2 stimulation, while allowing modulated dosing of potent active agents to achieve safe and efficacious treatment of cancer.

One aspect of the invention is an IL-2 fusion protein immunoconjugate comprising an anti-PD-1 binding domain, and an anti-IL-2 antigen binding domain. Said anti-PD-1 binding domain comprises a heavy chain polypeptide and a light chain polypeptide derived from an anti-PD-1 antibody (an antibody capable of binding specifically to the extracellular domain of the human PD-1 protein). Said anti-IL-2 binding domain comprises a heavy chain polypeptide and a light chain polypeptide derived from an anti-IL-2 antibody (an antibody capable of binding specifically to the human IL-2 protein). One of the antibody variable domains within the heavy chain, or the light chain of the anti-IL-2 antigen binding domain is joined, optionally by means of one or two peptide linkers, to an IL-2 polypeptide.

In particular embodiments, the IL-2 is human IL-2, an artificial IL-2 variant polypeptide such as Proleukin, or an IL-2 mutein polypeptide. In certain embodiments, the IL-2 polypeptide is a circularly permuted IL-2 polypeptide (IL2CP), characterized by rearranged amino acid residues relative to the human IL-2 sequence. Together, the anti-IL-2 binding domain joined to the IL-2 polypeptide delivers biased IL-2 signaling to the dimeric IL-2 receptor expressed on target cells.

In particular embodiments, the anti-PD-1 binding domain incorporated into the immunoconjugate according to the invention is derived from a non-blocking PD-1 antibody. In other words, binding of the immunoconjugate to a cell expressing PD-1 does not significantly inhibit binding of PD-1 to its natural ligand PD-L1, or PD-1 antagonist compounds, such as the antibodies nivolumab, or pembrolizumab. This feature allows the immunoconjugate according to the invention, to be used as a medicament in combination with CPi compounds which inhibit checkpoint blockade by binding to either PD-1, or PD-L1.

In particular embodiments of the immunoconjugate according to the invention, the IL-2 polypeptide is an IL2CP embedded within the variable domain of the heavy or light chain of the anti-IL-2 binding domain, to provide a single fusion polypeptide. In alternative embodiments, the IL-2 polypeptide is joined to the N′ terminal region of the variable domain of either the heavy, or light chain of the anti-IL-2 binding domain by means of a peptide linker, to provide a single fusion polypeptide.

In certain embodiments of the immunoconjugate according to the invention, it comprises an Fc portion, particularly an IgG isotype Fc portion. In particular embodiments, the Fc portion is characterized by amino acid modifications to enhance correct heavy chain pairing, and/or to optimize levels of Fc effector functions.

In further particular embodiments, the immunoconjugate according to the invention is a heterotetrameric IgG (for example, a Crossmab, or kappa/lambda format) wherein one antibody arm comprises the anti-PD-1 binding domain, and one antibody arm comprises the anti-IL-2 antibody binding domain joined to an IL-2 polypeptide.

In certain embodiments, the immunoconjugate is an anti-PD-1 IgG wherein both antibody arms (each arm comprising a heavy chain polypeptide associated with a light chain polypeptide) are characterized by an anti-PD-1 binding domain. In such embodiments, IL-2 signaling is provided by fusion of an anti-IL-2 antibody binding domain in the format of a scFv, said scFv comprising an integrated, or linked IL-2 polypeptide. In particular embodiments of the immunoconjugate, the C′ terminal residue of the anti-IL-2 binding domain scFv is joined the N-terminal region of the anti-PD-1 antibody.

Further aspects of the invention relate to a pharmaceutical composition comprising the immunoconjugate, a nucleic acid encoding the immunoconjugate, or a host cell engineered to comprise said nucleic acid. The invention further encompasses the immunoconjugate according to the invention for use in treating cancer, and a method of treating a cancer patient by administering a therapeutically effective dose of the immunoconjugate according to the invention.

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising”, “having”, “containing”, and “including”, and other similar forms, and grammatical equivalents thereof, as used herein, are intended to be equivalent in meaning and to be open-ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictates otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a”, “or” and “the” include plural referents unless the context clearly dictates otherwise.

“And/or” where used herein is to be taken as specific recitation of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art (e.g., in cell culture, molecular genetics, nucleic acid chemistry, hybridization techniques and biochemistry, organic synthesis). Standard techniques are used for molecular, genetic, and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

The term PD-1 in the context of the present specification relates to the human PD-1 protein, encoded by the PDCD1 gene, also sometimes referred to as CD279 (Uniprot Q15116).

The term PD-L1 in the context of the present specification relates to the human PD-L1 protein, encoded by the gene CD274, also sometimes referred to as CD274 (Uniprot Q9NZQ7).

The term immunoconjugate in the context of the present specification refers to a recombinant polypeptide molecule comprising at least one anti-PD-1 antibody binding domain as disclosed herein, covalently linked to at least one additional IL-2-signalling moiety. The IL-2-signalling moiety comprises both an IL-2 polypeptide (for example, human IL-2, or an IL-2CP), linked, optionally by means of one or more short peptide linkers, to the antigen-binding domain of an anti-IL-2-specific antibody. The IL-2 portion of the immunoconjugate is capable of binding to an IL-2 receptor expressed on the cell surface of an immune cell, and initiating a downstream effect. In other words, the anti-PD-1 binding domain of the immunoconjugate acts as a targeting domain, and binding of said second IL-2-containing moiety to the cell initiates a downstream function in a target immune cell. IL-2-signalling moieties provided herein display preferential biased signaling through the dimeric IL-2 receptor.

The term IL-2 in the context of the present specification relates to human IL-2, and functional variants thereof, such as the wildtype human amino acid sequence (Uniprot P60568), and variant proteins listed in Table 2.

The term IL2CP, or IL2-CP in the context of the present specification relates to a circularly permuted IL-2 polypeptide is created by “opening” the IL-2 polypeptide sequence, to create a new N′ terminus and C′ terminus, and fusing natural N′ and C′ terminal amino acid residues. This generates a reordered IL2CP polypeptide retaining important tertiary structures of the cytokine that allow signaling through dedicated receptors.

The term dimeric IL-2 receptor in the context of the present specification relates to the heterodimer receptor comprising the two receptor chains CD122 and CD132.

Any patent document cited herein shall be deemed incorporated by reference herein in its entirety.

### Sequences

Sequences similar or homologous (e.g., at least about 70% sequence identity) to the sequences disclosed herein are also part of the invention. In some embodiments, the sequence identity at the amino acid level can be about 80%, 85%, 90%, 91%, 92%, 93%, 94%, 95%, 96%, 97%, 98%, 99% or higher. At the nucleic acid level, the sequence identity can be about 70%, 75%, 80%, 85%, 90%, 91%, 92%, 93%, 94%, 95%, 96%, 97%, 98%, 99% or higher. Alternatively, substantial identity exists when the nucleic acid segments will hybridize under selective hybridization conditions (e.g., very high stringency hybridization conditions), to the complement of the strand. The nucleic acids may be present in whole cells, in a cell lysate, or in a partially purified or substantially pure form.

In the context of the present specification, the terms sequence identity and percentage of sequence identity refer to a single quantitative parameter representing the result of a sequence comparison determined by comparing two aligned sequences position by position. Methods for alignment of sequences for comparison are well-known in the art. Alignment of sequences for comparison may be conducted by the local homology algorithm of Smith and Waterman, Adv. Appl. Math. 2:482 (1981), by the global alignment algorithm of Needleman and Wunsch, J. Mol. Biol. 48:443 (1970), by the search for similarity method of Pearson and Lipman, Proc. Nat. Acad. Sci. 85:2444 (1988) or by computerized implementations of these algorithms, including, but not limited to: CLUSTAL, GAP, BESTFIT, BLAST, FASTA and TFASTA. Software for performing BLAST analyses is publicly available, e.g., through the National Center for Biotechnology-Information (http://blast.ncbi.nlm.nih.gov/).

One example for comparison of amino acid sequences is the BLASTP algorithm that uses the default settings: Expect threshold: 10; Word size: 3; Max matches in a query range: 0; Matrix: BLOSUM62; Gap Costs: Existence 11, Extension 1; Compositional adjustments: Conditional compositional score matrix adjustment. One such example for comparison of nucleic acid sequences is the BLASTN algorithm that uses the default settings: Expect threshold: 10; Word size: 28; Max matches in a query range: 0; Match/Mismatch Scores: 1.-2; Gap costs: Linear. Unless stated otherwise, sequence identity values provided herein refer to the value obtained using the BLAST suite of programs (Altschul et al., J. Mol. Biol. 215:403-410 (1990)) using the above identified default parameters for protein and nucleic acid comparison, respectively.

Reference to identical sequences without specification of a percentage value implies 100% identical sequences (i.e. the same sequence).

### General Biochemistry: Peptides, Amino Acid Sequences

The term polypeptide in the context of the present specification relates to a molecule consisting of 50 or more amino acids that form a linear chain wherein the amino acids are connected by peptide bonds. The amino acid sequence of a polypeptide may represent the amino acid sequence of a whole (as found physiologically) protein or fragments thereof. The term “polypeptides” and “protein” are used interchangeably herein and include proteins and fragments thereof. Polypeptides are disclosed herein as amino acid residue sequences.

The term peptide in the context of the present specification relates to a molecule consisting of up to 50 amino acids, in particular 8 to 30 amino acids, more particularly 8 to 15 amino acids, that form a linear chain wherein the amino acids are connected by peptide bonds.

Amino acid residue sequences are given from amino to carboxyl terminus. Capital letters for sequence positions refer to L-amino acids in the one-letter code (Stryer, Biochemistry, 3^{rd} ed. p. 21). Lower case letters for amino acid sequence positions refer to the corresponding D- or (2R)-amino acids. Sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (lie, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

The term variant refers to a polypeptide that differs from a reference polypeptide, but retains essential properties. A typical variant of a polypeptide differs in its primary amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

In the context of the present specification, the term dimer refers to a unit consisting of two subunits. In the context of the present specification, the term heterodimer refers to a dimer comprised of two subunits that are not identical.

In the context of the present specification, the term amino acid linker or peptide linker refers to a polypeptide of variable length that is used to connect two polypeptides in order to generate a single chain polypeptide. Exemplary embodiments of linkers useful for practicing the invention specified herein are oligopeptide chains consisting of 1, 2, 3, 4, 5, 10, 20, 30, 40 or 50 amino acids.

There is no constraint on the amino acid composition of the linker. In certain embodiments, the linker consists of amino acids selected from the group of G S, A and D. An important characteristic of the conjugate peptide linkers as specified herein are low immunogenicity, and a peptide length that allows the domains which are joined by the linker, to interact to form a functional entity as disclosed herein. In particular desirable embodiments of the domain peptide linkers specified above, the sequences are primarily made up of stretches of small, polar amino acids such as glycine (G) and serine (S).

In certain embodiments peptide linker is ≥15 amino acids in length, particularly 15 to 30 amino acids in length wherein the amino acids are selected from G S, A and D.

A non-limiting example of an amino acid linker is a monomer or di-, tri- or tetramer of a peptide motif composed of three or four glycine and one serine.

Any embodiments relating peptide linkers as disclosed herein, encompass structures in which amino acids with similar characteristics are exchanged, for example, the amino acids V, L, I, P, S, C, or M may replace G, S, or S, and D may be replaced by E.

Particular non-limiting examples of linkers are provided by SEQ ID NO 014, 015, 016, 017, 018, 019, 020, 021, 022, 023, and 024.

### General Molecular Biology: Nucleic Acid Sequences, Expression

The term recombinant in the context of the present specification relates to a nucleic acid, which is the product of one or several steps of cloning, restriction and/or ligation and which is different from the naturally occurring nucleic acid. A recombinant virus particle comprises a recombinant nucleic acid.

The terms gene expression or expression, or alternatively the term gene product, may refer to either of, or both of, the processes—and products thereof—of generation of nucleic acids (RNA) or the generation of a peptide or polypeptide, also referred to transcription and translation, respectively, or any of the intermediate processes that regulate the processing of genetic information to yield polypeptide products. The term gene expression may also be applied to the transcription and processing of an RNA gene product, for example a regulatory RNA or a structural (e.g. ribosomal) RNA. If an expressed polynucleotide is derived from genomic DNA, expression may include splicing of the mRNA in a eukaryotic cell. Expression may be assayed both on the level of transcription and translation, in other words mRNA and/or protein product.

The term nucleic acid expression vector in the context of the present specification relates to a plasmid, a viral genome or an RNA, which is used to transfect (in case of a plasmid or an RNA) or transduce (in case of a viral genome) a target cell with a certain gene of interest, or—in the case of an RNA construct being transfected- to translate the corresponding protein of interest from a transfected mRNA. For vectors operating on the level of transcription and subsequent translation, the gene of interest is under control of a promoter sequence and the promoter sequence is operational inside the target cell, thus, the gene of interest is transcribed either constitutively or in response to a stimulus or dependent on the cell's status. In certain embodiments, the viral genome is packaged into a capsid to become a viral vector, which is able to transduce the target cell.

### Binding; Binders Ligands Antibodies:

The term specific binding in the context of the present invention refers to a property of ligands that bind to their target with a certain affinity and target specificity. The affinity of such a ligand is indicated by the dissociation constant of the ligand. A specifically reactive ligand has a dissociation constant of ≤10⁻⁷ mol/L (particularly ≤10⁻⁹ mol/L) when binding to its target, but a dissociation constant at least three orders of magnitude higher in its interaction with a molecule having a globally similar chemical composition as the target, but a different three-dimensional structure.

In the context of the present specification, the term dissociation constant (K_{D}) is used in its meaning known in the art of chemistry and physics; it refers to an equilibrium constant that measures the propensity of a complex composed of [in most cases, two] different components to dissociate reversibly into its constituent components. The complex can be e.g. an antibody-antigen complex AbAg composed of antibody Ab and antigen Ag. K_{D} is expressed in molar concentration [mol/l] and corresponds to the concentration of [Ab] at which half of the binding sites of [Ag] are occupied, in other words, the concentration of unbound [Ab] equals the concentration of the [AbAg] complex. The dissociation constant can be calculated according to the following formula:

$${K}_{D} = \frac{[ Ab ] * [ Ag ]}{[ AbAg ]}$$

[Ab]: concentration of antibody; [Ag]: concentration of antigen; [AbAg]: concentration of antibody-antigen complex

In the context of the present specification, the terms off-rate (K_{off}; [1/sec]) and on-rate (Kₒₙ; [L/(sec*mol)]) are used in their meaning known in the art of chemistry and physics; they refer to a rate constant that measures the dissociation (K_{off}) or association (Kₒₙ) of an antibody with its target antigen. K_{off} and Kₒₙ can be experimentally determined using methods well established in the art. A method for determining the K_{off} and Kₒₙ of an antibody employs surface plasmon resonance. This is the principle behind biosensor systems such as the Biacore® or the ProteOn® system. They can also be used to determine the dissociation constant K_{D} by using the following formula:

$${K}_{D} = \frac{[ {K}_{off} ]}{[ {K}_{on} ]}$$

The natural upper limit for the on-rate Kₒₙ is 10⁹ L/(sec*mol).

In the context of the present specification, the term antibody refers to whole antibodies including but not limited to immunoglobulin type G (IgG), type A (IgA), type D (IgD), type E (IgE) or type M (IgM), any antigen-binding fragment or single chains thereof and related or derived constructs. A whole antibody is a glycoprotein comprising at least two heavy (H) chains and two light (L) chains inter-connected by disulfide bonds. Each heavy chain is comprised of a heavy chain variable region (VH) and a heavy chain constant region (C_{H}). The heavy chain constant region of IgG is comprised of three domains, C_{H}1, C_{H}2 and C_{H}3. Each light chain is comprised of a light chain variable region (abbreviated herein as VL) and a light chain constant region (CL). The light chain constant region is comprised of one domain, CL. The variable regions of the heavy and light chains contain a binding domain that interacts with an antigen. The binding specificity of the antibody is largely determined by the complementarily determining regions (CDR). Numbering systems identifying amino acid residues in the CDR regions as used in this specification is laid out in FIG. 1. The constant regions of the antibodies may mediate the binding of the immunoglobulin to host tissues or factors, including various cells of the immune system (e.g., effector cells) and the first component of the classical complement system. Similarly, the term encompasses a so-called nanobody or single domain antibody, an antibody fragment consisting of a single monomeric variable antibody domain.

In the context of the present specification, the term fragment crystallizable (Fc) region is used in its meaning known in the art of cell biology and immunology; it refers to a fraction of an antibody comprising, if applied to IgG, two identical heavy chain fragments consisting of a C_{H}2 and a C_{H}3 domain, covalently linked by disulfide bonds.

In the context of the specification, the term single-chain variable fragment (scFv) refers to a fusion protein in which the variable regions of the light chain, and heavy chain variable domains of the Fab fragment of a monoclonal antibody are joined by means of a flexible peptide linker. Multimeric scFv can be created by joining two or more pairs of heavy and light chain domains by peptide linkers.

In the context of the present specification, the term humanized antibody refers to an antibody originally produced by immune cells of a non-human species, the protein sequences of which have been modified to increase their similarity to antibody variants produced naturally in humans. The term humanized antibody as used herein includes antibodies in which CDR sequences derived from the germline of another mammalian species, such as a mouse, have been grafted onto human framework sequences. Additional framework region modifications may be made within the human framework sequences as well as within the CDR sequences derived from the germline of another mammalian species.

### (Cancer) Immunotherapy

In the context of the present specification, the term anti-PD-1 antagonist antibody is meant to encompass an agent, particularly an antibody (or antibody-like molecule) capable of disrupting the signal cascade leading to T cell inhibition after T cell activation as part of what is known in the art the immune checkpoint mechanism. Non-limiting examples include antibodies to PD-1 (Uniprot Q15116) such as clinically available antibody drugs nivolumab (Bristol-Myers Squibb; CAS No 946414-94-4), pembrolizumab (Merck Inc.; CAS No. 1374853-91-4), dostarlimab (Tesaro; CAS No 2022215-59-2), sintilimab (Eli Lilly, InnoVent Biologics; CAS No 2072873-06-2), tislelizumab (BeiGene; CAS No 1858168-59-8), cemiplimab (CAS No 1801342-60-8), cetrelimab (CAS No 2050478-92-5), sasanlimab (Cas No 2206792-50-7), toripalimab (CAS No, 1924598-82-2), zeluvalimab (CAS No 2315361-37-4) or ezabenlimab (CAS No 2249882-54-8).

As used herein, the term pharmaceutical composition refers to a compound of the invention, or a pharmaceutically acceptable salt thereof, together with at least one pharmaceutically acceptable carrier. In certain embodiments, the pharmaceutical composition according to the invention is provided in a form suitable for topical, parenteral or injectable administration.

As used herein, the term pharmaceutically acceptable carrier includes any solvents, dispersion media, coatings, surfactants, antioxidants, preservatives (for example, antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington: the Science and Practice of Pharmacy, ISBN 0857110624).

The term cancer as used in the context of the present specification relates to malignant neoplastic disease; the terms “cancer” and “malignant neoplastic disease” are used synonymously herein. They specifically include carcinoma (epithelial derived cancer), sarcoma (connective tissue derived cancer), lymphoma and leukemia, germ-cell derived tumors and blastomas. Particular alternatives of any of the aspects and embodiments disclosed herein are directed at the use of the compounds and compositions of the invention in treatment of solid tumors. Other alternatives of any of the aspects and embodiments disclosed herein are directed at the use of the combinations of the invention in treatment of liquid cancers such as myelogenous or granulocytic leukemia, particularly AML, lymphatic, lymphocytic, or lymphoblastic leukemia and lymphoma, polycythemia vera or erythremia.

As used herein, the term treating or treatment of any disease or disorder (e.g. cancer) refers in one embodiment, to ameliorating the disease or disorder (e.g. slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. Methods for assessing treatment and/or prevention of disease are generally known in the art, unless specifically described hereinbelow.

### DETAILED DESCRIPTION OF THE INVENTION

A first aspect of the invention is an immunoconjugate comprising an anti-PD-1 antibody binding domain, an anti-IL-2 antibody antigen binding domain, and an IL-2 polypeptide. The IL-2 polypeptide is covalently linked to amino acid residues of the anti-IL-2 binding domain, optionally by means of one, or two peptide linkers, to provide a single contiguous recombinant polypeptide, resulting in an immunoconjugate biased IL-2 signaling to the dimeric IL-2 receptor. The features of each of these functional components are elaborated upon in the sections below.

### Anti-PD1 Binding Domains

The anti-PD1 binding domain of the immunoconjugate according to the invention is the antigen-binding domain portion of an anti-PD-1 antibody. In other words, it comprises a heavy chain polypeptide and a light chain polypeptide derived from an antibody capable of binding specifically to the extracellular domain of the human PD-1 protein. PD-1 specificity is conferred by an antibody heavy chain variable domain polypeptide (PD1-VH), and an antibody light chain variable domain polypeptide (PD1-VL) comprising the antibody CDR and surrounding framework residues.

The anti-PD-1 binding domain, and thus the immunoconjugate as a whole, does not block the interaction between PD-1 and current, commercially/clinically used antagonistic CPi antibodies targeting PD-1, including pembrolizumab, and nivolumab. The PD-1 antagonist non-blocking feature of the immunoconjugate, allows the compound to be administered as a therapeutic agent targeted to PD-1+ cells, in combination with existing, anti-PD-1 checkpoint blockade antibodies that are regulatorily approved. The immunoconjugate targets PD-1+ cells to deliver IL-2 signaling to exhausted PD-1+ cells, without disturbing the effects of a co-administered CPi compound (Table 17, FIG. 3). Example 13 further demonstrates that the immunoconjugate does not inhibit the natural ligand for PD-1, PD-L1, from binding to PD-1.

Significant inhibition of a PD-1 antagonist antibody by immunoconjugate binding to said cell can be determined by measuring the % of immunoconjugate binding to a sample of human PD-1+ cells (e.g. Jurkat PD-1+ cells), following preincubation of the sample with 100× molar excess of pembrolizumab, or nivolumab. The mean fluorescence intensity (MFI) of the immunoconjugate in a pembrolizumab, or nivolumab pre-blocked sample, is no more than 20%, particularly 15%, lower than a control, untreated sample.

In certain embodiments, binding of the immunoconjugate to a PD-1+ cell is not significantly inhibited by PD-1 antagonistic antibodies, particularly by pembrolizumab or nivolumab.

In certain embodiments, binding of the immunoconjugate to recombinant PD-1 does not inhibit binding of recombinant PD-L1 to PD-1.

The inventors used multiple methods to arrive at suitable anti-PD-1 binding domains according to the invention. Firstly, by optimizing the affinity, clinical safety features (e.g. cross-reactivity), and pharmacokinetic properties (e.g. stability) of non-blocking antibodies to develop antibodies with more favorable clinical properties. A second approach developed suitable novel monoclonal antibodies, by screening hybridomas for non-blocking of pembrolizumab, or nivolumab, as well as multiple parameters such as PD-1 affinity, tissue cross reactivity, heavy and light chain liabilities, and immunogenicity.

In some embodiments of the immunoconjugate according to the invention, the anti-PD-1 binding domain is derived from one of the antibodies disclosed herein as the optimized clones derived from the parent clone z2-XVT458, assigned as z2-XVT458m1-m6 (SEQ ID NOS 057 to 068). In alternative embodiments, the anti-PD-1 binding domain is derived from a clone selected from those disclosed in Tables 14, 15, or 16. Such antibodies show favorable high-affinity binding to PD-1, and cross reactivity to human and primate PD-1 to enable clinical testing in primate models.

In some embodiments of the immunoconjugate according to the invention, the PD1-VH comprises an HCDR1 having the sequence GFTFSINAMT (SEQ ID NO 118), an HCDR2 having the sequence TISGSGFSTYYADSLKGR (SEQ ID NO 119), and an HCDR3 having the sequence EVYGDY (SEQ ID NO 120). In addition, the PD1-VL comprises an LCDR1 having the sequence SGX¹SSNIGSX²X³VF (SEQ ID NO 121); wherein X¹ is N, S, Q, or A, and where X²X³ is NS, QS, SS, or NA), an LCDR2 having the sequence SNNQRPS (SEQ ID NO 122), and an LCDR3 having the sequence AAWDDSLSIWVF (SEQ ID NO 123). In particular embodiments, X¹ is selected from the list consisting of S, Q, or A; and X²X³ is a pair of amino acid residues selected from QS, SS, or NA.

This encompasses PD-1 binding domains tested in the examples with high affinity for PD-1 with modified deamination and glycosylation sites compared to the LCDR1 of the high-affinity anti-PD-1 parent clone 21A08 (see SEQ ID NO 071, 072).

In some embodiments of the immunoconjugate according to the invention, the PD1-VH comprises an HCDR1 having the sequence GFTFSINAMT (SEQ ID NO 118), an HCDR2 having the sequence TISGSGFSTYYADSLKGR (SEQ ID NO 119), and an HCDR3 having the sequence EVYGDY (SEQ ID NO 120). In addition, the PD1-VL comprises an LCDR1 having the sequence SGASSNIGSQSVF (SEQ ID NO 124), an LCDR2 having the sequence SNNQRPS (SEQ ID NO 122), and an LCDR3 having the sequence AAWDDSLSIWVF (SEQ ID NO 123). Such embodiments relate to an anti-PD-1 binding domain having the CDR of clone 21A08Ap1, a high affinity anti-PD-1, non-blocking antibody, with little cross-reactivity to other proteins, binding to both human and primate PD-1. In particular embodiments, the full heavy and light chain variable domain are identical to that of 21A8Ap1, wherein the PD1-VH comprises a polypeptide having the sequence SEQ ID NO 085 and the PD1-VL comprises a polypeptide having the sequence SEQ ID NO 086. In more particular embodiments, the PD1-VH consists of SEQ ID NO 085, and the PD1-VL consists of SEQ ID NO 086.

In some embodiments of the immunoconjugate according to the invention, the PD1-VH comprises an HCDR1 having the sequence GFTFSINAMT (SEQ ID NO 118), an HCDR2 having the sequence TISGSGFSTYYADSLKGR (SEQ ID NO 119), and an HCDR3 having the sequence EVYGDY (SEQ ID NO 120). In addition, the PD1-VL comprises an LCDR1 having the sequence SGASSNIGSSSVF (SEQ ID NO 125), an LCDR2 having the sequence SNNQRPS (SEQ ID NO 122), and an LCDR3 having the sequence AAWDDSLSIWVF (SEQ ID NO123). Such embodiments relate to an anti-PD-1 binding domain having the CDR of clone 21A08Ap2, a high affinity anti-PD-1, non-blocking antibody, with little cross-reactivity to other proteins, binding to both human and primate PD-1. In particular embodiments, the full heavy and light chain variable domain are identical to that of 21A8Ap2, wherein the PD1-VH comprises a polypeptide having the sequence SEQ ID NO 085 and the PD1-VL comprises a polypeptide having the sequence SEQ ID NO 087. In more particular embodiments, the PD1-VH consists of SEQ ID NO 085, and the PD1-VI consists of SEQ ID NO 087.

In some embodiments of the immunoconjugate according to the invention, the PD1-VH comprises an HCDR1 having the sequence GFTFSINAMT (SEQ ID NO 118), an HCDR2 having the sequence TISGSGFSTYYADSLKGR (SEQ ID NO 119), and an HCDR3 having the sequence EVYGDY (SEQ ID NO 120). In addition, the PD1-VL comprises an LCDR1 having the sequence SGASSNIGSNAVF (SEQ ID NO 126), an LCDR2 having the sequence SNNQRPS (SEQ ID NO 122), and an LCDR3 having the sequence AAWDDSLSIWVF (SEQ ID NO 123). Such embodiments relate to an anti-PD-1 binding domain having the CDR of clone 21A08Ap3, a high affinity anti-PD-1, non-blocking antibody binding to both human and primate PD-1. In particular embodiments, the full heavy and light chain variable domain are identical to that of 21A8Ap3, wherein the PD1-VH comprises a polypeptide having the sequence SEQ ID NO 085 and the PD1-VL comprises a polypeptide having the sequence SEQ ID NO 091. In more particular embodiments, the PD1-VH consists of SEQ ID NO 085, and the PD1-VI consists of SEQ ID NO 091.

In some embodiments of the immunoconjugate according to the invention, the PD1-VH comprises an HCDR1 having the sequence GFTFSISAMT (SEQ ID NO 135), an HCDR2 having the sequence TISGSGGSTYYSDSVKG (SEQ ID NO 136), and an HCDR3 having the sequence EVYGDY (SEQ ID NO 120). In addition, the PD1-VL comprises an LCDR1 having the sequence SGSSSN/GSNS (SEQ ID NO 126), an LCDR2 having the sequence SNNQRPS (SEQ ID NO 122), and an LCDR3 having the sequence AAWDDSLSIWVF (SEQ ID NO 123). Such embodiments relate to an anti-PD-1 binding domain having the CDR of clone 22F13, a high affinity anti-PD-1, non-blocking antibody binding to both human and primate PD-1.

In some embodiments of the immunoconjugate according to the invention, the PD1-VH comprises an HCDR1 having the sequence GFTFSTYAMS (SEQ ID NO 138), an HCDR2 having the sequence TISGTGYSTYFADSVKG (SEQ ID NO 139), and an HCDR3 having the sequence EVYGDY (SEQ ID NO 120). In addition, the PD1-VL comprises an LCDR1 having the sequence SGRSSNIGSNS (SEQ ID NO 140), an LCDR2 having the sequence SNNQRPS (SEQ ID NO 122), and an LCDR3 having the sequence AAWDDSLSGWV (SEQ ID NO 141). Such embodiments relate to an anti-PD-1 binding domain having the CDR of clone 25120, a high affinity anti-PD-1, non-blocking antibody binding to both human and primate PD-1.

In some embodiments of the immunoconjugate according to the invention, the PD1-VH comprises an HCDR1 having the sequence GFTFSSYS (SEQ ID NO 142), an HCDR2 having the sequence FISSSSPTLYYADSVKG (SEQ ID NO 143), and an HCDR3 having the sequence ARQGLTPFDY (SEQ ID NO 144). In addition, the PD1-VL comprises an LCDR1 having the sequence RASQSVSSYLA (SEQ ID NO 145), an LCDR2 having the sequence GASTRAT (SEQ ID NO 146), and an LCDR3 having the sequence QQYNNWPYT (SEQ ID NO 147). Such embodiments relate to an anti-PD-1 binding domain having the CDR of clone 20H02, a high affinity anti-PD-1, non-blocking antibody binding to both human and primate PD-1.

In some embodiments of the immunoconjugate according to the invention, the PD1-VH comprises an HCDR1 having the sequence GFTFSNYGMH (SEQ ID NO 148), an HCDR2 having the sequence VIWYDGSKKYYADSVKG (SEQ ID NO 149), and an HCDR3 having the sequence NSGH (SEQ ID NO 150). In addition, the PD1-VL comprises an LCDR1 having the sequence RSSQSLLDSDDGNTYLD (SEQ ID NO 151), an LCDR2 having the sequence TLSYRAS (SEQ ID NO 152), and an LCDR3 having the sequence MQCIEFPHT (SEQ ID NO 153). Such embodiments relate to an anti-PD-1 binding domain having the CDR of clone 39F23, a high affinity anti-PD-1, non-blocking antibody binding to both human and primate PD-1.

In some embodiments of the immunoconjugate according to the invention, the PD1-VH comprises an HCDR1 having the sequence GFTFSSYSMN (SEQ ID NO 154), an HCDR2 having the sequence YITSSSNTMYYADSVKG (SEQ ID NO 155), and an HCDR3 having the sequence IVGAPFDY(SEQ ID NO 156). In addition, the PD1-VL comprises an LCDR1 having the sequence RASQSVSSSLA (SEQ ID NO 157), an LCDR2 having the sequence GASTRAT (SEQ ID NO 146), and an LCDR3 having the sequence QQYNNWPFT (SEQ ID NO 158). Such embodiments relate to an anti-PD-1 binding domain having the CDR of clone 40B20, a high affinity anti-PD-1, non-blocking antibody binding to both human and primate PD-1.

In some embodiments of the immunoconjugate according to the invention, the PD1-VH comprises an HCDR1 having the sequence GYTFTSYFMH (SEQ ID NO 159), an HCDR2 having the sequence LINPDGGNTDYAQKFQG (SEQ ID NO 160), and an HCDR3 having the sequence DGNYYDSSGYYYPDAFDI (SEQ ID NO 161). In addition, the PD1-VL comprises an LCDR1 having the sequence QGDSLRRFYAS (SEQ ID NO 162), an LCDR2 having the sequence GKDNRPS (SEQ ID NO 163), and an LCDR3 having the sequence NSRDSSGTHVV (SEQ ID NO 164). Such embodiments relate to an anti-PD-1 binding domain having the CDR of clone 56H02, a high affinity anti-PD-1, non-blocking antibody binding to both human and primate PD-1.

In further embodiments of the immunoconjugate according to the invention, the anti-PD-1 binding domain is derived from the parent clone z2-XVT458, having high affinity for both human and primate PD-1. In addition to the CDR mutations, the sequences of z2-XVT458-m1, z2-XVT458-m3 and z2-XVT458-m6 contain a G57D mutation immediately downstream of the CDRL2, which may contribute to the increased affinity of these clones.

In certain embodiments of the immunoconjugate according to the invention, the PD1-VH comprises an HCDR1 having the sequence NFYIH (SEQ ID NO 127), an HCDR2 having the sequence RIYPNYGITAYNQKFKD (SEQ ID NO 128), and an HCDR3 having the sequence GYSYAMDY (SEQ ID NO 129). In addition, the PD1-VL comprises an LCDR1 having the sequence SASQGISGDLN (SEQ ID NO 130), an LCDR2 having the sequence HTSQRHS (SEQ ID NO 131), and an LCDR3 having the sequence QGYSKDLLT (SEQ ID NO 132). Such embodiments encompass an anti-PD-1 binding domain having CDR of clone XVT458-Z2-M3. This clone has high affinity binding to human and primate PD-1, as well as little cross-reactivity to other human protein antigens. In particular embodiments, the full heavy and light chain variable domain are identical to that of XVT458-Z2-M3, wherein the PD1-VH comprises, or consists of a polypeptide having the sequence SEQ ID NO 061 and the PD1-VL comprises, or consists of a polypeptide having the sequence SEQ ID NO 062.

In certain embodiments of the immunoconjugate according to the invention, the PD1-VH comprises an HCDR1 having the sequence NFYIH (SEQ ID NO 127), an HCDR2 having the sequence SIYPNYGITAYNQKFKD (SEQ ID NO 133), and an HCDR3 having the sequence GYSYAMDY (SEQ ID NO 129). In addition, the PD1-VL comprises an LCDR1 having the sequence SASQGISGDLN (SEQ ID NO 130), an LCDR2 having the sequence HTSQLHS (SEQ ID NO 134), and an LCDR3 having the sequence QGYSKDLLT (SEQ ID NO 132). Such embodiments encompass an anti-PD-1 binding domain having CDR of clone XVT458-Z2-m6. This clone has high affinity binding to human and primate PD-1, as well as little cross-reactivity to other human protein antigens. In particular embodiments, the full heavy and light chain variable domain are identical to that of XVT458-Z2-m6, wherein the PD1-VH comprises, or consists of a polypeptide having the sequence SEQ ID NO 067 and the PD1-VL comprises, or consists of a polypeptide having the sequence SEQ ID NO 068.

In certain embodiments of the immunoconjugate according to the invention, the affinity constant (K_{D}) of the immunoconjugate for PD-1 is in the range of 4.0×10⁻⁹ to 1×10⁻¹¹ mol/L, wherein binding affinity is determined using surface plasmon resonance in accordance with the protocol given in Example 2. In particular embodiments the K_{D} is 2.0×10⁻⁹ to 1.5×10⁻¹⁰ mol/L. In particular embodiments, the anti-PD-1 domain confers very high affinity to PD-1, where the K_{D} is within the range of 2×10⁻⁹ to 2×10⁻¹¹ mol/L.

In certain other embodiments of the immunoconjugate according to the invention, the affinity constant (K_{D}) of the immunoconjugate for PD-1 is in the range of 4.0×10⁻⁸ to 1.5×10⁻¹⁰ mol/L, wherein binding affinity is determined using surface plasmon resonance in accordance with the protocol given in Example 2. In particular embodiments the K_{D} is 2.0×10⁻⁸ to 1.5×10⁻¹ mol/L.

In certain embodiments, the anti-PD1 antigen binding domain is a variant of 21A08. In particular embodiments thereof, the PD1-VH comprises, or consists of a polypeptide at least (≥) 95%, 98%, 99% similar to SEQ ID NO 085 (VH 21A08), and the PD1-VL comprises, or consists of a polypeptide ≥95%, 98%, 99% similar to SEQ ID NO 086 (VL 21A08Ap1). In particular embodiments, the PD1-VH comprises, or consists of a polypeptide a polypeptide ≥95%, 98%, 99% similar to SEQ ID NO 085 (VH 21A08) and the PD1-VL comprises, or consists of a polypeptide ≥95%, 98%, 99% similar to SEQ ID NO 087 (VL 21A08Ap2). In particular embodiments, the PD1-VH comprises, or consists of a polypeptide a polypeptide ≥95%, 98%, 99% similar to SEQ ID NO 085 (VH 21A08) and the PD1-VL comprises, or consists of a polypeptide ≥95%, 98%, 99% similar to SEQ ID NO 091 (VL 21A08Ap3). These variants of 21A08 are characterized by a K_{D} to PD-1 of 1.0×10⁻⁹ to 1.5×10⁻¹¹.

In certain embodiments, the anti-PD1 antigen binding domain is a variant of XVT458. In particular embodiments thereof, the PD1-VH comprises, or consists of a polypeptide ≥95%, 98%, 99% similar to SEQ ID NO 061 (VH XVT458-Z2-M3) and the PD1-VL comprises, or consists of a polypeptide ≥95%, 98%, 99% similar to SEQ ID NO 062 (VL XVT458-Z2-M3). In some embodiments, the PD1-VH comprises, or consists of a polypeptide ≥95%, 98%, 99% similar to SEQ ID NO 067 (VH XVT458-Z2-M6) and the PD1-VL comprises, or consists of a polypeptide ≥95%, 98%, 99% similar to SEQ ID NO 068 (VL XVT458-Z2-M6). These variants of XVT458 are characterized by a K_{D} to PD-1 of 2.0×10⁻⁹ to 1.0×10⁻¹⁰.

### IL-2 Polypeptides and the Anti-IL-2 Binding Domain

The anti-IL-2 binding domain comprises a heavy chain polypeptide and a light chain polypeptide derived from an anti-IL-2 antibody capable of binding specifically to the human IL-2 protein. An IL-2 polypeptide is covalently linked to amino acid residues of the anti-IL-2 binding domain, optionally by means on one, or two peptide linkers, to provide a single contiguous recombinant polypeptide, providing biased signaling to the dimeric IL-2 receptor.

The anti-IL-2 binding domain comprises firstly, an antibody heavy chain variable domain polypeptide (IL2-VH) having from N′ to C′ terminus the following domains: a first framework region (IL2VH₁), a first complementarity determining region (IL2HCDR1), a second framework region (IL2VH₂), a second complementarity determining region (IL2HCDR2), a third framework region (IL2VH₃), a third complementarity determining region (IL2HCDR3), and a fourth framework region (IL2VH₄). The anti-IL-2 binding domain further comprises an antibody light chain variable domain polypeptide (IL2-VL) having from N′ to C′ terminus the following domains: a first framework region (IL2VL₁), a first complementarity determining region (IL2LCDR1), a second framework region (IL2VL₂), a second complementarity determining region (IL2LCDR2), a third framework region (IL2VL₃), a third complementarity determining region (an IL2LCDR3), and a fourth framework region (IL2VL₄).

Embodiments of an IL-2 polypeptide portion of the immunoconjugate according to the invention include human IL-2, artificial IL-2 variant polypeptides such as Proleukin, or IL-2 muteins as provided in Table 2. The IL-2 polypeptide according to the invention lacks the signal peptide of M1 to S21 of native IL-2 SEQ ID NO 005. An IL-2 polypeptide comprises the following functional regions, a first alpha helix (A), a second alpha helix (B), a third alpha helix (C) and a fourth alpha helix (D) as indicated in Table 2. In some embodiments, the IL-2 polypeptide is a mutein, a recombinant IL-2 protein comprising one or more amino acid substitutions or deletions to modify function, such as reduce binding to CD25. In particular embodiments, the immunoconjugate comprises a mutein selected from those listed in Table 2. A mutein, or IL-2 variant polypeptide according to the invention is >90%, particularly 95%, 98% similar to SEQ ID NO 005 and has equivalent biological function to SEQ ID NO 005. Non-limiting examples of variants and muteins are disclosed as SEQ ID NO 006 and 007. Biological function for an IL-2 polypeptide is defined according to the invention as an affinity for the dimeric IL-2 receptor in the range of the K_{D} of the immunoconjugate for the dimeric IL-2 receptor is in the range of 500 nM to 0.1 nM, particularly 100 nM to 0.1 nM, more particularly 10 nM to 0.1 nM, still more particularly wherein the K_{D} is in the range of 1 nM to 0.1 nM, as measured by surface plasmon resonance (SPR).

### Circularly Permuted IL-2 Polypeptides

In particular embodiments of the immunoconjugate according to the invention, the IL-2 polypeptide is a circularly permuted IL-2 (IL2CP), having functional domains reordered relative to their domain position in the wildtype sequence of SEQ ID NO 005, such as the examples provided in in Table 3. Particular embodiments of an IL-2 polypeptide starting sequence to which circular permutation can be applied are WT SEQ ID NO 005, SEQ ID NO 006 or the muteins listed in the Table 2.

A circularly permuted IL-2 polypeptide is created by “opening” an IL-2 polypeptide sequence, such as SEQ ID NO 005 or 006, to create a new N′ terminus and C′ terminus, as well as joining natural N′ and C′ terminal amino acid residues (see SEQ ID NO 010 to 013 for examples of the resulting circularly permuted interleukin sequence). This provides a reordered IL2CP polypeptide retaining the tertiary structure of human IL-2 (see for example WO2013184942A1). In some embodiments, the IL-2 polypeptide is opened between two consecutive amino acids. In other embodiments, the IL-2 polypeptide is opened between two positions within one region (thus potentially excising 1, or several amino acids of the IL-2 polypeptide as in the structures disclosed in WO2013184942A1). In certain embodiments, the new IL2CP N- and C-termini are amino acids no more than 2 amino acids apart in the base IL-2 polypeptide sequence. In more particular embodiments, the IL2CP C- and N-termini are adjacent residues in the human IL-2 polypeptide or mutein, such that the IL2CP comprises all amino acid residues of the base IL-2 polypeptide sequence, with none excised.

In particular embodiments of the immunoconjugate, the anti-IL-2 binding domain is joined to an IL2CP in which an N-terminal amino acid of the human IL-2 polypeptide or variant thereof, particularly an N-terminal amino acid selected from A21, P22, T23, S24, S25, or S26, is joined to a C-terminal amino of the human IL-2 polypeptide or variant thereof selected from T150, L152, and T153. In more particular embodiments, the IL-2 residue P22, is joined to T153.

Importantly, the IL2CP it is opened at a location that does not disrupt an alpha helix structure. The inventors identified such regions between the C and B helix, the A and B helix, and between the C and D helix. In particular embodiments, both the new N-terminus, and the C-terminus located within the region V89 to D104, particularly S95 to N97, more particularly K96 and N97. This provides an IL2CP cut between the C/B, where the alpha helices of the IL2CP are in the order CDAB. In some embodiments, both the new N-terminus, and the C-terminus located within the region G47 to E72, particularly L56 to position E72, more particularly K63 to F64. This provides an IL2CP cut between the A/B, where the alpha helices of the IL2CP are in the order BCDA. In some embodiments, both the new N-terminus, and the C-terminus located within the region G118 to 1134, particularly between S119 to A132. This provides an IL2CP cut between the C/D, where the alpha helices of the IL2CP are in the order DABC.

### Fusion of Anti-IL-2 Binding Domain and IL-2 Polypeptides

In the immunoconjugate according to the invention, one of the antibody variable domains within the heavy chain or the light chain of the anti-IL-2 antigen binding domain is joined, optionally by means of one or two peptide linkers, to an IL-2 polypeptide. Together, the anti-IL-2 binding domain joined to the IL-2 polypeptide, delivers biased IL-2 signaling to the dimer IL-2 receptor expressed on exhausted CD8 T cells. In some embodiments, the IL2CP N-terminus or the L2CP C-terminus is covalently linked, optionally by means of one or two peptide linkers to the IL2-VL, or the IL2-VH. In some embodiments, the IL2CP N-terminus or the L2CP C-terminus is covalently linked, optionally by means of one, or two peptide linkers to the IL2-VL, and the IL2-VH, in other words, the IL2CP is embedded within the IL2-VL or IL2-VH.

In some embodiments of the immunoconjugate according to invention, both the IL2CP N′ terminus and IL2 CP C′ terminus are covalently linked, optionally by means of one, or two peptide linkers, to amino acid residues within a domain selected from the list consisting of IL2HCDR1, IL2HCDR2, IL2VH₃, IL2HCDR3, IL2LCDR1, IL2LCDR2, IL2VL₃ or IL2LCDR3. Crystal structure analysis by the inventors suggests these are particularly helpful sites for embedding an IL2CP to ensure optimal placement of the cytokine with reference to an anti-IL-2 binding domain, preserving biased IL-2 signaling signals to IL-2 receptors.

In some embodiments of the immunoconjugate according to invention, the anti-IL-2 binding domain is fused to an IL2CP at least (≥) 98% similar to SEQ ID NO 010, the IL2CP CDAB variant 1 validated in Example 1. In some embodiments of the immunoconjugate according to invention, the anti-IL-2 binding domain is fused to an IL2CP at least (≥) 98% similar to SEQ ID NO 011, the IL2CP CDAB variant 2 validated in Example 1. In these embodiments, the IL2CP N-terminus and IL2CP C-terminus are covalently linked, directly, or by means of one, or two peptide linkers, to amino acid residues within a domain selected from the list consisting of IL2LCDR1, IL2LCDR2, IL2HCDR1, or IL2HCDR3, particularly IL2LCDR1, or IL2HCDR3. In particular embodiments, a CDAB IL2CP polypeptide is embedded within IL2LCDR1.

In some embodiments of the immunoconjugate according to invention, the anti-IL-2 binding domain is joined to an IL2CP at least (≥) 98% similar to SEQ ID NO 012, the IL2CP variant 3 BCDA. In these embodiments, the IL2CP N-terminus and IL2CP C-terminus are covalently linked, optionally by means of one or two peptide linkers, to amino acid residues within a domain selected from the list consisting of IL2LCDR1, IL2LCDR3, IL2HCDR1, IL2HCDR2, IL2VH₃. In a particular embodiment, the IL2CP N-terminus and IL2CP C-terminus are linked to IL2HCDR2. An IL2CP in the BCDA format may also be joined to the anti-IL-2 binding domain at the end of IL2VL₄ by means of a flexible linker.

In some embodiments of the immunoconjugate according to invention, the anti-IL-2 binding domain is joined to an IL2CP at least (≥) 98% similar to SEQ ID NO 013, the IL2CP variant 3 DABC. In these embodiments, the IL2CP N-terminus and IL2CP C-terminus are preferably covalently linked, optionally by means of one, or two peptide linkers, to amino acid residues within a domain selected from the list consisting of IL2HCDR2 and IL2VH₃.

In particular embodiments of the immunoconjugate according to the invention, no amino acids “0”, or 1, 2, 3, or 4, particularly 2, consecutive amino acids within a domain selected from the list consisting of IL2HCDR1, IL2HCDR2, IL2VH₃, IL2HCDR3, IL2LCDR1, IL2LCDR2, IL2VL₃, or IL2LCDR3, have been replaced with a polypeptide consisting of, listed from N- to C-direction: a first peptide linker, an IL2CP as described in the preceding five paragraphs, and a second peptide linker. In reference to this aspect of the invention “0” means the sequences is inserted into the IL2-VH or IL2-VL region, but no amino acids from the anti-IL-2 binding domain antibody portion are lost; “1” means the IL2CP is inserted into the place where the one lost amino acid was, “2” is the same but two amino acids from the IL-2-VH or VL were lost, or replaced by the IL2CP, and so on.

In particular embodiments of the immunoconjugate according the invention, it comprises an IL2CP embedded within the IL-2-VH, or the IL-2-VL, flanked, or joined, at each end by a short peptide linker. In more particular embodiments, both the first and second peptide linkers are 1-20 amino acids in length. In still more particular embodiments both the first and second peptide linkers are between 2 and 7 amino acids in length. In further particular embodiments of the immunoconjugate according to the invention, any peptide linker flanking the IL2CP is comprised of glycine (G), or G and serine (S) residues. In more particular embodiments, the peptide linker joining the C- or N-terminal residue of the IL2 polypeptide to amino acids of the IL-2VH or IL-VL has a sequence selected from the sequences assigned as SEQ ID NO 014-024.

### N-Terminal Linked IL-2 or IL2CP

In alternative embodiments of the immunoconjugate according to the invention, the IL-2 polypeptide (optionally an IL2CP) is covalently linked by means of a peptide linker to an N-terminal amino acid residue of the IL2VL₄ domain, or to an N-terminal amino acid residue of the IL2VH₄ domain. In particular embodiments, the N-terminal peptide linker is between 10 to 30, more particularly 21 or 22 amino acids in length. Cytokines can be linked to antibody binding domains when combined with medium linkage peptides, allowing flexibility of positioning of the cytokine with the antibody binding domain, however longer peptide linkers can allow trans interactions between molecules which can lead to formation of aggregates.

### Receptor-Biased IL-2 Signaling

In particular embodiments of the immunoconjugate according to the invention, the immunoconjugate is characterized in that the K_{D} of the immunoconjugate for the dimeric IL-2 receptor (comprising CD122 and CD132) is in the range of 500 nM to 0.1 nM, 100 nM to 0.1 nM, 10 nM to 0.1 nM, particularly wherein the K_{D} is in the range of 1 nM to 0.1 nM. Binding affinity according to this aspect of the invention is to be determined using surface plasmon resonance. Binding of Fab fragment of representative compound comprising an IL-2-anti-IL-2-binding domain fusion according to the invention (QTY065) to CD122:CD132 is 0.322 nM, as measured by SPR in the examples at 200 nM. A further aspect of receptor biased binding can be characterized as the immunoconjugate having no detectable binding to CD25, as measured by SPR at 200 nM.

### Anti-IL-2 Binding Domains

The designation of CDR provided in embodiments of the anti-IL-2 binding domain provided herein are based on an uninterrupted antibody sequence, and is understood that the sequence may be interrupted as defined herein, by an IL2CP fused within the IL2-VH, or the IL2-VL. Otherwise, it is clear that alignment to the canonical CDR sequences would not be recognized. Therefore, the term CDR according to the invention encompasses not just canonical CDR amino acids, but may be a longer recombinant polypeptide in which those residues aligning to the canonical CDR residues are interrupted by an IL2CP, and optionally, peptide linkers joining the antibody binding region of the immunoconjugate to the IL-2 polypeptide domain.

In certain embodiments of the immunoconjugate according to the invention, the IL2-VH is >95%, particularly more than >98%, or 99% identical to SEQ ID NO 043 (VH Antibody A), and the IL2-VL is >95% particularly more than >98%, or 99% identical to SEQ ID NO 042 (VL Antibody A), and has a similar biological function to Antibody A (and with the proviso that the IL-2 polypeptide, or peptide linkers optionally embedded in one of said variable chains according to e.g. claim 2 are excluded from the amino acid alignment/sequence identity). According to these embodiments, the affinity constant (K_{D}) of an antibody characterized by the IL-2VH and the IL2-VL for an IL-2 polypeptide having SEQ ID NO 006 (Proleukin) is in the range of 500 nM to 2 nM, particularly 270 nM to 2 nM, more particularly about 2 nM. The determination of this affinity constant (K_{D}) refers to the interaction of the corresponding free antibody as employed as an anti-IL-2 binding domain according to the invention, and free IL-2 (not the immunoconjugate wherein those two features are linked).

In certain embodiments of the immunoconjugate according to the invention, the anti-IL-2 antigen binding domain comprises, or consists of, an IL2-VH having sequence SEQ ID NO 043 (Antibody A VH), associated with an IL2-VL selected from SEQ ID NO 025 to 031. Such embodiments encompass the LCDR1 IL2CP-embedded Antibody A structures EAD409, XFO227, QTY065, FJC828, PG035, DRV470, XUB802. In particular embodiments the anti-IL-2 binding domain comprises, or consists of SEQ ID NO 043 and SEQ ID NO 027.

In certain embodiments of the immunoconjugate according to the invention, the anti-IL-2 antigen binding domain comprises, or consists of, an IL2-VL having sequence SEQ ID NO 042 (Antibody A VL), associated with an IL2-VH selected from SEQ ID NO 046, or 047. Such embodiments relate to the Antibody A-based CDRH3 (VBE401) or CDRH2 (L1707) IL2CP-embedded structures, each comprising alternative validated IL2CP sequences.

In further embodiments of the immunoconjugate according to the invention, the IL2-VH is >95%, particularly more than >98%, or 99% identical to SEQ ID NO 044 (VH Antibody B), and the IL2-VL is >95 particularly more than >98%, or 99% identical to SEQ ID NO 050 (VL Antibody B). In such embodiments, the affinity constant (K_{D}) of an antibody characterized by the IL-2VH and the IL2-VL for an IL-2 polypeptide having SEQ ID NO 006 (Proleukin) is in the range of 500 nM to 2 nM, particularly 270 nM to 2 nM.

In certain embodiments of the immunoconjugate according to the invention, the anti-IL-2 antigen binding domain comprises, or consists of, an IL2-VH having sequence SEQ ID NO 044 (Antibody B VH), associated with an IL2-VL selected from SEQ ID NO 032 to 036. These embodiments relate to IL2CP embedded into LCDR1 of Antibody B, EPK959, GYG794, DRO069, or ECV200, and BFC885. These also showed little binding to CD25 as measured by SPR (Table 5). These validated alternatives of IL2CP composition and linkage embedding, deliver biased signaling to the dimeric IL-2 receptor according to the invention.

### Fc Portions

In certain embodiments of the immunoconjugate according to the invention, the immunoconjugate comprises an Fc portion, conferring extended half-life in vivo. In particular embodiments, the Ig Fc is an IgG Fc portion.

In certain embodiments of the immunoconjugate, the IgG Fc portion is characterized by the presence of one or more modifications to constant regions of the heavy chains to enhance correct heavy chain pairing. In particular embodiments, the modifications are selected from the following knob and hole paired mutations to enhance heavy chain pairing:
Knob: S354C, T366W and Hole: Y349C, T366S, L368A, Y407V;
Knob: T366Y, and Hole Y407T;
Knob: Y349C T366W, and Hole: S354C, T366S, L368A, Y407V;
Knob: T366W, and Hole: Y407A, T366S, L368A.

In certain embodiments of the immunoconjugate according to the invention, the IgG Fc portion is characterized by the presence of one or more modifications to constant regions of the heavy chains to reduce effector function of the Fc portion. In particular embodiments, said modification, or modifications are selected from L234A, L235A (LALA), L234A, L235A, P329G (LALA-PG), L234A, L235A, P329A (LALA-PA), N297A, N297Q, N297G and D265A, N297G (DANG). In particular embodiments, the immunoconjugate comprises P329A (LALA-PA).

### Immunoconjugate Formats

In particular embodiments of the immunoconjugate according to the invention, the immunoconjugate is a heterotetrameric IgG consisting of a first heterodimer comprising the anti-PD-1 binding domain, and a second heterodimer comprising the anti-IL2 binding domain. This format is illustrated in FIG. 4, and includes such formats as a heterotetrameric kappa/lambda IgG format, and the Crossmab format (FIG. 4C, WO2009 080253).

In particular embodiments of the immunoconjugate according the invention, the immunoconjugate is a heterotetrameric kappa/lambda IgG format, having an anti-PD-1 binding domain is characterized by a lambda light chain, for example derived from the 21A08 antibody clones listed herein (FIG. 4F).

In certain embodiments of the immunoconjugate according to the invention, the immunoconjugate comprises, or consists of the polypeptides having the sequences SEQ ID NO 111, SEQ ID NO 095, SEQ ID NO 112, and SEQ ID NO 052 (NZA596).

In certain embodiments of the immunoconjugate according to the invention, the immunoconjugate comprises, or consists of the polypeptides having the sequences SEQ ID NO 094, SEQ ID NO 095, SEQ ID NO 100, and SEQ ID NO 052 (XWY176).

In certain embodiments of the immunoconjugate according to the invention, the immunoconjugate comprises, or consists of the polypeptides having the sequences SEQ ID NO 097, SEQ ID NO 098, SEQ ID NO 100, and SEQ ID NO 052 (GQM289).

In certain embodiments of the immunoconjugate according to the invention, the immunoconjugate comprises, or consists of the polypeptides having the sequences SEQ ID NO 117, SEQ ID NO 104, SEQ ID NO 112, and SEQ ID NO 052 (BGY642).

In alternative embodiments of the immunoconjugate according to the invention, the immunoconjugate is an immunoglobulin ScFv format as illustrated in FIGS. 4D and 4E. This format comprises an anti-PD1 antibody comprising a first and a second anti-PD-1 binding domain. Both the first and the second binding domain are characterized by an antibody heavy chain comprising a PD1-VH and an antibody light chain comprising a PD1-VL as specified in the section Anti-PD1 binding domains. The format further comprises an anti-IL-2 ScFv comprising an anti-IL-2 binding domain as specified in the section anti-IL-2 binding domains. In the immunoglobulin ScFv format, the C-terminal residue of the anti-IL-2 ScFv IL2-VH, or the IL2-VL domain is linked via a peptide linker to the N-terminal residues of the heavy chain, or the light chain of the anti-PD1 antibody. In particularly embodiments, said peptide linker is 10 to 30 amino acids in length. In still more particular embodiments, said peptide linker has the sequence (G4S)2 or SEQ ID NO 024.

In certain embodiments of the immunoconjugate according to the invention, the immunoconjugate comprises, or consists of the polypeptides having the sequences SEQ ID NO 094, 095, and 096. In certain embodiments of the immunoconjugate according to the invention, the immunoconjugate comprises, or consists of the polypeptides having the sequences SEQ ID NO 097, 098, and 099. In certain embodiments of the immunoconjugate according to the invention, the immunoconjugate comprises, or consists of the polypeptides having the sequences SEQ ID NO 105, 106, and 107. In certain embodiments of the immunoconjugate according to the invention, the immunoconjugate comprises, or consists of the polypeptides having the sequences SEQ ID NO 108, 109, and 110. In certain embodiments of the immunoconjugate according to the invention, the immunoconjugate comprises, or consists of the polypeptides having the sequences SEQ ID NO 113, 106, and 114. In certain embodiments of the immunoconjugate according to the invention, the immunoconjugate comprises, or consists of the polypeptides having the sequences SEQ ID NO 115, 109, and 116. These can all be represented by the format of FIG. 4E.

### Further Aspects of the Invention

Another aspect of the invention relates to an isolated nucleic acid encoding the immunoconjugate according to any one of the embodiments of the immunoconjugate aspect of the invention. Another aspect of the invention relates to an expression vector comprising said isolated nucleic acid. The invention further encompasses a host cell comprising said nucleic acid or said expression vector.

Another aspect of the invention relates to a combination medicament comprising
i) an immunoconjugate as specified in any one of the aspects or embodiments of the invention herein, and
ii) an anti-PD-1 antagonist antibody.

In particular embodiments the anti-PD1 antagonist antibody is selected from the list consisting of nivolumab, pembrolizumab, dostarlimab, sintilimab, tislelizumab, cemiplimab, cetrelimab, sasanlimab, toripalimab, zeluvalimab, or ezabenlimab. In more particular embodiments, the antibody is cemiplimab, dostarlimab, zeluvalimab, tiselizumab, ezabenlimab, toripalimab, cetrelimab, nivolumab or pembrolizumab. In still more particular embodiments, the antibody is nivolumab or pembrolizumab.

### Medical Treatment and Medical Use

Similarly, within the scope of the present invention is a method of treating cancer in a patient in need thereof, comprising administering to the patient an immunoconjugate according to the above aspects and embodiments of the description.

In some embodiments, the immunoconjugate is an heterotetrameric immunoglobulin characterized by two heavy chains and two light chains, one pair of which forms an IL-2 binding ligand and is fused to an IL-2 polypeptide (as in FIG. 4C or E). In some embodiments, the immunoconjugate consists of an anti-PD-1 antibody, an associated pair of heavy and light chain heterodimers, joined by means of a peptide linker to a scFv comprising anti-IL-2 antibody heavy and light chain variable domains, one of which anti-IL-2 domain is joined (optionally by means of peptide linkers) to an IL-2 polypeptide (as in FIG. 4 D or E).

Another aspect of the invention, is an immunoconjugate comprising a non-blocking anti-PD-1 binding domain according to the invention, for use in a patient who is receiving an anti-PD1 antagonist antibody. In particular embodiments, the anti-PD-1 antibody is selected from the list consisting of nivolumab, pembrolizumab, dostarlimab, sintilimab, tislelizumab, cemiplimab, cetrelimab, sasanlimab, toripalimab, zeluvalimab and ezabenlimab. In more particular embodiments, the antibody is cemiplimab, dostarlimab, zeluvalimab, tiselizumab, ezabenlimab, toripalimab, cetrelimab, nivolumab or pembrolizumab.

In some embodiments the immunoconjugate according to the invention is administered to a patient who has received, or is scheduled to receive, treatment with an anti-PD-1 antagonist antibody within 6 weeks, particularly within 3 weeks, of immunoconjugate.

Another aspect of the invention is an anti-PD1 antagonist antibody for use in a patient receiving administration of an immunoconjugate according to the invention. In particular embodiments, the anti-PD-1 antibody is selected from the list consisting of nivolumab, pembrolizumab, dostarlimab, sintilimab, tislelizumab, cemiplimab, cetrelimab, sasanlimab, toripalimab, zeluvalimab and ezabenlimab In certain embodiments for use in a patient who has been administered the immunoconjugate according to the invention with the previous month. In some embodiments, the patient has received treatment with an immunoconjugate according to any one of the embodiments of the invention within the last 6 weeks, or is scheduled to receive treatment with the immunoconjugate in the next 6 weeks,

The invention further encompasses nivolumab for use in a patient who is receiving administration of an immunoconjugate according to the invention. The invention further encompasses pembrolizumab for use in a patient who is receiving administration of an immunoconjugate according to the invention.

Similarly, the invention encompasses methods of treatment of a patient having been diagnosed with a cancer. This method entails administering to the patient an effective amount of immunoconjugate as identified herein, its pharmaceutically acceptable salt, as specified in detail herein, optionally in combination with an anti-PD1 antagonist antibody. In particular embodiments, the same subject is co-administered, within a medically relevant window, with an anti-PD1 antagonist antibody.

Pharmaceutical Compositions. Administration/Dosage Forms and Salts

According to one aspect of the compound according to the invention, the immunoconjugate according to the invention is provided as a pharmaceutical composition, pharmaceutical administration form, or pharmaceutical dosage form, said pharmaceutical composition, pharmaceutical administration form, or pharmaceutical dosage form comprising the immunoconjugate of the present invention and at least one pharmaceutically acceptable carrier, diluent or excipient.

In certain embodiments of the invention, the immunoconjugate of the present invention is typically formulated into pharmaceutical dosage forms to provide an easily controllable dosage of the drug and to give the patient an elegant and easily handled product.

The invention further encompasses a pharmaceutical composition comprising an immunoconjugate of the present invention, or a pharmaceutically acceptable salt thereof, and a pharmaceutically acceptable carrier. In further embodiments, the composition comprises at least two pharmaceutically acceptable carriers, such as those described herein.

Certain embodiments of the invention relate to a dosage form for parenteral administration, such as subcutaneous, intravenous, intrahepatic or intramuscular injection forms. Optionally, a pharmaceutically acceptable carrier and/or excipient may be present.

The pharmaceutical compositions of the present invention can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc. They may be produced by standard processes, for instance by conventional mixing, granulating, dissolving or lyophilizing processes. Many such procedures and methods for preparing pharmaceutical compositions are known in the art, see for example L. Lachman et al. The Theory and Practice of Industrial Pharmacy, 4th Ed, 2013 (ISBN 8123922892).

### Method of Manufacture and Method of Treatment According to the Invention

The invention further encompasses, as an additional aspect, the use of an immunoconjugate as specified in detail above, for use in a method of manufacture of a medicament for the treatment or prevention of a cancer.

Similarly, the invention encompasses methods of treatment of a patient having been diagnosed with a cancer. This method entails administering to the patient an effective amount of immunoconjugate as identified herein, its pharmaceutically acceptable salt, as specified in detail herein, optionally in combination with an anti-PD-1 antagonist antibody.

Wherever alternatives for single separable features such as, for example, an isotype protein or coding sequence, or cancer are laid out herein as “embodiments”, it is to be understood that such alternatives may be combined freely to form discrete embodiments of the invention disclosed herein. Thus, any of the alternative embodiments for an immunoconjugate may be combined with any of the alternative embodiments of an anti-PD-1 antagonist compound and these combinations may be combined with any medical indication mentioned herein.

The invention further encompasses the following items:
A. An immunoconjugate comprising, or consisting of the polypeptides having the sequences SEQ ID NO 111, SEQ ID NO 095, SEQ ID NO 112, and SEQ ID NO 052 (NZA596).
B. An immunoconjugate comprising, or consisting of the polypeptides having the sequences SEQ ID NO 094, SEQ ID NO 095, SEQ ID NO 100, and SEQ ID NO 052 (XWY176).
C. An immunoconjugate comprising, or consisting of the polypeptides having the sequences SEQ ID NO 097, SEQ ID NO 098, SEQ ID NO 100, and SEQ ID NO 052 (GQM289).
D. An immunoconjugate comprising, or consisting of the polypeptides having the sequences SEQ ID NO 117, SEQ ID NO 104, SEQ ID NO 112, and SEQ ID NO 052 (BGY642).
E. An immunoconjugate comprising, or consisting of the polypeptides having the sequences SEQ ID NO 094, 095, and 096 (LTJ498).
F. An immunoconjugate comprising, or consisting of the polypeptides having the sequences SEQ ID NO 097, 098, and 099 (JLI141).
G. An immunoconjugate comprising, or consisting of the polypeptides having the sequences SEQ ID NO 105, 106, and 107 (TMU471).
H. An immunoconjugate comprising, or consisting of the polypeptides having the sequences SEQ ID NO 108, 109, and 110 (MDS446). 1. An immunoconjugate comprising, or consisting of the polypeptides having the sequences s SEQ ID NO 113, 106, and 114 (KTX917).
J. An immunoconjugate comprising, or consisting of the polypeptides having the sequences SEQ ID NO 115, 109, and 116 (CIT348).
K. An immunoconjugate comprising, or consisting of the polypeptides having the sequences SEQ ID NO 103, 104, 090 and 052 (QAB373)
L. An immunoconjugate comprising, or consisting of the polypeptides having the sequences SEQ ID NO 101, 102, 090 and 052 (RIB426)

The following set of items is further encompassed by the invention:
1. An immunoconjugate comprising
   i. an anti-PD1 antigen binding domain, comprising
   an antibody heavy chain variable domain polypeptide (PD1-VH), and
   an antibody light chain variable domain polypeptide (PD1-VL);
   ii. an anti-IL-2 antigen binding domain, comprising
   an antibody heavy chain variable domain polypeptide (IL2-VH) having from N′ to C′ terminus the following domains: a first framework region (IL2VH₁), a first complementarity determining region (IL2HCDR1), a second framework region (IL2VH₂), a second complementarity determining region (IL2HCDR2), a third framework region (IL2VH₃), a third complementarity determining region (IL2HCDR3), and a fourth framework region (IL2VH₄); and
   an antibody light chain variable domain polypeptide (IL2-VL) having from N′ to C′ terminus the following domains: a first framework region (IL2VL₁), a first complementarity determining region (IL2LCDR1), a second framework region (IL2VL₂), a second complementarity determining region (IL2LCDR2), a third framework region (IL2VL₃), a third complementarity determining region (an IL2LCDR3), and a fourth framework region (IL2VL₄); and
   iii. an IL-2 polypeptide,
      wherein the IL-2 polypeptide is covalently linked to the IL2-VL, or the IL2-VH, to provide a single contiguous recombinant polypeptide;
      characterized in that
      binding of pembrolizumab, or nivolumab to a PD-1+ cell does not significantly inhibit binding of the immunoconjugate to said cell.
2. The immunoconjugate according to item 1, wherein
   the PD1-VH comprises an HCDR1 having the sequence GFTFSINAMT (SEQ ID NO 118), an HCDR2 having the sequence TISGSGFSTYYADSLKGR (SEQ ID NO 119), and an HCDR3 having the sequence EVYGDY (SEQ ID NO 120); and
   the PD1-VL comprises an LCDR1 having the sequence SGX¹SSNIGSX²X³VF (SEQ ID NO 121), an LCDR2 having the sequence SNNQRPS (SEQ ID NO 122), and an LCDR3 having the sequence AAWDDSLSIWVF (SEQ ID NO 123);
   wherein X¹ is N, S, Q, or A, particularly wherein X¹ is S, Q, or A;
   and wherein X²X³ is NS, QS, SS, or NA, particularly wherein X²X³ is QS, SS, or NA.
3. The immunoconjugate according to item 1 or 2, wherein the PD1-VL comprises an LCDR1 having the sequence SGASSNIGSQSVF (SEQ ID NO 124).
4. The immunoconjugate according to item 3, wherein the PD1-VH comprises or consists of a polypeptide having the sequence SEQ ID NO 085 and the PD1-VL comprises or consists of a polypeptide having the sequence SEQ ID NO 086.
5. The immunoconjugate according to item 1 or 2, wherein the PD1-VL comprises an LCDR1 having the sequence SGASSNIGSSSVF (SEQ ID NO 125).
6. The immunoconjugate according to item 5, wherein the PD1-VH comprises or consists of a polypeptide having the sequence SEQ ID NO 085 and the PD1-VL comprises or consists of a polypeptide having the sequence SEQ ID NO 087.
7. The immunoconjugate according to item 1 or 2, wherein the PD1-VL comprises an LCDR1 having the sequence SGASSNIGSNAVF (SEQ ID NO 126)
8. The immunoconjugate according to item 7, wherein the PD1-VH comprises or consists of a polypeptide having the sequence SEQ ID NO 085 VH 21A08 and the PD1-VL comprises or consists of a polypeptide having the sequence SEQ ID NO 91.
9. The immunoconjugate according to any one of the items 1 to 8, wherein
   the PD1-VH comprises, or consists of a polypeptide at least (≥) 95%, 98%, 99% similar to SEQ ID NO 085, and the PD1-VL comprises, or consists of a polypeptide ≥95%, 98%, 99% similar to SEQ ID NO 086;
   the PD1-VH comprises, or consists of a polypeptide a polypeptide ≥95%, 98%, 99% similar to SEQ ID NO 085 and the PD1-VL comprises, or consists of a polypeptide ≥95%, 98%, 99% similar to SEQ ID NO 087;
   the PD1-VH comprises, or consists of a polypeptide a polypeptide ≥95%, 98%, 99% similar to SEQ ID NO 085; and the PD1-VL comprises, or consists of a polypeptide ≥95%, 98%, 99% similar to SEQ ID NO 091;
   and wherein the affinity constant (K_{D}) of the immunoconjugate for PD-1 is in the range of 1.0×10⁻⁹ to 1.5×10⁻¹⁰ mol/L as measured according to the protocol provided in Example 2.
10. The immunoconjugate according to item 1, wherein:
   the PD1-VH comprises an HCDR1 having the sequence NFYIH (SEQ ID NO 127), an HCDR2 having the sequence RIYPNYGITAYNQKFKD (SEQ ID NO 128), and an HCDR3 having the sequence GYSYAMDY (SEQ ID NO 129); and
   the PD1-VL comprises an LCDR1 having the sequence SASQGISGDLN (SEQ ID NO 130), an LCDR2 having the sequence HTSQRHS (SEQ ID NO 131), and an LCDR3 having the sequence QGYSKDLLT (SEQ ID NO 132).
11. The immunoconjugate according to item 10, wherein the PD1-VH comprises, or consists of a polypeptide having the sequence SEQ ID NO 061 and the PD1-VL comprises, or consists of a polypeptide having the sequence SEQ ID NO 062.
12. The immunoconjugate according to item 1, wherein:
   the PD1-VH comprises an HCDR1 having the sequence NFYIH (SEQ ID NO 127), an HCDR2 having the sequence SIYPNYGITAYNQKFKD (SEQ ID NO 133), and an HCDR3 having the sequence GYSYAMDY (SEQ ID NO 129); and
   the PD1-VL comprises an LCDR1 having the sequence SASQGISGDLN (SEQ ID NO 130), an LCDR2 having the sequence HTSQLHS (SEQ ID NO 134), and an LCDR3 having the sequence QGYSKDLLT (SEQ ID NO 132);
13. The immunoconjugate according to item 12, wherein the PD1-VH comprises, or consists of a polypeptide having the sequence SEQ ID NO 067 and the PD1-VL comprises, or consists of a polypeptide having the sequence SEQ ID NO 068.
14. The immunoconjugate according to any one of the items 1, 10 to 13, wherein
   the PD1-VH comprises, or consists of a polypeptide ≥95%, 98%, 99% similar to SEQ ID NO 061 and the PD1-VL comprises, or consists of a polypeptide ≥95%, 98%, 99% similar to SEQ ID NO 062; or
   wherein the PD1-VH comprises, or consists of a polypeptide ≥95%, 98%, 99% similar to SEQ ID NO 067 and the PD1-VL comprises, or consists of a polypeptide ≥95%, 98%, 99% similar to SEQ ID NO 068;
   and wherein the affinity constant (Kd) of the immunoconjugate for PD-1 is in the range of 2×10⁻⁹ to 2×10−1 mol/L as measured according to the protocol provided in Example 2.
15. The immunoconjugate according to any one of the items 1 to 14, wherein the IL-2 polypeptide is a circularly permuted IL-2 (IL2CP) polypeptide;
   wherein in comparison to a human IL-2 polypeptide or variant thereof, said IL2CP is characterized in that:
   an N-terminal amino acid of the human IL-2 polypeptide or variant thereof is joined to a C-terminal amino of the human IL-2 polypeptide or variant thereof, and
   said IL-2 polypeptide or variant thereof is opened at a location that does not disrupt an alpha helix structure, to provide an IL2CP N-terminus and an IL2CP C-terminus; and
   wherein the IL2CP N-terminus and/or the L2CP C-terminus is covalently linked to the IL2-VL, or the IL2-VH.
16. The immunoconjugate according to item 15, wherein an N-terminal amino acid selected from A21, P22, T23, S24, S25, or S26 of the human IL-2 polypeptide or variant thereof is joined to a C-terminal amino acid of the human IL-2 polypeptide or variant thereof selected from T150, L152, and T153.
17. The immunoconjugate according to item 16, wherein the N-terminal amino acid is P22 and the C-terminal amino acid is T153.
18. The immunoconjugate according to any one of items 15 to 17, wherein both the IL2CP N-terminus and the IL2CP C-terminus are located within V89 to D104, particularly S95 to N97, more particularly K96 and N97.
19. The immunoconjugate according to any one of items 15 to 17, wherein both the IL2CP N-terminus and the IL2CP C-terminus are located within G47 to E72, particularly L56 to position E72, more particularly K63 to F64.
20. The immunoconjugate according to any one of items 15 to 17, wherein both the IL2CP N-terminus and the IL2CP C-terminus are located within G118 to 1134, particularly S119 to A132.
21. The immunoconjugate according to any one of the items 15 to 20, wherein both the IL2CP N′-terminus and IL2 CP C′-terminus are covalently linked, optionally by means of one, or two peptide linkers, to amino acid residues within a domain selected from the list consisting of IL2HCDR1, IL2HCDR2, IL2VH₃, IL2HCDR3, IL2LCDR1, IL2LCDR2, IL2VL₃ or IL2LCDR3.
22. The immunoconjugate according to any one of the items 15 to 20, wherein the IL2CP is ≥98% similar to SEQ ID NO 010 or SEQ ID NO 011; and
   the IL2CP N′-terminus and IL2CP C′-terminus are covalently linked, optionally by means of one, or two peptide linkers, to amino acid residues within a domain selected from the list consisting of IL2LCDR1, IL2LCDR2, IL2HCDR1, or IL2HCDR3, particularly IL2LCDR1, or IL2HCDR3.
23. The immunoconjugate according to any one of the items 15 to 20, wherein the IL2CP is ≥98% similar to SEQ ID NO 012; and
   the IL2CP N′-terminus and IL2CP C′-terminus are covalently linked, optionally by means of one, or two peptide linkers, to amino acid residues within a domain selected from the list consisting of IL2LCDR1, IL2LCDR3, IL2HCDR1, IL2HCDR2, IL2VH₃, IL2VL₄, particularly IL2HCDR2.
24. The immunoconjugate according to any one of the items 15 to 20, wherein the IL2CP is ≥98% similar to SEQ ID NO 013; and
   the IL2CP N′-terminus and IL2CP C′-terminus are covalently linked, optionally by means of one, or two peptide linkers, to amino acid residues within a domain selected from the list consisting of IL2HCDR2, or IL2VH₃.
25. The immunoconjugate according to any one of the items 15 to 24, wherein 0, 1, 2, 3, or 4, particularly 2, consecutive amino acids within a domain selected from the list consisting of IL2HCDR1, IL2HCDR2, IL2VH₃, IL2HCDR3, IL2LCDR1, IL2LCDR2, IL2VL₃, or IL2LCDR3, have been replaced with a polypeptide consisting of:
   a first peptide linker,
   the IL2CP,
   a second peptide linker;
   wherein both the first and second peptide linkers are 1-20 amino acids in length, particularly wherein both the first and second peptide linkers are between 2 and 7 amino acids in length.
26. The immunoconjugate according to any one of the items 1 to 14, wherein the IL-2 polypeptide is covalently linked by means of a peptide linker to an N-terminal amino acid residue of the IL2VL₄ domain, or to an N′-terminal amino acid residue of the IL2VH₄ domain, particularly a peptide linker 10 to 30, more particularly 21 or 22 amino acids in length.
27. The immunoconjugate according to any one of the items 1 to 26, wherein the immunoconjugate is characterized in that
   the affinity constant (K_{D}) of the immunoconjugate for the dimeric IL-2 receptor is in the range of 500 nM to 0.1 nM, particularly 100 nM to 0.1 nM, more particularly 10 nm to 0.1 nM, still more particularly wherein the K_{D} is in the range of 1 nM to 0.1 nM; and
   it has no detectable binding to CD25.
28. The immunoconjugate according to any one of the items 1 to 27, wherein the IL2-VH is ≥95%, particularly more than >98%, or 99% identical to SEQ ID NO 043, and the IL2-VL is ≥95% particularly more than >98%, or 99% identical to SEQ ID NO 042; and
   wherein the K_{D} of an antibody characterized by the IL-2VH and the IL2-VL for an IL-2 polypeptide having SEQ ID NO 006 is in the range of 500 nM to 2 nM, particularly 270 nM to 2 nM, more particularly about 2 nM.
29. The immunoconjugate according to any one of the items 1 to 27, wherein the anti-IL-2 antigen binding domain comprises, or consists of, an IL2-VH having sequence SEQ ID NO 043, associated with an IL2-VL selected from SEQ ID NO 025 to 031.
30. The immunoconjugate according to any one of the items 1 to 27, wherein the anti-IL-2 antigen binding domain comprises, or consists of, an IL2-VL having sequence SEQ ID NO 042, associated with an IL2-VH selected from SEQ ID NO 046, or 047.
31. The immunoconjugate according to any one of the items 1 to 27, wherein the IL2-VH is ≥95%, particularly more than >98%, or >99% identical to SEQ ID NO 044, and the IL2-VL is ≥95% particularly more than >98%, or >99% identical to SEQ ID NO 050;
   and wherein the K_{D} of an antibody characterized by the IL-2VH and the IL2-VL for an IL-2 polypeptide having SEQ ID NO 006 is in the range of 500 nM to 2 nM, particularly 270 nM to 2 nM.
32. The immunoconjugate according to any one of the items 1 to 27, wherein the anti-IL-2 antigen binding domain comprises, or consists of, an IL2-VH having sequence SEQ ID NO 044, associated with an IL2-VL selected from SEQ ID NO 032 to 036.
33. The immunoconjugate according to any one of items 1 to 32, wherein the immunoconjugate comprises an Fc portion, particularly an IgG Fc portion.
34. The immunoconjugate according to item 33, wherein the IgG Fc portion is characterized by the presence of one or more modifications to constant regions of the heavy chains to enhance correct heavy chain pairing, particularly a set of know and hole modifications selected from:
   knob: S354C, T366W and hole: Y349C, T366S, L368A, Y407V;
   knob: T366Y, and hole Y407T;
   knob: Y349C, T366W, and hole: S354C, T366S, L368A, Y407V; or
   knob: T366W, and Hole: Y407A, T366S, L368A.
35. The immunoconjugate according to any one of the items 33 or 34, wherein the IgG Fc portion is characterized by the presence of one or more modifications to constant regions of the heavy chains to reduce effector function of the Fc portion;
   particularly a modification to constant regions of the heavy chains selected from L234A, L235A (LALA), L234A, L235A, P329G (LALA-PG), L234A, L235A, P329A (LALA-PA), N297A, N297Q, N297G and D265A, N297G (DANG), more particularly P329A (LALA-PA).
36. The immunoconjugate according to any one of the items 1 to 35, wherein the immunoconjugate is a heterotetrameric IgG consisting of
   a first heterodimer comprising the anti-PD-1 binding domain as specified in any in of the items 2 to 14; and
   a second heterodimer comprising the anti-IL2 binding domain as specified in any in of the items 15 to 32.
37. The immunoconjugate according to item 36, wherein the heterotetrameric IgG format is a heterotetrameric kappa/lambda IgG format.
38. The immunoconjugate according to item 36, wherein the heterotetrameric IgG format is a Crossmab format.
39. The immunoconjugate according to item 36 or 37 wherein the heterotetrameric IgG format is a heterotetrameric kappa/lambda IgG format, wherein the anti-PD-1 binding domain is characterized by a lambda light chain.
40. The immunoconjugate according to any one of the items 1 to 39, wherein the immunoconjugate comprises, or consists of the polypeptides having the sequences SEQ ID NO 111, SEQ ID NO 095, SEQ ID NO 112, and SEQ ID NO 052 (NZA596).
41. The immunoconjugate according to any one of the items 1 to 39, wherein the immunoconjugate comprises, or consists of the polypeptides having the sequences SEQ ID NO 117, SEQ ID NO 104, SEQ ID NO 112, and SEQ ID NO 091 (BGY642).
42. The immunoconjugate according to any one of the items 1 to 39, wherein the immunoconjugate comprises, or consists of the polypeptides having the sequences SEQ ID NO 113, 106, and 114 (KTX917).
43. The immunoconjugate according to any one of the items 1 to 39, wherein the immunoconjugate comprises, or consists of the polypeptides having the sequences SEQ ID NO 115, 109, and 116 (CIT348).
44. The immunoconjugate according to any one of the items 1 to 39, wherein the immunoconjugate comprises, or consists of the polypeptides having the sequences SEQ ID NO 094, SEQ ID NO 095, SEQ ID NO 100, and SEQ ID NO 052 (XWY176).
45. The immunoconjugate according to any one of the items 1 to 39, wherein the immunoconjugate comprises, or consists of the polypeptides having the sequences SEQ ID NO 097, SEQ ID NO 098, SEQ ID NO 100, and SEQ ID NO 052 (GQM289).
46. The immunoconjugate according to any one of the items 1 to 35, wherein the immunoconjugate is an immunoglobulin scFv format comprising:
   an anti-PD1 antibody comprising a first and a second anti-PD-1 binding domain, wherein both the first and the second binding domain are characterized by a PD1-VH and an antibody light chain comprising a PD1-VL as specified in any one of the items 2 to 14; and
   an anti-IL-2 scFv comprising the anti-IL-2 binding domain as specified in any one of the items 15 to 21;
   wherein the C-terminal residue of the anti-IL-2 scFv IL2-VH, or the IL2-VL domain is linked via a peptide linker to the N-terminal residues of the heavy chain, or the light chain of the anti-PD1 antibody, particularly wherein said peptide linker is 10 to 30 amino acids in length, still more particularly wherein said peptide linker has the sequence (G4S)2 or SEQ ID NO 024.
47. The immunoconjugate according to item 46, wherein the immunoconjugate comprises, or consists of the polypeptides having the sequences SEQ ID NO 094, 095, and 096.
48. The immunoconjugate according to item 46, wherein the immunoconjugate comprises, or consists of the polypeptides having the sequences SEQ ID NO 097, 098, and 099.
49. The immunoconjugate according to item 46, wherein the immunoconjugate comprises, or consists of the polypeptides having the sequences SEQ ID NO 105, 106, and 107.
50. The immunoconjugate according to item 46, wherein the immunoconjugate comprises, or consists of the polypeptides having the sequences SEQ ID NO 108, 109, and 110.
51. The immunoconjugate according to item 46, wherein the immunoconjugate comprises, or consists of the polypeptides having the sequences SEQ ID NO 113, 106, and 114.
52. The immunoconjugate according to item 46, wherein the immunoconjugate comprises, or consists of the polypeptides having the sequences SEQ ID NO 115, 109, and 116.
53. An isolated nucleic acid encoding the immunoconjugate according to any one of items 1 to 52.
54. An expression vector comprising the isolated nucleic acid according to item 53.
55. A host cell comprising the nucleic acid according to item 53, or an expression vector according to item 54.
56. A combination medicament comprising
   the immunoconjugate as specified in any one of the items 1 to 52, and
   an anti-PD-1 antagonist antibody, particularly wherein the anti-PD1 antagonist antibody is selected from the list consisting of nivolumab, pembrolizumab, dostarlimab, sintilimab, tislelizumab, cemiplimab, cetrelimab, sasanlimab, particularly nivolumab or pembrolizumab.
57. A pharmaceutical composition comprising the immunoconjugate according to anyone of items 1 to 52, the isolated nucleic acid according to item 53, the expression vector according to item 54, the host cell according to item 55, and a pharmaceutically acceptable excipient.
58. An immunoconjugate according to anyone of items 1 to 52, the isolated nucleic acid according to item 53, the expression vector according to item 54, the host cell according to item 55, or the combination of item 56, or the pharmaceutical composition according to item 57, for use as a medicament.
59. An agent selected from the immunoconjugate according to any one of items 1 to 52, the isolated nucleic acid according to item 53, the expression vector according to item 54, the host cell according to item 55, or the combination of item 56, or the pharmaceutical composition according to item 57, for use in treating cancer.
60. A method of treatment comprising the steps of:
i) selecting a suitable patient; and,
ii) administering a therapeutically effective amount of the immunoconjugate according to any one of items 1 to 52, the isolated nucleic acid according to item 53, the expression vector according to item 54, the host cell according to item 55, or the combination of item 56, or the pharmaceutical composition according to item 57.
61. The agent for use according to item 59, wherein the agent is administered within 6 weeks prior to or subsequent to, treatment with an anti-PD-1 antagonist antibody.
62. The agent for use according to item 61, wherein the anti-PD1 antagonist antibody is selected from the list consisting of nivolumab, pembrolizumab, dostarlimab, sintilimab, tislelizumab, cemiplimab, sasanlimab, zeluvalimab, ezabenlimab, toripalimab, or cetrelimab, particularly, wherein the anti-PD-1 antagonist antibody is selected from the list consisting of nivolumab, pembrolizumab, dostarlimab, tislelizumab, cemiplimab, zeluvalimab, ezabenlimab, toripalimab, or cetrelimab.
63. An anti-PD-1 antagonist antibody for use in treating cancer, wherein the anti-PD-1 antagonist antibody is administered within 6 weeks prior to or subsequent to, treatment with an agent as specified in item 59.
64. The anti PD-1 antagonist antibody for use according to item 63, wherein the anti-PD1 antagonist antibody is selected from the list consisting of nivolumab, pembrolizumab, dostarlimab, sintilimab, tislelizumab, cemiplimab, sasanlimab, zeluvalimab, ezabenlimab, toripalimab, or cetrelimab,
   particularly, wherein the anti-PD-1 antagonist antibody is selected from the list consisting of nivolumab, pembrolizumab, dostarlimab, tislelizumab, cemiplimab, zeluvalimab, ezabenlimab, toripalimab, or cetrelimab.

The invention is further illustrated by the following examples and figures, from which further embodiments and advantages can be drawn. These examples are meant to illustrate the invention but not to limit its scope.

### DESCRIPTION OF THE FIGURES

FIG. 1 shows labelling of antibody residues according to accepted formats with standard numbering and Kabat numbering systems.

FIG. 2 shows how IL2-CP formats can be varied to suit embedding in various variable domain sites. (Left) a figure of the IL-2 with the correct orientation for binding to Antibody A or B (Middle) a figure of LCDR1 embedded CDAB IL-2CP in Antibody A, B or C (right) HCDR3 embedded BCDA IL-2CP.

FIG. 3 SPR sensograms (Response Units, RU over Time in seconds) of PD-1 pre-mixed with antibodies or Buffer run as analyte on immobilized PD-L1.

FIG. 4 shows proof of principal formats with various PD-1 binding moiety arrangement, including A. a double scFv-fusion, B. a Fab-double scFC, C. a heterotetrameric IgG (IgG CrossMab), D-E. two bivalent PD-1 IgG antibodies, with two linkage positions of an anti-hIL-2 ScFv F. a heterotetrameric IgG (kappa/lambda).

FIG. 5 shows cell proliferation analyzed by flow cytometry, detecting Ki67+ cells in CD8 T cells, NK cells and Treg cells. Bispecific compounds administered i.v. at 0.2 mg/kg to wt C57BL/6 mice. Blood samples were taken before the injection and on days 3 and 6 after compound administration.

FIG. 6 A) Top, shows representative image of NZA596 compound digested to obtain two Fab fragments and the Fc domain. Bottom shows deconvoluted MS profile of detected Fab fragments, indicating single species with expected Mw.
B) Top, shows representative image of BGY642 compound digested to obtain two Fab fragments and the Fc domain. Bottom shows deconvoluted MS profile of detected Fab fragments, indicating single species with expected Mw.

FIG. 7 Top) Representative image of cis-signaling on non-blocked CTV+ cells and blocked CSFE+ cells. Bottom) % pSTAT5+ cells plotted against NZA596 compound concentration in nM. CFSE+ cells were pre-incubated with 21A08Ap1 antibody, Pembrolizumab or Nivolumab. (n=3)

FIG. 8 shows the number of unique TCR rearrangements in TILs of mice treated with vehicle, NZA594 or QTY065 administered i.v. at 0.2 mg/kg on day 0 and day 3. Genomic DNA was extracted from tumors of mice sacrificed on study day 5 (n=8). Statistical analysis was performed by an unpaired Wilcox Test.

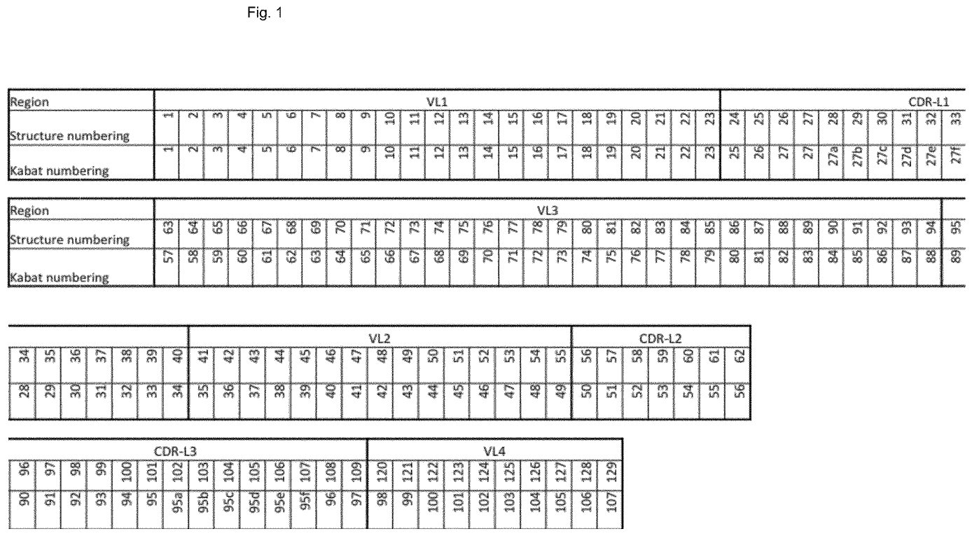

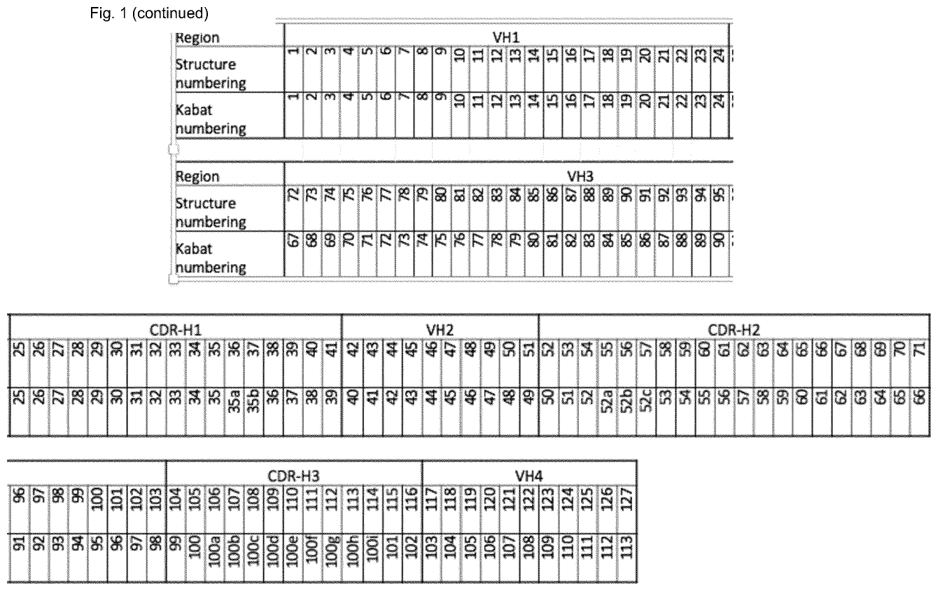

### EXAMPLES

### Example 1: Design and Production of Anti-IL-2 Antibody IL-2 Cytokine Fusion Proteins

Antibodies binding to IL-2 were derived, isolated and structurally characterized according to methods well known to a person skilled in the art. Antibody A (HC SEQ ID NO 001, LC SEQ ID NO 002) is a high-affinity anti-IL-2 antibody and Antibody B (HC SEQ ID NO 003, LC SEQ ID NO 004) is a low-affinity anti-IL-2 antibody. Antibody C has no IL-2 affinity and has been included as a control. The genes encoding for VL-CL (light chain) and VH-CH1-CH2-CH3 (heavy chain) were cloned into the mammalian expression vector pcDNA3.4 into separate plasmids. The antibodies were produced using transient gene expression in Expi293 cells (Gibco, A14527) following standard protocols provided by the vendor. Plasmid DNA (HC/LC ratio 1:2 w/w) was transfected using ExpiFectamine™ 293 Reagent. The cells were maintained at 37° C., 8% CO2 in an orbital shaker (150 rpm) for six days. The antibodies were purified to homogeneity from the supernatant by Protein A Chromatography (MabSelect™ SuRe™, GE17-5438-01). Quality control of the proteins was performed by SDS-PAGE (NuPAGE™ 4-12% Bis-Tris Protein Gels, ThermoFisher) and analytic size exclusion chromatography (SEC) (GE lifesciences, Superdex 200 increase 10/300). Yields and purity are reported in Table 1. All antibodies were obtained as pure protein product, eluting as single peak in SEC-HPLC analysis.

Binding to IL-2 was first tested by ELISA using a serial dilution of antibodies on coated IL-2. Recombinant human IL-2 was coated on Maxisorp ELISA plates (Invitrogen, 44-2404-21) at 5 μg/ml, 4° C. overnight. After 2 hours blocking, antibodies A, B and C were incubated in a serial dilution starting from 10 μg/ml in assay buffer. The antibodies were detected with anti-human IgG-Peroxidase (Sigma, A0170). After addition and blockade of the chemiluminescent substrate, absorption was read (A₄₅₀-A₅₇₀ at plate reader, Spectramax 03). EC₅₀ values were obtained by plotting Absorbance vs. log(Concentration) and sigmoidal 4-PL fitting using GraphPad Prism (Table 1). Only Antibody A displayed binding to IL-2 in ELISA and could be detected on the Fc-portion by an anti-human IgG antibody coupled to HRP. Binding affinities to recombinant human IL-2 (rhIL-2) of Antibodies A, B and C of the present invention were then measured using more sensitive biolayer interferometry (BLI) methodology. Antibodies were immobilized on amine reactive (2^{nd} Generation) sensors (ForteBio, 18-5092). Association (600s) and dissociation (900s) of a dilution series of recombinant IL-2 (Acro Biosystems, IL2-H4113) were measured on the antibody-coated biosensors on an Octet-System (Octet RED, ForteBio). K_{D} values were obtained by fitting the kinetics data with the ForteBio Data Analysis Software (8.2). This assay confirmed Antibody A binds rhIL-2 with high affinity. Binding of Antibody B could not be measured by ELISA, but a weak affinity constant could be determined by BLI. Antibody C did not show binding to rhIL-2 up to concentrations ≥1000 nM (Table 1).

TABLE 1
Production yields from transient expression, purity and binding
affinities to recombinant hIL-2 measured by ELISA or BLI.

| | | Purity | | |
|---|---|---|---|---|
| | Yield | by SEC- | EC₅₀ to IL-2 | K_{D} to IL-2 |
| Antibodies | (mg/ml) | HPLC (%) | by ELISA (nM) | by BLI (nM) |
| A | 193.9 | 95.8% | 0.37 | 2.1 |
| B | 282.4 | 98.9% | — | 269.8 |
| C | 453.5 | 98.5% | — | — |

Fusion proteins comprising an IL-2 polypeptide, or an IL-2 mutein comprising amino acid substitutions with favorable pharmacokinetic properties (Table 2), have previously been demonstrated to provide IL-2 signaling when conjugating the cytokine to an antibody chain by C-terminal linkage to an antibody variable chain region (see for example, WO2018184964A1; Deak L C. et al. 2022, Nature 610:161; Gutbrodt K L. 2013 Sci. Trans. Med. 5:201; Gillies S D. (1992) PNAS 89(4):1428), or N-terminal linkage to an anti-IL-2 antibody (WO2017122130A1).

TABLE 2
IL-2 polypeptides

| SEQ | | |
|---|---|---|
| ID NO | Cytokine or mutein | Sequence |
| SEQ ID NO 5 | Human IL-2 (hIL2, Uniprot P60568) Numbering 1-153 Signal peptide, alpha helix | 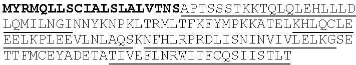 |
| 6 | Proleukin ® (aldesleukin) | MAPTSSSTKKTQLQLEHLLLDLQMILNGINNYKNPKLTRM |
| | C145S | LTFKFYMPKKATELKHLQCLEEELKPLEEVLNLAQSKNFH |
| | | LRPRDLISNINVIVLELKGSETTFMCEYADETATIVEFLN |
| | | RWITFSQSIISTLT |
| 7 | no-alpha mutein | APTSSSTKKTQLQLEHLLLDLQMILNGINNYKNPKLTAML |
| | R58A, F62A, Y65A, E82A | TAKFAMPKKATELKHLQCLEEALKPLEEVLNLAQSKNFHL |
| | | RPRDLISNINVIVLELKGSETTFMCEYADETATIVEFLNR |
| | | WITFCQSIISTLT |
| 8 | W02012/107417A1 | APTSSSTKKTQLQLEHLLLDLQMILNGINNYKNPKLTRML |
| | IL2 mutein | TAKFAMPKKATELKHLQCLEEELKPLEEVLNGAQSKNFHL |
| | F62A, Y65A, L92G | RPRDLISNINVIVLELKGSETTFMCEYADETATIVEFLNR |
| | | WITFCQSIISTLT |
| 9 | IL-2 superkine | APTSSSTKKTQLQLEHLLLDLQMILNGINNYKNPKLTRML |
| | L100F, R101D, L105V, | TFKFYMPKKATELKHLQCLEEELKPLEEVLNLAQSKNFHF |
| | 1106V, 1112F | DPRDVVSNINVFVLELKGSETTFMCEYADETATIVEFLNR |
| | | WITFCQSIISTLT |

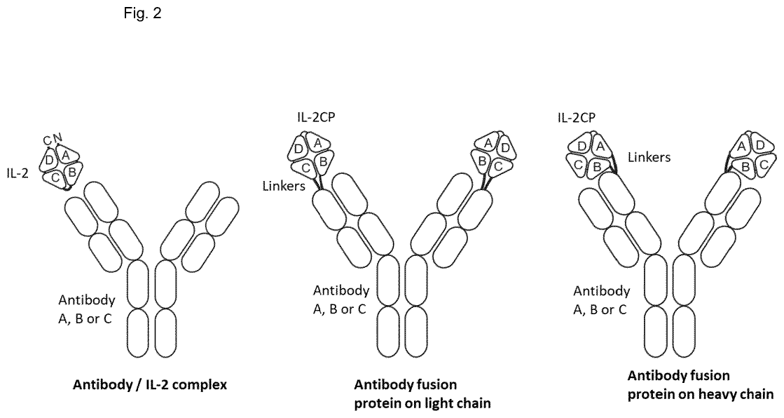

The inventor's assessed an alternative format, analyzing the structure of IL-2 in complex with the antibody NARA (RCSB Protein data bank 5LQB) from which humanized Antibody A and Antibody B were derived, to guide the embedding or fusion of IL-2 to preserve orientation of the cytokine relative to the antibody and similar receptor signaling qualities (Arena-Ramires et al. 2016 Sci. Trans. Med. 8:367). Based on the crystal structure of the NARA1/L-2 complex, possible sites for embedding were identified by checking connecting strands between the different alpha helix domains (assigned here as A-D) and their proximity to CR or R framework regions on the VH or VL of the antibody. Various combinations of circularly permuted IL-2 (IL-2CP) were designed, to retain the cytokine's tertiary structure and orientation with respect to natural binding to the antibody, when fused to the indicated heavy chain or light chain variable regions (Table 3, FIG. 2). Depending on the juncture site, the IL-2 needs to be permuted differently, in order to maintain the same orientation on the antibody. To create a circular permutation with the IL-2 helix domains CDAB format, the crystal structure suggested the region V89 to 0104 (referring to the sequence of Proleukin) between the B and C helix could be opened to maintain key tertiary structures. To create a circular permutation with the IL-2 helix domains BCDA format, the crystal structure suggested the region 047 to E72 between the A and B helix could be opened. To create a circular permutation with the IL-2 helix domains DABC format, the crystal structure suggested the region 0118 to 1134 between the C and D helix could be targeted.

TABLE 3
Circularly permuted IL-2 polypeptides and optimal linkage sites

| SEQ ID | | | |
|---|---|---|---|
| NO | Proleukin variant | Sequence | Fusion |
| 10 | Circularly | NFHLRPRDLISNINVIVLELKGSETTFMCE | CDRL1, CDRL2 |
| | permuted | YADETATIVEFLNRWITFSQSIISTLTPTS | CDRH1, |
| | Proleukin variant 1 | SSTKKTQLQLEHLLLDLQMILNGINNYKNP | CDRH3 |
| | | KLTRMLTFKFYMPKKATELKHLQCLEEELK | |
| | | PLEEVLNLAQSK | |
| 11 | Circularly | FHLRPRDLISNINVIVLELKGSETTFMCEY | CDRL1, CDRL2 |
| | permuted | ADETATIVEFLNRWITFSQSIISTLTPTSS | CDRH1, |
| | Proleukin variant 2 | STKKTQLQLEHLLLDLQMILNGINNYKNPK | CDRH3 |
| | | LTRMLTFKFYMPKKATELKHLQCLEEELKP | |
| | | LEEVLNLAQSK | |
| 12 | Circularly | FYMPKKATELKHLQCLEEELKPLEEVLNLA | CDRL1, CDRL2 |
| | permuted | QSKNFHLRPRDLISNINVIVLELKGSETTF | CDRL3 |
| | Proleukin variant 3 | MCEYADETATIVEFLNRWITFSQSIISTLT | CDRH1, |
| | | PTSSSTKKTQLQLEHLLLDLQMILNGINNY | CDRH2, |
| | | KNPKLTRMLTFK | FW3VH, CDR |
| | | | VL-N terminal |
| 13 | Circularly | ATIVEFLNRWITFCQSIISTLTPTSSSTKK | CDRH2, |
| | permuted | TQLQLEHLLLDLQMILNGINNYKNPKLTRM | FW3VH |
| | Proleukin variant 4 | LTFKFYMPKKATELKHLQCLEEELKPLEEV | |
| | | LNLAQSKNFHLRPRDLISNINVIVLELKGS | |
| | | ETTFMCEYADET | |

Representative IL-2 fusion proteins were then designed according to some embodiments of the present invention comprising Antibodies A, B or C joined to IL-2 either directly, or by means of linkers of various lengths as set out in Table 4.

The LCDR1 of the IL-2 Antibody A Kabat residues Y27d-D30 (or Y31-D34 structure numbering), and the region connecting IL-2 helix B and helix C between residue S95 and N97 were identified as promising regions for further engineering. The LCDR1 of each Antibody A was opened between Y27d and D30 numbered according to Kabat definition, to provide new C-terminal residue at LCDR1 Y27d, and a new N-terminal LCDR1 D30. LCDR1 residue Y27d was joined to the N-terminus of an IL-2CP opened between K96 and N97 (SEQ ID NO 10). The C-terminus of IL-2CP was joined to the LCDR1 residue 30 by the Kabat definition, such that residues Q28 and G29 of LCDR1 of the antibody were replaced by the circularized IL-2CP SEQ ID NO 10, joined directly, or by means of peptide linkers of various lengths. This embedding was repeated for equivalent amino acid residues with LCDR1 of the lower affinity IL-2 Antibody B, and Antibody C having no affinity for IL-2 (Table 4).

Further constructs were designed to validate an alternative IL-2CP insertion in the HDCR3, or HCDR2 region. VBE401 was developed using SEQ ID NO 011, an IL-2 opened between N97 and K96, inserting the cytokine (removing N97 and leaving F96 as new N-terminal end). SEQ ID NO 011 was inserted between residues E98 and G99 in the HCDR3 of Antibody A preceded by the linker GGG, and followed by the linker GGG. In addition, insertion of an IL-2CP into HDRC2 was tested. A circularly permuted IL-2 was created by opening the sequence between K63 and F64, and fusing the original N- and C-termini to provide SEQ ID NO 012. SEQ ID NO 012 was inserted between Antibody A HDCR2 residues G53 and S54, preceded by the linker GG, and followed by the linker GGG as described above (LIZ707).

For each structure (Table 4), the genes encoding VL-(IL-2)-CL, VL-CL, VH-CH1-CH2-CH3, VH-(IL-2)-CH1-CH2-CH3 were cloned into the mammalian expression vector pcDNA3.4 into separate plasmids. The antibody IL-2 fusion proteins were produced using transient gene expression in Expi293 cells as described for antibodies A, B and C above. The purity of all constructs was high (>95%), eluting as single peak in SEC-HPLC analysis (Table 5).

TABLE 4
Antibody-IL-2 fusion proteins, with IL-2CP fusion site on the respective CDR of
antibodies A, B or C. IL-2CP sequence is embedded within the variable domain of
the heavy or light chain of the antibody as indicated. All heavy chains constant
regions were SEQ ID NO 048, all light chain constant regions were SEQ ID NO 049.

| | IL-2 fusion | | | | | |
|---|---|---|---|---|---|---|
| | site, | IL2-CP | Linker 1 | Linker 2 | VL | VH |
| | Antibody | (SEQ ID | (SEQ ID | (SEQ ID | (SEQ ID | (SEQ ID |
| Compound | clone | NO) | NO) | NO) | NO) | NO) |
| EAD406 | CDR-L1, A | 010 | 0 AA | 1 AA | 025 | 043 |
| | | | | (014) | | |
| XFO227 | CDR-L1, A | 010 | 2 AA | 3 AA | 026 | 043 |
| | | | (015) | (016) | | |
| QTY065 | CDR-L1, A | 010 | 3 AA | 4 AA | 027 | 043 |
| | | | (016) | (017) | | |
| FJC828 | CDR-L1, A | 010 | 4 AA | 5 AA | 028 | 043 |
| | | | (017) | (018) | | |
| PGO345 | CDR-L1, A | 010 | 6 AA | 7 AA | 029 | 043 |
| | | | (019) | (020) | | |
| DRV470 | CDR-L1, A | 010 | 13 AA | 14AA | 030 | 043 |
| | | | (021) | (022) | | |
| XUB802 | CDR-L1, A | 010 | 19 AA | 20 AA | 031 | 043 |
| | | | (023) | (024) | | |
| EPK959 | CDR-L1, B | 010 | 0 AA | 1 AA | 032 | 044 |
| | | | | (014) | | |
| GYG794 | CDR-L1, B | 010 | 2 AA | 3 AA | 033 | 044 |
| | | | (015) | (016) | | |
| DRO069 | CDR-L1, B | 010 | 3 AA | 4 AA | 034 | 044 |
| | | | (016) | (017) | | |
| ECV200 | CDR-L1, B | 010 | 6 AA | 7 AA | 035 | 044 |
| | | | (019) | (020) | | |
| BFC885 | CDR-L1, B | 010 | 13 AA | 14AA | 036 | 044 |
| | | | (021) | (022) | | |
| LPT269 | CDR-L1, C | 010 | 0 AA | 1 AA | 037 | 045 |
| | | | | (014) | | |
| DXM339 | CDR-L1, C | 010 | 2 AA | 3 AA | 038 | 045 |
| | | | (015) | (016) | | |
| FUE433 | CDR-L1, C | 010 | 3 AA | 4 AA | 039 | 045 |
| | | | (016) | (017) | | |
| LQM346 | CDR-L1, C | 010 | 6 AA | 7 AA | 040 | 045 |
| | | | (019) | (020) | | |
| GLK754 | CDR-L1, C | 010 | 13 AA | 14AA | 041 | 045 |
| | | | (021) | (022) | | |
| VBE401 | CDR-H3, A | 011 | 3 AA | 3 AA | 042 | 046 |
| | | | (016) | (016) | | |
| LIZ707 | CDR-H2, A | 012 | 2 AA | 3 AA | 042 | 047 |
| | | | (015) | (016) | | |

Immunoconjugate Interactions with the CD132-CD122 Heterodimeric Receptor

The anti-IL-2 antibody clone 5344, binding on the CD122 binding site on IL-2, was used in order to determine correct folding of the IL-2 portion of the fusion proteins. The EC₅₀ values are reported in Table 6. The IL-2 moiety of all the constructs was successfully binding to the anti-IL-2 antibody (clone 5344) in ELISA and could be detected on the Fc-portion by an anti-human IgG antibody coupled to HRP (Table 5).

Binding to the CD122/CD132 complex (His-tagged, Acro Biosystem, Cat #ILG-H5283) of one representative anti-IL-2 antibody/IL-2 fusion (QTY065) was additionally assessed by SPR (BIAcore 3000, v4.1.2; GE Healthcare) and compared to Proleukin. CD122/CD132 was captured on an NTA sensor chip (GE Healthcare). Proleukin or QTY065 Fab were used as analytes. The calculated affinity constant for QTY065 Fab was 0.32 nM, comparable to the value of 0.42 nM obtained with Proleukin. The binding to the intermediate affinity IL-2R complex (CD122/CD132) remains unchanged in the IL-2CP antibody fusion proteins.

TABLE 5
Purity of the final protein by SEC-HPLC and assessment
of functionality by ELISA and CD25 binding by SPR.

| | Purity SEC- | EC₅₀ to 5344 | KD to CD25 |
|---|---|---|---|
| Compound | HPLC (%) | ELISA (nM) | SPR (nM) |
| EAD406 | 97.61 | 0.11 | — |
| XFO227 | 97.12 | 0.14 | — |
| QTY065 | 99.10 | 0.14 | — |
| FJC828 | 97.77 | 0.28 | — |
| PGO345 | 95.75 | 0.16 | — |
| DRV470 | 96.85 | 0.10 | — |
| XUB802 | 94.58 | 0.15 | — |
| EPK959 | 99.20 | 0.15 | — |
| GYG794 | 97.80 | 0.20 | — |
| DRO069 | 97.52 | 0.11 | — |
| ECV200 | 96.80 | 0.13 | — |
| BFC885 | 97.74 | 0.25 | 54.1 |
| LPT269 | 97.47 | 0.24 | 0.12 |
| DXM339 | 97.93 | 0.28 | 0.18 |
| FUE433 | 98.61 | 0.54 | 0.16 |
| LQM346 | 99.09 | 0.28 | 0.15 |
| GLK754 | 98.61 | 0.28 | 0.20 |
| VBE401 | 92.55 | n.d. | — |
| LIZ707 | 91.55 | n.d. | — |

### Biological Action of IL-2 Tested by HEK Blue IL-2 Reporter Assay

The functionality of the IL-2 fusion proteins was assessed using HEK-Blue™ IL-2 reporter cells, engineered to express the trimeric IL-2 receptor (CD25/CD122/CD132) and to secrete secreted embryonic alkaline phosphatase activity (SEAP) upon STAT5 activation triggered by IL-2. HEK-Blue™ IL-2 reporter cells (Invivogen) were incubated with a dilution series (1:3 dilutions from 0.56 nM) of antibody-IL-2 fusion protein in growth medium for 20 hours at 37° C. in 5% CO2. The HEK-Blue™ IL-2 cells supernatant was diluted 1:10 in QUANTI-Blue™ solution, for colorimetric determination of SEAP, and incubated at 37° C. for 3 hours. Absorbances were read at 620 nm and plotting those against protein concentration. E₅₀ values were interpolated using GraphPad Prism. The EC₅₀ values determined by colorimetric determination of SEAP for each concentration of the IL-2 antibody fusion proteins showed antibody IL-2 fusion proteins induced STAT5 signaling on HEK Blue IL-2R reporter cells with comparable potency (Table 6).

TABLE 6
EC50 values of immunoconjugates HEK Blue IL-2 reporter assay

| Compound | EC₅₀ (M) | Compound | EC₅₀ (M) |
|---|---|---|---|
| Proleukin | 4.28E−12 | DRO069 | 3.45E−12 |
| EAD406 | 3.59E−12 | ECV200 | 3.42E−12 |
| XFO227 | 3.03E−12 | BFC885 | 5.09E−12 |
| QTY065 | 3.22E−12 | LPT269 | 7.35E−12 |
| FJC828 | 3.16E−12 | DXM339 | 5.47E−12 |
| PGO345 | 3.20E−12 | FUE433 | 4.84E−12 |
| DRV470 | 3.61E−12 | LQM346 | 4.92E−12 |
| XUB802 | 3.32E−12 | GLK754 | 5.58E−12 |
| EPK959 | 3.66E−12 | VBE401 | 4.35E−12 |
| GYG794 | 3.90E−12 | LIZ707 | 8.35E−12 |
| DRO069 | 3.45E−12 | | |

### Binding Affinities to CD25 Measured by Surface Plasmon Resonance (SPR) Analysis

To provide optimal biased signaling to CD8 T cells, CD25 binding of IL-2 fusion proteins is preferably minimal. In order to understand if the CD25 binding site on the antibody IL-2 fusion proteins was accessible, binding to CD25 was assessed by surface plasmon resonance (SPR) analysis (BIAcore 3000, GE Healthcare, 33-1140587-3682). His-tagged recombinant CD25 was captured via TrisNTA Biotin on SA chips, and kinetic titration of IL-2 fusion proteins of the present invention was performed reaching concentrations up to 500 nM. IL-2 fusion proteins to Antibody A did not display binding to CD25 up to concentrations ≤500 nM. Antibody A binds IL-2 with high affinity on the CD25 binding site, thereby blocking IL-2 binding to the receptor CD25. Antibody B binds IL-2 with low affinity and allows IL-2 binding to CD25 only when fused to IL-2 with long linkers (>13 and 14 amino acids). Antibody C has no affinity for IL-2 and allows CD25 binding of its fused IL-2 with any linker length (Table 4 and 5).

pSTAT5 in Mouse Splenocytes (EC50 of Tregs, CD8, NK)

STAT5 phosphorylation was analyzed in murine splenocytes as downstream signaling of the IL-2R activation. In order to assess the in vitro selectivity of the antibody-IL-2 fusion proteins, pSTAT5 was measured in different cell populations after stimulation with Proleukin, or with the compounds of the present invention. Freshly isolated murine splenocytes from C57BL/6 mice were incubated with a dilution series of Proleukin or IL-2-antibody fusion protein, starting from 100 nM. The cells were immediately fixed and stained with surface markers (i.e., CD25, CD3, NK1.1, CD4, CD8). After permeabilization (Perm Ill buffer, BD Biosciences), intracellular staining was performed (FoxP3, pSTAT5) before acquisition through Flow Cytometry. % of pSTAT5+ cells of CD8+ T cells, NK cells and CD4⁺CD25⁺FoxP3⁺ Tregs were plotted for each compound against molar concentration of IL-2 antibody fusion protein. EC50 values were calculated with GraphPad Prism (Table 7). Potency on Treg cells of IL-2 fusion proteins to Antibody A is markedly decreased compared to Proleukin, while the EC50 values on NK and CD8 T-cells are comparable. Fusion proteins to antibody B show reduced potency on Treg cells compared to Proleukin, although the biased effect gets reduced with increasing linker length (compound BFC885). IL-2 fused to Antibody C signals with high potency to Treg cells, comparable to Proleukin. Affinity of the antibody to the permuted IL-2 is required in order to efficiently provide steric hinderance and thereby exclude the CD25 from the signaling complex (Table 7).

TABLE 7
EC50 of STAT5 activation of mouse splenocytes of IL-2-antibody
fusion proteins on NK cells, CD8 cells, and Treg cells.

| Compound | EC₅₀ NK | EC₅₀ CD8 | EC₅₀ Treg |
|---|---|---|---|
| name | cells (nM) | cells (nM) | cells (nM) |
| Proleukin | 1.4 | 4.1 | <0.016 |
| EAD406 | 5.2 | 15.7 | 22.0 |
| XFO227 | 2.7 | 8.0 | 10.6 |
| QTY065 | 1.2 | 4.6 | 5.91 |
| FJC828 | 1.9 | 5.8 | 8.4 |
| PGO345 | 2.3 | 6.6 | 7.6 |
| DRV470 | 2.4 | 7.9 | 10.3 |
| XUB802 | 5.8 | 17.1 | 12.1 |
| EPK959 | 1.9 | 16.0 | 16.0 |
| GYG794 | 1.5 | 18.6 | 4.8 |
| DRO069 | 0.9 | 6.8 | 6.6 |
| ECV200 | 0.9 | 6.9 | 3.7 |
| BFC885 | 2.0 | 26.4 | 0.9 |
| LPT269 | 1.7 | 32.0 | <0.016 |
| DXM339 | 1.3 | 18.6 | <0.016 |
| FUE433 | 1.2 | 15.7 | <0.016 |
| LQM346 | 1.8 | 25.4 | <0.016 |
| GLK754 | 2.5 | 70.5 | <0.016 |
| VBE401 | 34.3 | 32.2 | 2.6 |
| LIZ707 | 5.3 | 12.5 | 3.8 |

Applying the representative embedding procedures to IL-2-specific antibodies A or B resulted in fusion proteins with equally favorable IL-2 absence of CD25 binding (Table 5), stimulation of HEK Blue IL2 reporter cells (Table 6), and reduced potency in STAT5 phosphorylation on Treg cells compared to Proleukin (Table 7). This confirmed that by adjusting the orientation of the circularized IL-2 protein to conserve the natural binding orientation to an anti-IL-2 antibody to the cytokine, fusion to the heavy, or light chain variable region is feasible. A low affinity for IL-2 of antibody B (269 nM K_{D}) was sufficient to confer selective function of the resulting fusion protein. Peptide linkers of up to 20 amino acids in length retained desirable signaling qualities for the IL-2 fusion protein with Antibody A, though shorter peptides were preferred in combination with a lower affinity antibody. Fusion protein embedding IL-2CP in Antibody C did not exclude CD25 binding (even with 0-1 amino acid linkers), indicating that antibody affinity at least equivalent to Antibody B to IL-2 is required.

### Example 2: Generation of High Affinity Non-Blocking Anti-PD-1 Antibodies

Identification of Anti-hPD-1 Antibodies, Non-Competing with PD-1 Agonists

Three anti-human PD-1 antibodies that were described as non-competing with PD-1 antagonists or non-PD-L1 blocking were identified (Table 8). These antibodies were tested for competition with commercial PD-1 antagonistic antibodies by flow cytometry using 20× molar excess of competitor. Raji PD-1 expressing cells (Invitrogen) were incubated 30 minutes at 4° C. with serial dilutions of Pembrolizumab and Nivolumab, starting from 81 μg/ml (1:3 serial dilution). After washing the cells, antibodies in the first column of Table 8 labeled with biotin were added at a fixed concentration of 2 μg/ml. Bound antibodies were detected with Streptavidin-PE (Biolegend) and MFI levels of bound compound were compared to MFI in samples without competitor to determine the % signal inhibition. Background of samples incubated with Streptavidin-PE only was subtracted from all samples. Antibody XVT458 did not show any competition with any of the PD-1 antagonists tested.

Antibody ZJN296 showed partial competition with Pembrolizumab, with a reduced mean fluorescence intensity (MFI) compared to sample with no competitor of 47.4%. Nonetheless, since the signal did not show a concentration dependent decrease, the reduced MFI in the sample with 20× competitor concentration may be unrelated to the presence of Pembrolizumab. Nivolumab did not significantly decrease the signal of antibody ZJN296. Full competition between antibody OVL714 was observed with both Pembrolizumab and Nivolumab, indicating a possible common epitope on the antigen.

TABLE 8
Antibodies described as non-competing with PD-1 antagonists or PD-L1 and source.
Percent inhibition of binding signal to cells expressing human PD-1 when pre-coated
with 20x molar excess of the indicated commercial PD-1 antagonist antibodies.

| | | | Pembrolizumab | Nivolumab |
|---|---|---|---|---|
| Antibody | Species | Source | (% inhibition) | (% inhibition) |
| XVT458 | Humanized | (Clone NB01a) Fenwick C., J Exp Med, 2019 216(7): 1525 | 5.6 | 0 |
| ZJN296 | Mouse | (Clone SJL-565-4) Adler AS., mAbs, 2017 | 47.4 | 12.9 |
| OVL714 | Mouse | (Clone SJL-566-3) Adler AS., mAbs, 2017 | 99.5 | 99.3 |

### Re-Humanization and Affinity Maturation of Antibody XVT458

The humanized VH sequence of XVT458, composed of shuffled frameworks from different families of germline genes, was re-humanized in order to obtain a VH with harmonic germline (GH), to provide z0-XVT458 (SEQ ID NOS 053, 054). Re-humanization of VH of XVT458 was performed by CDR grafting onto human IGHV1-18*01 IGHJ6*01 framework, which showed highest homology to the framework regions of the parental construct. Mutations of residues of the vernier zone were inserted in order to observe impact on affinity to the hPD-1 antigen. Germline (GH wild type or with point mutations) and parental humanized (uVH) were combined with the humanized VL (uVL) in a full hIgG1 format (Table 9).

### Determination of K_{D}:

The binding of each to PD-1 was assessed by SPR (Biacore 8K, GE Healthcare). Anti-human Fc IgG (Jackson) was immobilized on amine reactive CM5 chips (GE Healthcare) using 1×HBS-EP+ running buffer (GE Healthcare). The test antibodies were captured at flow rate 10 μl/min with 30 s contact time. Anti-PD-1 to human PD-1 (hPD-1) (AcroBiosystems, PD1-H82E4) and cynomolgus primate (cyno) PD-1 (cynoPD-1) (AcroBiosystems, PD1-C5223) were run as analytes, diluted in 1×HBS-EP+ running buffer (GE Healthcare) with 240 s association and 600 s dissociation at flow rate 30 μl/min. Regeneration was carried out with 10 mM Glycine-HCl, pH 1.5 at flow rate 10 μL/min. This protocol is to be used, unless stated otherwise, in determination of Kd wherever used to define the K_{D} of antibodies specified herein, with adaptions where necessary with regard to the nature of the interaction partners.

TABLE 9
Derivatives of the anti PD-1 antibody XVT458 with newly
humanized VH and binding kinetics measured by SPR.

| Analyte | Ligand | VH | VL | kₐ (1/Ms) | k_{d} (1/s) | K_{D} (M) |
|---|---|---|---|---|---|---|
| hPD-1 Avi- | XVT458 | uVH | uVL | 8.30E+04 | 1.33E−03 | 1.60E−08 |
| Tag Biotin | z0-XVT458 | GH | uVL | 9.11E+04 | 1.47E−03 | 1.61E−08 |
| | z1-XVT458 | GH + V067A | uVL | 9.23E+04 | 1.46E−03 | 1.59E−08 |
| | z2-XVT458 | GH + T071V | uVL | 9.07E+04 | 1.27E−03 | 1.40E−08 |
| | z3-XVT458 | GH + T073K | uVL | 8.74E+04 | 1.42E−03 | 1.62E−08 |
| | z4-XVT458 | GH + V067A + T071V | uVL | 9.88E+04 | 1.31E−03 | 1.33E−08 |
| | z5-XVT458 | GH + V067A + T073K | uVL | 9.14E+04 | 1.45E−03 | 1.59E−08 |
| | z6-XVT458 | GH + T071V + T073K | uVL | 9.20E+04 | 1.33E−03 | 1.45E−08 |
| | z7-XVT458 | GH + V067A + T071V + T073K | uVL | 9.57E+04 | 1.36E−03 | 1.43E−08 |
| | z8-XVT458 | GH + V067A + T071V + T073K | mVL | No or weak binding | | |
| cynoPD-1 | XVT458 | uVH | uVL | 5.57E+04 | 3.73E−03 | 6.70E−08 |
| His | z0-XVT458 | GH | uVL | 6.32E+04 | 3.58E−03 | 5.67E−08 |
| | z1-XVT458 | GH + V067A | uVL | 6.27E+04 | 3.59E−03 | 5.73E−08 |
| | z2-XVT458 | GH + T071V | uVL | 5.72E+04 | 3.10E−03 | 5.42E−08 |
| | z3-XVT458 | GH + T073K | uVL | 5.74E+04 | 3.31E−03 | 5.77E−08 |
| | z4-XVT458 | GH + V067A + T071V | uVL | 6.33E+04 | 3.23E−03 | 5.10E−08 |
| | z5-XVT458 | GH + V067A + T073K | uVL | 5.86E+04 | 3.36E−03 | 5.73E−08 |
| | z6-XVT458 | GH + T071V + T073K | uVL | 5.84E+04 | 3.14E−03 | 5.38E−08 |
| | z7-XVT458 | GH + V067A + T071V + T073K | uVL | 5.89E+04 | 3.21E−03 | 5.44E−08 |

| | | | |
|---|---|---|---|
| z8-XVT458 | GH + V067A + T071V + T073K | mVL | No or weak binding |

Binding of the antibody candidates on cell surface expressed antigen was confirmed by flow cytometry. CHO-S hPD-1 cells were incubated with a 5-fold serial dilution of the indicated humanized antibodies. Antibody binding was detected with goat anti-human IgG-PE. MFI were plotted over the antibody concentration to obtain EC50 values. Binding kinetics on SPR, and FACS EC50 binding values and maximal MFI values, indicated antibodies bound to hPD-1 expressing cells, except z8-uIgGKV326 (Tables 9 and 10).

TABLE 10
Top MFI values and EC50 values of anti-PD-1 antibodies
binding to hPD-1 expressed on CHO-S cells.

| Ligand | Top MFI | FACS EC50 nM |
|---|---|---|
| XVT458 | 45100 | 8.60 |
| z0-XVT458 | 45185 | 5.74 |
| z1-XVT458 | 45235 | 5.85 |
| z2-XVT458 | 45528 | 4.15 |
| z3-XVT458 | 46192 | 5.47 |
| z4-XVT458 | 46726 | 4.54 |
| z5-XVT458 | 46173 | 4.51 |
| z6-XVT458 | 46791 | 3.93 |
| z7-XVT458 | 45991 | 3.88 |
| z8-XVT458 | 0 | NA |
| IgG1 isotype control | 0 | NA |

The humanized antibody z2-XVT458 (SEQ ID NOS 055, 056) was affinity matured by inserting random mutations in the CDRs (parsimonious mutagenesis method). Every position in the antibody VH and VL-CDRs was mutated by PCR using a mutagenic primer containing a degenerate codon NNS at a specific CDR position, introducing a combination of all 20 amino acids. The library of single mutants was screened by scFv capture ELISA using a redundancy factor of 4 (on 96-well plate er position). Clones displaying ≥2× ELISA signal compared to the wild-type scFv were sequenced, and clones with unique sequences were re-grown, re-screened by capture ELISA and ranked by dose-dependent ELISA on human and cyno antigens, and by FACS using human antigen expressing cells. Affinity improving mutations were used to design a combinatorial library using the Kunkel method. The library was screened by scFv capture ELISA using the redundancy factor of 4 (4× number of combinations). Clones displaying ≥2× ELISA signal compared to the wild-type scFv were sequenced, and clones with unique sequences were re-grown, re-screened by capture ELISA and ranked by dose-dependent ELISA on human and cyno antigens, and by FACS using human antigen expressing cells. Primary screening by saturated mutagenesis of antibody CDRs identified 25 mutants at 10 CDR amino acid positions that showed capture ELISA signal at least 2-fold higher than wild-type on hPD-1. 9 mutants showed improved binding to both recombinant hPD-1 and cynoPD-1, and hPD-1 on CHO-S cells. A combinatorial library was generated with these 9 mutants yielding 16 combinatorial variants with improved affinity to recombinant and cell-expressed antigen. From the base sequence of z2-XVT458 (variable domains SEQ ID NOs 055, 056), 6 subclones, z2-XVT458 m1-m6 (SEQ ID NOS 057 to 068), were selected for IgG conversion. In addition to the CDR mutations, the sequences of z2-XVT458-m1, z2-XVT458-m3 and z2-XVT458-m6 contain a G57D mutation just after the CDRL2, which may contribute to the increased affinity of these clones. The selected clones in IgG format showed a significant affinity improvement to recombinant hPD-1, cynoPD-1 and hPD-1 on cells. Compared to parental z2-XVT458, these clones (z2-XVT458m1-m6) also displayed 10-84 folds improvement in k_{off} rate as measured by SPR on both hPD-1 and cynoPD-1 (Table 11; corresponding Seq ID NOs 057-068). For SPR the same method as above was used.

TABLE 11
Parental XVT458, z2-XVT458 and subclone affinity
matured mutants in IgG format. Binding affinities
measured by flow cytometry, ELISA and SPR.

| | ELISA Binding | | | |
|---|---|---|---|---|
| | hPD-1 | | cynoPD-1 | |
| Abs | EC50 (nM) | Max OD | EC50 nM(nM) | Max OD |
| XVT458 | 1.04 | 2.85 | 0.5 | 3.21 |
| XVT458-z2 | 0.71 | 2.91 | 0.4 | 3.32 |
| XVT458-z2-m1 | 0.083 | 3.39 | 0.087 | 3.58 |
| XVT458-z2-m3 | 0.089 | 3.37 | 0.094 | 3.54 |
| XVT458-z2-m5 | 0.051 | 3.42 | 0.056 | 3.58 |
| XVT458-z2-m6 | 0.064 | 3.49 | 0.071 | 3.69 |
| XVT458-z2-m4 | 0.079 | 3.3 | 0.098 | 3.54 |
| XVT458-z2-m2 | 0.076 | 3.34 | 0.086 | 3.56 |
| NC.hIgG1 | >50 | 0.05 | >50 | 0.07 |

| | FACS Binding | | | |
|---|---|---|---|---|
| | CHO-S hPD-1 | | CHO-S Cyno-PD-1 | |
| Abs | EC₅₀ (nM) | Max MFI | EC₅₀ (nM) | Max MFI |
| XVT458 | 6.73 | 39900 | NA | 52 |
| XVT458-z2 | 5.86 | 40700 | NA | 109 |
| XVT458-z2-m1 | 1.5 | 41000 | NA | 70 |
| XVT458-z2-m3 | 1.34 | 41100 | NA | 124 |
| XVT458-z2-m5 | 0.84 | 42400 | NA | 335 |
| XVT458-z2-m6 | 1.56 | 39500 | NA | 59 |
| XVT458-z2-m4 | 0.96 | 43400 | NA | 258 |
| XVT458-z2-m2 | 1.15 | 42800 | NA | 367 |
| NC.hIgG1 | NA | 184 | NA | 206 |
| | SPR | | | |
| | hPD-1 | | | |
| Abs | ka (1/Ms) | kd (1/s) | K_{D} (M) | ka (1/Ms) |
| XVT458 | 9.48E+04 | 1.22E−03 | 1.28E−08 | 6.90E+04 |
| XVT458-z2 | 9.92E+04 | 1.24E−03 | 1.25E−08 | 7.30E+04 |
| XVT458-z2-m1 | 2.22E+05 | 3.28E−05 | 1.48E−10 | 1.85E+05 |
| XVT458-z2-m3 | 1.58E+05 | 6.57E−05 | 4.17E−10 | 1.38E+05 |
| XVT458-z2-m5 | 1.24E+05 | 9.77E−05 | 7.87E−10 | 1.11E+05 |
| XVT458-z2-m6 | 2.31E+05 | 1.01E−04 | 4.39E−10 | 2.13E+05 |
| XVT458-z2-m4 | 1.42E+05 | 1.73E−04 | 1.22E−09 | 1.34E+05 |
| XVT458-z2-m2 | 1.53E+05 | 1.90E−04 | 1.24E−09 | 1.27E+05 |
| NC.hIgG1 | / | / | / | / |

### Humanization of Antibody ZJN296

ZJN296 was humanized by CDR grafting using germlines with highest identity with the murine framework (IGKV1-33*01 IGKJ2 and IGHV1-18*01 IGHJ6*01). To increase the likelihood of retaining the binding affinity to the target antigen, 8 additional humanized VL-genes and 2 humanized VH-genes were designed by mutating human amino acids back to mouse. Such mutations may preserve the original conformation of VH and VL CDR loop and antigen binding if this loop makes contact with the antigen (Table 12).

TABLE 12
Antibody variants of humanized ZJN296, with
mutations inserted in humanized ZJN296-0 VH
(GH, SEQ ID NO 069) and VL (SEQ ID NO 070).

| ID | VH | VL |
|---|---|---|
| ZJN296 | mVH | mVK |
| ZJN296-0 | GH | GL |
| ZJN296-1 | GH + V002A | GL |
| ZJN296-2 | GH + T071V | GL |
| ZJN296-3 | GH + T073R | GL |
| ZJN296-4 | GH + V002A + T071V | GL |
| ZJN296-5 | GH + V002A + T073R | GL |
| ZJN296-6 | GH + T071V + T073R | GL |
| ZJN296-7 | GH + V002A + T071V + T073R | GL |
| ZJN296-8 | GH | GL + S060D |
| ZJN296-9 | GH + V002A | GL + S060D |
| ZJN296-10 | GH + T071V | GL + S060D |
| ZJN296-11 | GH + T073R | GL + S060D |
| ZJN296-12 | GH + V002A + T071V | GL + S060D |
| ZJN296-13 | GH + V002A + T073R | GL + S060D |
| ZJN296-14 | GH + T071V + T073R | GL + S060D |
| ZJN296-15 | GH + V002A + T071V + T073R | GL + S060D |
| ZJN296-16 | GH + V002A + V067A + T071V + T073R | GL + S060D |
| ZJN296-17 | mVH | GL + S060D |
| ZJN296-18 | GH + V002A + V067A + T071V + T073R | mVL |

Recombinant humanized antibody variants of ZJN296 were expressed as human IgG1 in HEK293 cells. For SPR analysis (Biacore 8K, GE Healthcare) anti-human Fc IgG (Bethyl, A80-304P) was immobilized on CM5 chips (Cytiva) using 1×HBS-EP+ running buffer. Humanized anti-PD-1 antibodies were captured on the chip. hPD-1 and cynoPD-1 were run as analytes 1×HBS-EP+ running buffer at a flow rate of 30 μL/min for an association phase of 180 s, followed by 400 s dissociation. 10 mM glycine (pH 1.5) as regeneration buffer was injected to flow cells following every dissociation phase.

Based on SPR binding kinetics on human and cyno antigen and binding data to hPD-1 expressed on cells tested with supernatants, 5 best candidates were expressed recombinantly and a full SPR kinetics was performed (Table 13). ZJN296-0 and ZJN296-6 were selected as candidates since ZJN296-0 has minimum back mutations and comparable binding potency compared with the chimeric antibody, and ZJN296-6 has the best binding characteristics.

TABLE 13
SPR kinetics results of humanized purified ZJN296 derived antibodies

| | Binding to Human PD1 | | | Binding to Cyno PD1 | | |
|---|---|---|---|---|---|---|
| Sample Name | kₐ (1/Ms) | k_{d} (1/s) | k_{D} (M) | kₐ (1/Ms) | k_{d} (1/s) | k_{D} (M) |
| ZJN296 | 7.71E+04 | 7.37E−04 | 9.56E−09 | 8.93E+04 | 1.15E−02 | 1.28E−07 |
| ZJN296-0 | 5.69E+04 | 4.95E−04 | 8.70E−09 | 6.15E+04 | 6.41E−03 | 1.04E−07 |
| ZJN296-2 | 5.65E+04 | 3.24E−04 | 5.72E−09 | 5.96E+04 | 4.33E−03 | 7.27E−08 |
| ZJN296-3 | 5.61E+04 | 3.69E−04 | 6.57E−09 | 4.76E+04 | 5.11E−03 | 1.07E−07 |
| ZJN296-6 | 6.26E+04 | 2.01E−04 | 3.22E−09 | 6.36E+04 | 2.79E−03 | 4.39E−08 |

### Non-Blocking Antibody Hybridoma Screening

High affinity binding antibodies specific for human PD-1 (hPD-1), cross-reactive with cynomolgus monkey PD-1 (cynoPD-1), but not blocking PD-L1 or PD-1 antagonist antibodies were generated by immunization of humanized mice (AlivaMab® mice) and hybridoma technology. Two separate immunization campaigns (with 10 Kappa-Lambda mice in first campaign, 4 Kappa and 4 Lambda in second campaign) were performed by immunization with a combination of h/cyPD-1. Primary functional screening was performed using hPD-1 and cyPD-1 transfected HEK293 cells. Binding was assessed by flow cytometry. 31 plates of 384 wells were screened in the first campaign, and 15 plates of 384 wells were screened in the second campaign. In the second screen selected clones were initially tested from supernatants for competition with Nivolumab and Pembrolizumab, testing binding on antigen expressing cells by flow cytometry in presence of the competitor antibodies and detecting with an anti-mouse IgG antibody. Non-competitors were determined as MFI(+ competitor)/MFI(no competitor) >0.6. Only the non-competing clones, that showed high affinity binding on BLI (K_{D}<1 nM) were expanded and purified. Out of all the screened clones, 7 clones fitting the desired binding profile were identified (Table 14, SEQ ID NOs 071 to 084 VH and VL of antibodies that were used with CH_{1,2,3} and corresponding CL(kappa) or CL(lambda)). Table 14 summarizes the binding properties to human and cyno PD-1. EC50 binding to HEK293 cells transfected with hPD-1 and cyPD-1 were measured by flow cytometry. Binding kinetics to the recombinant antigen was measured by BLI. Association (220s) and dissociation (480s) of a dilution series of recombinant hPD-1 and cynoPD-1 (100, 25, 6.25 nM, AcroBio) were measured on the antibody-coated biosensors (anti-human-IgG CH1 Biosensor, ForteBio) Octet-System (Octet RED, ForteBio). K_{D} values were obtained by fitting the kinetics data with the ForteBio Data Analysis Software (8.2). PBS was used as assay buffer (PBS 10 mM Phosphate, 150 mM NaCl—pH 7.4). For dissociation the biosensors were dipped into kinetic assay buffer (PBS 10 mM Phosphate, 150 mM NaCl—0.1% BSA—0.02% Tween—pH 7.4). Sequencing of the final clones revealed that clones 21A08, 22F13 and 25120 used the same HC/LC V-regions and have the same CDR3s. Clones 20H02, 39F23, 40B20 and 56H02 showed multiple development liabilities in the CDR regions. Clone 21A08 was selected as the best clone for its affinity and purity from SEC-HPLC compared to the other sibling clones.

TABLE 14
FACS EC50 binding to hPD-1 or cynoPD-1 expressing HEK293
cells, SPR binding kinetics on hPD-1 and cynoPD-1.

| | FACS EC50 (M) | | PD1-HIS | | | cynoPD-1-HIS | | |
|---|---|---|---|---|---|---|---|---|
| clone | 293:huPD1 | 293:cyPD1 | KD (M) | kdis(1/s) | kon(1/Ms) | KD (M) | kdis(1/s) | kon(1/Ms) |
| 21A08 | 2.43E−10 | 1.95E−10 | 9.27E−12 | 6.39E−07 | 6.90E+04 | 1.24E−09 | 8.25E−05 | 6.67E+04 |
| 22F13 | 2.00E−10 | 1.07E−10 | 1.41E−09 | 1.32E−04 | 9.40E+04 | 8.45E−09 | 8.38E−04 | 9.92E+04 |
| 25I20 | 1.79E−10 | 3.10E−10 | 1.15E−09 | 1.01E−04 | 8.75E+04 | 8.38E−09 | 7.83E−04 | 9.34E+04 |
| 20H02 | 5.03E−10 | 2.31E−10 | 2.43E−09 | 2.21E−04 | 9.09E+04 | 6.39E−09 | 4.63E−04 | 7.25E+04 |
| 39F23 | 1.89E−11 | 2.89E−11 | 1.03E−09 | 2.53E−04 | 2.44E+05 | 1.30E−09 | 2.94E−04 | 2.27E+05 |
| 40B20 | 1.62E−10 | 9.58E−11 | 9.61E−09 | 7.73E−04 | 8.05E+04 | 7.51E−09 | 5.05E−04 | 6.73E+04 |
| 56H02 | 9.21E−11 | 8.81E−11 | 2.28E−10 | 3.79E−05 | 1.66E+05 | 2.62E−10 | 3.67E−05 | 1.40E+05 |

In order to remove an N-glycosylation site present in the CDR-L1, mutations were inserted in the CDR-L1 of 21A08. The first asparagine (Asp, N) in CDR-L1, was replaced by a serine (Ser, S), Glutamine (Gln, Q), or Alanine (Ala, A). Binding of the 21A08 clone variants (21A08S, 21A08Q and 21A08A) was tested by BLI and compared to the wt clone using supernatants of transiently transfected HEK293 cells, and a positive control (Pembrolizumab).

TABLE 15
Antibody clone 21A08 and deglycosylated variants with
binding affinity (KD) and dissociation constant (Koff).

| Clone name | Mutations LCDR1 | KD huPD-1 (M) | Koff 1/s |
|---|---|---|---|
| 21A08 | — | 2.17E−09 | 1.40E−04 |
| 21A08S | N > S | 2.47E−09 | 1.84E−04 |
| 21A08Q | N > Q | 3.39E−09 | 2.67E−04 |
| 21A08A | N > A | 1.81E−09 | 1.81E−04 |
| Pembrolizumab | | 6.29E−09 | 1.61E−03 |

In addition, a deamidation site present in the CDR-L1 was removed by inserting the mutations listed in Table 16. Binding kinetics of 21A08A and the deamidation site removal mutants were measured by SPR. For SPR analysis (Biacore 8K, GE Healthcare) anti-human Fc IgG (Bethyl, A80-304P) was immobilized on CM5 chips (Cytiva) and the test anti-PD-1 antibodies were captured using 1×HBS-EP+ running buffer at flow rate 10 μL/min. hPD-1 and cynoPD-1 (His-tagged) were run as analytes diluted in 1×HBS-EP+ running buffer at a flow rate of 30 μL/min for an association phase of 250 s, followed by 3600 s dissociation. 10 mM glycine (pH 1.5) as regeneration buffer was injected to flow cells following every dissociation phase. All clones lacking the N-glycosylation site retained binding to PD-1 (Table 15). None of the mutations in the CDR-L1 compromised the binding kinetics to hPD-1 or cynoPD-1 significantly.

TABLE 16
Antibody clone 21A08A and variants with deamidation site
removed, binding affinity and dissociation constant

| Clone | Mutation | ka hPD-1 | kd hPD-1 | KD hPD-1 | ka cPD-1 | kd cPD-1 | KD cPD-1 |
|---|---|---|---|---|---|---|---|
| name | LCDR1 | (1/Ms) | (1/s) | (M) | (1/Ms) | (1/s) | (M) |
| 21A08A | — | 6.3E+04 | <1.0E−05 | <1.6E−10 | 5.6E+04 | 9.6E−05 | 1.7 E−09 |
| 21A08Ap1 | NS > QS | 5.7E+04 | 2.9E−05 | 5.0 E−10 | 5.2E+04 | 1.6E−04 | 3.0 E−09 |
| 21A08Ap2 | NS > SS | 6.4E+04 | 4.2E−05 | 6.6 E−10 | 5.9E+04 | 1.9E−04 | 3.2 E−09 |
| 21A08Ap3 | NS > NA | 6.0E+04 | 3.5E−05 | 5.8 E−10 | 5.5E+04 | 2.5E−04 | 4.67E−09 |

### Example 3: Binding Profile of Non-Blocking Anti-PD-1 Antibodies

Assessment of the binding profile of non-blocking anti-PD-1 antibodies was then made using a human plasma membrane protein cell array. The Retrogenix Cell Microarray Technology platform screened the anti-PD-1 antibody candidates for cross-reactivity binding with non-target proteins. The test antibodies were each screened for binding against human HEK293 cells, individually expressing 6018 full-length human plasma membrane proteins, secreted and cell surface-tethered human secreted proteins plus a further 397 human heterodimers. In a pre-screen 2 μg/mL of each test antibody or PBS only was added to slides of fixed un-transfected HEK293 cells. Binding to un-transfected cells was assessed using an AlexaFluor647 labelled anti-human IgG Fc detection antibody (AF647 anti-hIgG Fc), followed by fluorescence imaging. In the pre-screen with antibody XVT458-z2-m5 a high background was detected. A second pre-screen was repeated with 0.5 mg/ml, which resulted in background reduction. For library screening 6018 expression vectors, encoding both ZsGreenl and a full-length human plasma membrane protein or a cell surface-tethered human secreted protein, were individually arrayed in duplicate across 17 microarray slides (‘slide-sets’). In addition, vectors encoding a further 397 human heterodimers were co-arrayed across a further microarray slide. Human HEK293 cells were used for reverse transfection/expression. Test antibody XVT458-z2-m5 was added to each of the 18 slide-sets after cell fixation giving a final concentration of 0.5 μg/mL, the remaining 9 test antibodies were added at final concentration of 2 μg/mL. Detection of binding was performed by using the same fluorescent secondary antibody as used in the pre-screen (AF647 anti-hIgG Fc). Fluorescent images were analyzed and quantitated (for transfection) using ImageQuant software (GE healthcare, Version 8.2). A protein ‘hit’ is defined as a duplicate spot showing a raised signal compared to background levels. This is achieved byvisual inspection using the images gridded on the ImageQuantsoftware. Hits were classified as ‘strong, medium, weak or very weak’, depending on the intensity of the duplicate spots. The screen revealed 29 library hits. After removing 12 interactions which were observed with a test antibody and the control treatment and therefore designated as non-specific, a further 7 low confidence interactions, 10 specific interactions for the test antibodies were identified (Table 17). All test antibodies showed a single specific interaction with PD-1 (PDCD1). Test antibodies ZJN296-0, ZJN296-6, XVT458-z2-m1, XVT458-z2-m2, XVT458-z2-m4, XVT458-z2-m5 all showed medium to weak interactions with other target proteins. Clones 21A08Ap1 (SEQ ID NO VH 085, VL 086), 21A08Ap2 (SEQ ID NO VH 085, VL 087), XVT458-z2-m3 (SEQ ID NO 061, 062), and XVT458-z2-m6 (SEQ ID NO 067, 068) showed single specific interaction to the target of interest.

TABLE 17
Hits from the Retrogenix cell microarray screen. Protein types are Plasma membrane (PM), Secreted (S), Tethered
Secreted (TS), Heterodimer (HD) or Evidence of plasma membrane (M). Hits no., above very weak intensity.

| | | Hit no. | | | | | |
|---|---|---|---|---|---|---|---|
| | | Gene ID | | | | 4 | |
| | | Protein Type | 1 | 2 | 3 | APP | 5 |
| | | Ref seq | PDCD1 | RTN4RL2 | PROM2 | PM | SV2A |
| | | isoform (for [symbol] | PM | PM | PM | Isoform | PM |
| | | Hit no. | single form | Isoform 1 | Isoform 1 | APP751 [symbol] | Isoform 1 |
| | | Gene ID | 6 | 7 | 8 | 9 | 10 |
| | | Protein Type | DCC | RARRES2 | IGSF1 | CD248 | NLGN1 |
| Sample Id and dose | Hits | Ref seq | PM | TS | PM | PM | PM |
| Sample Id and dose | Hits | isoform (for [symbol] | single form | single form | Isoform 1 | Isoform 1 | Isoform 2 |
| 21A08Ap1 (2 μg/mL) | 1 | | | | | | |
| 21A08Ap2 (2 μg/mL) | 1 | | | | | | |
| ZJN296-0 (2 μg/mL) | 2 | | | | | | |
| ZJN296-6 (2 μg/mL) | 2 | | | | | | |
| XVT458-z2-m1 (2 μg/mL) | 2 | | | | | | |
| XVT458-z2-m2 (2 μg/mL) | 6 | | weak | weak | | | v. weak |
| XVT458-z2-m3 (2 μg/mL) | 1 | | | | | | |
| XVT458-z2-m4 (2 μg/mL) | 4 | | | | weak | weak | weak |
| XVT458-z2-m5 (0.5 μg/mL) | 2 | | | weak | | | v. weak |
| XVT458-z2-m6 (2 μg/mL) | 1 | | | | | | |
| PBS (secondary only)* | | | | | | | |

[symbol]

indicates data missing or illegible when filed

### Example 4: Non-Competitive Binding of Anti-PD-1 Antibodies with PD-L1 Antagonists and PD-L1

Competition on hPD-1 Expressing Cells

Antibodies 21A08Ap1 and 21A08Ap2 were tested for competition with commercial PD-1 antagonistic antibodies by flow cytometry. Jurkat PD-1 expressing cells were incubated 30 minutes at 4° C. with a serial dilution of Pembrolizumab and Nivolumab, starting from 10 pM (1:3 serial dilution). Without washing the cells antibodies 21A08Ap1 and 21A08Ap2 labeled with biotin were added at a fixed concentration of 100 nM. Bound antibodies were detected with Streptavidin-PE (Biolegend) and MFI levels of bound compound were compared to MFI in samples without competitor to determine the % signal inhibition. Background of samples incubated with Streptavidin-PE only was subtracted from all samples. Inhibition was minimal, under 20%, showing neither Pembrolizumab nor Nivolumab significantly decreased the signal of antibodies 21A08Ap1 and 21A08Ap2 (Table 18).

TABLE 18
Competition on hPD-1 expressed on cells with commercial
PD-1 antagonists, Pembrolizumab and Nivolumab. Flow
cytometry % signal inhibition of the tested antibody
using 100x molar excess of competitor.

| | |
|---|---|
| Antibody | Signal inhibition in % with 100x molar excess |

| name | Pembrolizumab | Nivolumab |
|---|---|---|
| 21A08Ap1 | 11.1 | 7.3 |
| 21A08Ap2 | 4.4 | 0 |

Simultaneous Binding of hPD-1 to 21A08Ap1 and PD-L1

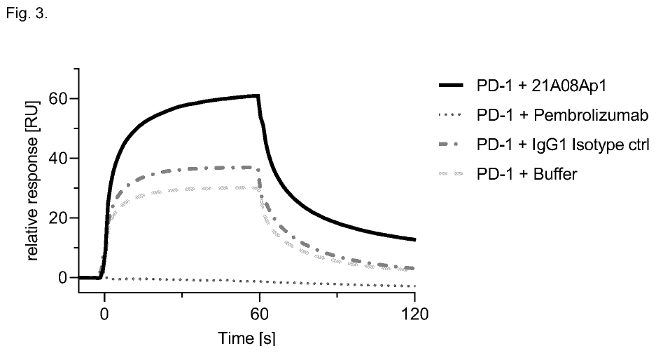

Simultaneous binding of PD-1 (Fc-Tag) to anti-PD-1 antibodies and PD-L1 (His-Tagged, Acrobiosystems, #H52H3) was assessed by SPR analysis using the Biacore T200 (Cytiva, #28975001). PD-L1 was captured on an anti-His-CM5 chip (Cytiva, chip: #29104988, His Capture Kit: #28995056). 1 μM of PD-1 was premixed with 5 μM of antibody (Pembrolizumab, IgG1 isotype control antibody, or 21A08p1) or running buffer (Xantec, HBSTE: B HBSTE10) for at least 30 min before binding analysis (association and dissociation 60s each). Data was analyzed using the Biacore Insight Evaluation Software (Cytiva, V4.0.8 #29310606). Double reference subtraction was performed (surface without ligand and running buffer injection). FIG. 3 reports the Biacore sensograms. No binding (as change in RU) was observed for PD-1 pre-mixed with Pembrolizumab, confirming that Pembrolizumab blocks the PD-L1 binding site of PD-1. By contrast, the complex of PD-1 with the antibody 21A08Ap1 was still able to bind to PD-L1, giving a signal of approximately 60 RU. The controls of PD-1 alone and PD-1+IgG1 isotype control showed an association signal of approximately 30 RU.

### Example 5: Rational Design of Non-Blocking PD-1 Targeted IL-2 Fusion Proteins

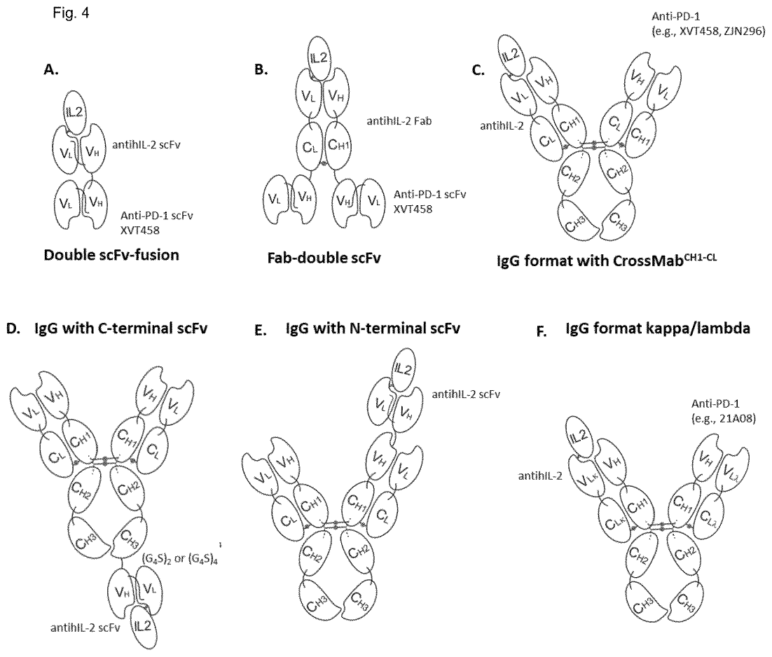

Proof-of-concept compounds were designed and characterized to determine the most favorable format for adding a non-blocking PD-1 targeting moiety to a CD122/CD132 dimeric receptor biased IL-2-anti-IL-2 antibody fusion moiety. The anti-PD-1 antibodies XVT458 or ZJN296 (Table 8) were combined with Antibody A—IL-2 fusion protein (QTY065, SEQ ID NO 051, 052) to generate bispecific compounds delivering specifically CD122-CD132 biased IL-2 to PD-1 expressing cells. Five formats were designed different in size and valency to the PD-1 antigen (FIG. 4, Table 19). The double-scFv fusion comprises the anti-PD-1 antibody XVT458 in scFv format, i.e. the VH and VL domains fused by a 15 amino acid Glycine(G)-Serine(S) linker, fused to the QTY065 IL-2-antibody fusion scFv by a G4S linker (FIG. 4A). The Fab-double scFv consists of the QTY065 antibody-IL-2 fusion as Fab fragment (IL-2-VL-CL and VH-CH1) fused at each C-terminus to two anti-PD-1 scFv fragments of the XVT458 antibody (by their N-terminus VH-(G4S)3-VL, FIG. 4B). The IgG CrossMab format is a heterotetrameric human IgG1, with HC1 and LC1 from the anti-PD-1 antibodies XVT458 or ZJN296 and HC2 and LC2 from antibody-IL-2 fusion QTY065 (FIG. 4C). Knob into hole mutations (Y407T on HC1 and T366Y on HC2) were used to ensure correct heavy chain pairing. To enhance correct light chain pairing the CL and CH1 domains of HC1 and LC1 were swapped (WO2009080253A1). For Fc silencing of the IgG1 Fc region the mutations L234A, L235A, P329G (WO2012130831A1) were inserted on both HC1 and HC2. The IgG-scFv fusion proteins include the antibody XVT458 as full human IgG1 (consisting of HC1, identical LCs, HC2) having the HC2 fused on either its N-terminus (4E) or C-terminus (4D) to the IL-2-antibody fusion QTY065 as scFv (i.e., IL-2-VL_{AntibodyA}-(G₄S)₃-VH_{AntibodyA}-(G₄S)₂-HC2_{XVT458} or HC2_{XVT458}-(G₄S)_{2/4}-IL-2-VL_{AntibodyA}-(G₄S)₃-VH_{AntibodyA}). Pairing to HC1 is enhanced by the knob into hole mutations (same as CrossMab) and Fc silencing by L234A, L235A, P329G mutations. The bispecific antibodies were produced in Expi297 cells as described for IL-2 fusion proteins described above.

The functionality of the bispecific antibody was assessed with a sandwich ELISA that relies on its binding to the target antigen (coated hPD-1) and the integrity of the fused IL-2 via a secondary antibody (anti-IL-2 clone 5344). 60 nM of hPD-1 (ECD-His, produced in house) was coated on a Maxisorp plate (Nunc) overnight at 4° C. and blocked with 5% BSA in PBS. Bispecific antibodies were added in a serial dilution in assay buffer and detected with biotinylated-5344 and Streptavidin HRP (BD Pharmingen, 554066). Absorbance signal after adding TMB was read with a plate reader (Spectramax ID3) at 450 nm. EC50 values were determined by blotting absorbance against concentration (Graphpad Prism, sigmoidal curve fit, 4 PL, X-axis is log). All compounds showed binding on both bispecific moieties; formats with bivalent binding to hPD-1 showed lower EC50 values compared to the monovalent formats (Table 19). Binding to hPD-1 expressed on the surface of Jurkat-PD-1 cells was confirmed by flow cytometry (Table 19). All formats retained binding to hPD-1. As expected, compounds bearing two PD-1-binding domains showed increased binding to hPD-1 due to avidity effects.

TABLE 19
Anti-PD-1, anti-hIL-2-IL-2 bispecific antibodies in various formats,
with valency, molecular weight (Mw), EC50 to hPD-1 measured
by ELISA and binding to hPD-1 expressed on Jurkat-hPD-1 cells
measured by flow cytometry (MFI fold over background).

| | | | | Anti- | | | |
|---|---|---|---|---|---|---|---|
| | | | Binding | hIL-2/ | | | hPD-1 |
| Compound | Anti PD-1 | | arms to | IL-2 | Mw | EC50 | binding |
| ID | antibody | Format | hPD1 | arms | (kDa) | (nM) | (fold) |
| HRL470 | XVT458 | Double scFv | 1 | 1 | 67.7 | 1.24 | 41.9 |
| | | fusion | | | | | |
| VNP090 | XVT458 | Fab- double scFv | 2 | 1 | 116 | 0.11 | 19.9 |
| FQQ111 | XVT458 | IgG format with | 1 | 1 | 161 | 1.16 | 46.5 |
| | | CrossMab^{CH1-CL} | | | | | |
| ONG682 | ZJN296 | IgG format with | 1 | 1 | 161 | 0.87 | 20.1 |
| | | CrossMab^{CH1-CL} | | | | | |
| YMI345 | XVT458 | IgG with N- | 2 | 1 | 187 | 0.12 | 54.8 |
| | | terminal scFv | | | | | |
| CUM013 | XVT458 | IgG with C- | 2 | 1 | 188 | 0.11 | 47.3 |
| | | terminal scFv, | | | | | |
| | | (G4S)4 linker | | | | | |
| QWT744 | XVT458 | IgG with C- | 2 | 1 | 187 | 0.12 | 42.7 |
| | | terminal scFv, | | | | | |
| | | (G4S)2 linker | | | | | |

### Example 6: In Vitro and In Vivo Cellular Selectivity

In order to assess the functionality of the anti-IL-2/IL-2 fusion protein arm of the bispecific compounds, STAT5 phosphorylation was analyzed in murine splenocytes as downstream signaling of the IL-2R activation. pSTAT5 was measured in different cell populations after stimulation with Proleukin, or with one of the bispecific compounds. Mouse splenocytes were incubated with a serial dilution of bispecific compounds diluted in RPMI+10% FBS. Starting concentration 100 nM, dilution rate 1:5 obtaining 6 total concentrations. Samples were incubated at 37 00 for 15 minutes and fixed immediately afterwards by adding same volume of cytofix buffer (BD Biosciences, Cat #554655) per sample and incubating samples for 10 minutes at 37 00. After fixation cells were stained with BV421 rat anti-mouse 0025 (clone P061, BD Biosciences, 0.5 L/samples), BV650 hamster anti-mouse CD3e (clone 145-2011, BD Biosciences, 1 L/samples), BV711I mouse anti-mouse NK1.1 (clone P0136, BD Biosciences, 0.33 μL/samples) for 30 minutes at room temperature. Subsequently, cells were permeabilized using Perm Buffer Ill (BD Biosciences, Cat #558050) incubating for 10 minutes on ice. A second staining included PE-CF594 rat anti-mouse 004 (clone RM4-5, BD Biosciences, 0.25 μL/samples), APC-780 rat anti-mouse CD8b (clone H35-17.2, eBioscience, 0.167 μL/samples), AF488 rat anti-mouse FoxP3 (clone FJK-16, eBioscience, 0.5 μL/samples), AF647 mouse anti-mouse pSTAT5 (clone pY694, BD Bioscience, 20 μL/samples). All samples were acquired with the software SpectroFlo® on the Cytek® Aurora. The .fcs files were analyzed with FlowJo_v10.6.2. EC50 of % pSTAT5+ values were obtained by sigmoidal 4-PL fitting. % of pSTAT5+ cells were plotted for each cell population and each tested compound against the log concentration (M).

The obtained EC50 values are shown in Table 20. All formats, except the IgG C-terminal scFv bispecifics, showed comparable (or max. 10 fold lower) potency in inducing STAT5 phosphorylation in NK and CD8 T cells compared to Proleukin (Table 20). EC50 values for QWT744 and CUM013 could not be determined, as the % pSTAT5 positive cells with the highest concentration of compound tested (100 nM) did not reach maximal levels. All compounds showed reduced potency in inducing pSTAT5 in Treg cells compared to Proleukin, indicating that signaling through the high affinity trimeric IL-2R is hindered in vitro (Table 20).

TABLE 20
Bispecific compounds, QTY065, and Proleukin EC50 values
for pSTAT5 positive cells in parental NK, CD8, and Treg
cells measured by flow cytometry in mouse splenocytes.

| | EC50 pSTAT5 on | EC50 pSTAT5 on | EC50 pSTAT5 on |
|---|---|---|---|
| Compound ID | NK cells (nM) | CD8 cells (nM) | Treg cells (nM) |
| Proleukin | 2.51 | 11.92 | <0.02 |
| QTY065 | 2.88 | 17.93 | 4.21 |
| HRL470 | 4.04 | 27.45 | 1.18 |
| VNP090 | 14.67 | 26.81 | 10.94 |
| FQQ111 | 27.34 | 34.1 | 6.55 |
| YMI345 | 23.77 | 29.6 | 13.67 |
| CUM013 | >100.00 | >100.00 | >100.00 |
| QWT744 | >100.00 | >100.00 | >100.00 |
| ONG682 | 14.08 | 23.87 | 12.72 |

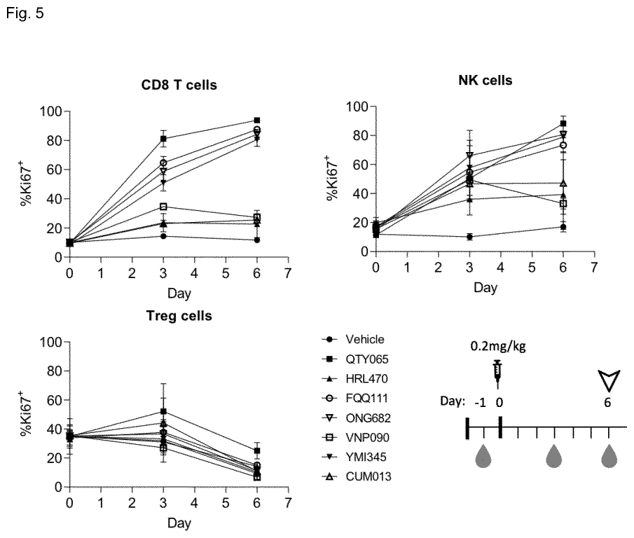

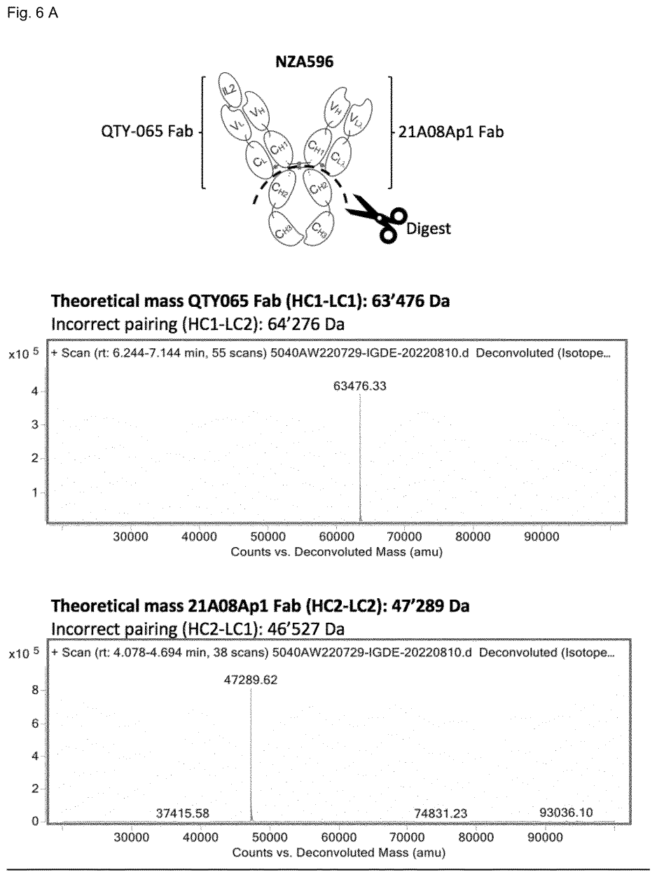

To confirm selectivity towards CD122-CD132 expressing cells in vivo, bispecific compounds were administered i.v. at 0.2 mg/kg to wt C57BL/6 mice. Blood samples were taken before the injection and on days 3 and 6 after compound administration. Cell proliferation was analyzed by flow cytometry, detecting Ki67+ cells in CD8 T cells, NK cells and Treg cells (FIG. 5). C57BL/6 mice were injected i.v. with a single dose of 0.2 mg/kg compound. Blood samples taken before compound administration and on days 3 and 6 after compound administration were processed and analyzed by flow cytometry. Cells were first stained for CD25, CD3, NK1.1, CD4, CD8 and Zombie aqua fixable viability dye (Biolegend). After fixation and permeabilization (eBioscience) intracellular staining was performed for Ki67 and FoxP3. All compounds showed only slight increase in Ki67 expression in Treg cells on day 3, with levels decreasing on day 6 after compound administration. Proliferation (Ki67 expression) of CD8 T cells and NK cells in the blood of animals treated with bispecific compounds in the IgG format (FQQ111 and ONG682) or IgG-N-ter scFv (YIM345) format was comparable to QTY065, reaching maximal levels of Ki67+ cells (>75%) on day 6 after administration. Compounds with smaller molecular weight (Mw) and lacking the Fc domain, as HRL470 and VNP090, showed Ki67+ levels of 22-25% in CD8 T cells and 33-40% in NK cells. The bispecific format with C-terminal fusion of anti-hIL-2/IL-2 scFv on the anti-PD-1 IgG (CUM013) showed reduced potency compared to the other Fc-domain bearing compounds, with 47% Ki67+ NK cell and 25% Ki67+ CD8T cells, suggesting surprisingly, the IL-2 signaling was less potent when the cytokine was in a C-terminal linkage to a targeting antibody domain (FIG. 5).

### Example 7: In Vivo Anti-Tumor Efficacy

The B16F10 melanoma model, an aggressive, and immune checkpoint inhibitor (CPi) resistant tumor model, was selected to investigate additional protection conferred by non-blocking, PD-1-targeted moieties compared to anti-IL-2/IL-2 fusion proteins lacking PD-1 targeting. Immune cell proliferation and immunophenotyping of B16F10 subcutaneous tumors was performed in humanized PD-1 mice so that both PD-1 targeting and IL-2 activity could be assessed (C57BL/6N-Pdcd1tm1(huPDCD1-ICP11; Geno). Transgenic hPD-1 mice were injected s.c. in the right flank with 1×10⁶ B16F10 cells. When tumors reached an average size of 70 to 100 mm³, compounds were administered i.v. at a dose of 0.2 mg/kg. Tumor volumes were measured daily with a caliper and calculated with the formula (length×width×width)/2. Tumor volume differences in % compared to vehicle at study end at day 6 after treatment onset were calculated. Tumor infiltrating cells were analyzed by flow cytometry as described above, using the antibodies shown in Table 21.

TABLE 21
Antibodies used for intracellular (IC) and extracellular staining
(surface) for immunophenotyping of B16F10 tumors.

| | | | | | Catalog |
|---|---|---|---|---|---|
| | Antigen | Label | Clone | Supplier | No |
| Surface | CD45 | BV480 | 30-F11 | BD Biosciences | 566095 |
| | CD11b | BV605 | M1/70 | BD Biosciences | 563015 |
| | F4/80 | BV605 | T45-2342 | BD Biosciences | 743281 |
| | CD3 | BV650 | 145-2C11 | BD Biosciences | 564378 |
| | CD4 | PE-CF594 | RM4-5 | BD Biosciences | 562285 |
| | CD8 | R718 | H35-17.2 | BD Biosciences | 752290 |
| | CD25 | BB515 | PC61 | BD Biosciences | 564424 |
| | CD19 | SparkBlue | 6D5 | Biolegend | 115566 |
| | hPD1 | PE | A17188B | Biolegend | 621607 |
| | CX3CR1 | BV785 | SA011F11 | Biolegend | 149029 |
| | NK1.1 | BV711 | PK136 | BD Biosciences | 740663 |
| | CD49b | BB700 | HMα2 | BD Biosciences | 742140 |
| IC | FoxP3 | BV421 | MF23 | BD Biosciences | 562996 |
| | TCF1 | AF647 | C63D9 | Cell Signaling | 6709 |
| | Ki67 | PE-Cy7 | RMT3-23 | Biolegend | 119716 |

While administration of the untargeted anti-IL-2/IL-2 fusion protein QTY065 mainly led to increased intratumoral NK cells in the tumor of treated mice (8.2 fold compared to vehicle), all PD-1 targeted compounds drove a marked increase of CD8 T cells in B16F10 tumors. The smaller format HRL470 with double scFv fusion has the smallest effect (2.7 fold increase compared to vehicle), while the IgG and IgG-scFv fusion lead to CD8 T cell increase 4.6-8.1 fold compared to vehicle. Notably, the effect was enhanced within the target cell population, CD8+PD-1+ cells (Table 22). The C-terminal scFv compound CUM013, which did not show high potency in previous assays, showed comparable effects to the other bispecific formats on tumor infiltrating lymphocytes (TILs). Nonetheless, CUM013 had smaller effect on tumor growth inhibition compared to the other bispecifics (Table 23). Based on the results obtained, heterotetrameric IgG (CrossMab format) and IgG-N′terminal-scFv-IL-2-fusion format bispecific molecules were particularly effective in vivo, and the formats of VNP090 and CUM013 were not pursued further and shelved as potential backup.

TABLE 22
TILs from transgenic hPD-1 mice treated with untargeted or PD-1
targeted IL-2 antibody fusion protein as fold increase cells/gram
tumor over vehicle. Tumors analyzed on day 6 (n = 4).

| | Fold over vehicle cells/gram tumor | | | |
|---|---|---|---|---|
| Compound ID | Treg cells | NK cells | CD8 T cells | CD8 PD-1+ |
| QTY065 | 0.82 | 8.21 | 1.73 | 0.38 |
| HRL470 | 1.81 | 0.88 | 2.75 | 3.23 |
| VNP090 | 1.51 | 1.84 | 5.26 | 5.20 |
| FQQ111 | 1.25 | 1.12 | 4.62 | 5.52 |
| YMI345 | 0.97 | 1.78 | 5.32 | 6.36 |
| CUM013 | 0.83 | 0.80 | 8.14 | 9.83 |

TABLE 23
Tumor volume difference in % compared to mice treated
with vehicle on day 6, n = 4 mice per group.

| Compound | Tumor growth inhibition compared |
|---|---|
| ID | to vehicle (day 6) |
| QTY065 | −11.1% |
| HRL470 | 39.8% |
| VNP090 | 4.1% |
| FQQ111 | 46.6% |
| YMI345 | 33.0% |
| CUM013 | 8.7% |

In an additional study, tumor growth in hPD-1 transgenic mice was monitored after two administrations of bispecific compounds in comparison to vehicle, a non-blocking PD-1 antibody lacking IL-2 binding (the antibody XVT458 as human IgG1, with Fc silencing), or the untargeted bivalent antibody-IL-2 fusion QTY065. Day 0 corresponds to study start, when tumors reached 70-100 mm³. On day 0, and 3, 1.25 nmoles/kg of each compound were administered i.v. tumor growth was monitored and tumor volume difference was compared to mice treated with vehicle at end of study (Table 24). On day 5 after treatment, tumors were processed and TILs were analyzed by flow cytometry with intracellular staining as shown in Table 21. The inventors determined the fold increase compared to vehicle of cells/gram tumor of Treg cells, NK cells, CD8 T cells and CD8 T− cells subfamilies including stem-like pre-exhausted CD8 T cells (CD8+PD−1+ TCF1+), better effector exhausted T cells (CD8+ TCF1−CX3CR1+) and terminally exhausted T cells (CD8+ TCF1−CX3CR1−). The control antibody XVT458 induced only a minimal increase of the tumor cell infiltrate. The bispecific compounds induced in particular increase of CD8+PD-1+ T cells and derived subfamilies, with the heterotetrameric IgG CrossMab and IgG-scFv N-terminal format being the most efficacious compounds, with regards to target cell targeting, and anti-tumor potency (Tables 24 and 25).

TABLE 24
Tumor volume difference in % compared to
mice treated with vehicle on d5 (n = 5-6).

| | Tumor growth |
|---|---|
| Compound ID | inhibition % |
| QTY065 | 34.6% |
| HRL470 | 41.0% |
| FQQ111 | 70.5% |
| ONG682 | 59.3% |
| YMI345 | 54.5% |
| XVT458 | 17.4% |

TABLE 25
TILs from transgenic hPD-1 mice treated with untargeted or PD-1 targeted
IL-2 antibody fusion protein as fold increase cells/gram tumor over
vehicle. Tumors analyzed on day 5 post initiation of treatment.

| | Fold over vehicle cells/gram tumor | | | | | | |
|---|---|---|---|---|---|---|---|
| | | | | | | | CD8 + PD-1 + |
| Compound | Treg | NK | CD8 T | CD8 | CD8 + PD-1+ | CD8 + PD-1+ | TCF1− |
| ID | cells | cells | cells | PD-1+ | stem-like | better effector | exhausted |
| QTY065 | 1.7 | 63.0 | 4.9 | 3.5 | 13.1 | 3.4 | 5.3 |
| HRL470 | 2.2 | 3.6 | 3.1 | 3.0 | 3.9 | 2.8 | 4.5 |
| FQQ111 | 5.3 | 15.4 | 30.7 | 31.9 | 33.1 | 21.7 | 35.2 |
| ONG682 | 5.1 | 9.4 | 28.4 | 30.1 | 20.6 | 20.7 | 32.8 |
| YMI345 | 4.1 | 14.6 | 22.1 | 48.4 | 39.0 | 30.3 | 43.3 |
| XVT458 | 2.4 | 2.6 | 3.5 | 3.6 | 1.7 | 3.1 | 4.0 |

### Example 8: Increased In Vitro Potency of PD-1 Targeted Anti-hIL-2/IL-2 Fusion Proteins

Select bispecific formats were then combined with well-performing non-blocking anti-hPD-1 antibodies (Table 26). Antibodies targeting irrelevant antigens MDC982 and KVC110 were used as untargeted control antibodies. The anti-hIL-2/IL-2 arm from QTY065 has a light chain from the kappa subfamily, and can be combined with a second arm being from the lambda subfamily to form a heterotetrameric compound (a bispecific antibody where each antigen binding domain has a different specificity), without need for genetic engineering strategies such as CrossMab to ensure correct light chain pairing. Bispecifics in the heterotetrameric IgG kappa/lambda (FIG. 4F) format were designed combining the IL-2-anti-IL-2 fusion portion of QTY65 with two representative non-blocking, high affinity PD-1 antibodies 2A08Ap1 and 21A08Ap2 (both having a lambda light chain). Constructs encoding the sequences disclosed in Table 26 were transfected into Expi293 cells and produced and purified as described above. Knob in hole mutations were Y407T on HC1 and T366Y on HC2, and Fc silencing mutations L234A, L235A, P329A were incorporated.

TABLE 26
Combinations of QTY065 VH, VL(IL-2), and a second anti-hPD1 (or control)
antibody heterodimer in 3 different bispecific antibody formats.

| Anti-PD-1 | Bispecific antibody formats combined to QTY065 (kappa) | | |
|---|---|---|---|
| Antibody | (SEQ ID NO of polypeptide components) | | |
| (light chain) | IgG CrossMab | IgG-scFv N-terminal | IgG kappa/lambda |
| 21A08A p1 | YPW986 | LTJ498 | XWY176 |
| (lambda) | (088, 089, 090, 052) | (094, 095, 096) | (094, 095, 100, 052) |
| 21A08A p2 | PXU588 | JLI141 | GQM289 |
| (lambda) | (092, 093, 090, 052) | (097, 098, 099) | (097, 098, 100, 052) |
| XVT458-z2-m3 | RIB426 | TMU471 | — |
| (kappa) | (101, 102, 090, 052) | (105, 106, 107) | |
| XVT458-z2-m6 | QAB373 | MDS446 | — |
| (kappa) | (103, 104, 090, 052) | (108, 109, 110) | |
| Control Antibody | | | |
| (light chain) | IgG CrossMab | IgG-scFv N-terminal | IgG kappa/lambda |
| KVC110 | TSQ225 | LNX431 | — |
| (kappa) | | | |
| MDC982 | — | — | UVW948 |
| (lambda) | | | |

The bispecific compounds were tested for potency in inducing STAT5 phosphorylation in PD-1+ Jurkat cells, expressing the IL-2R CD122-CD132. Jurkat-PD1+CD122+ cells were activated with a serial dilution of bispecific compounds for 15 minutes at 37° C. and fixed by adding an equal volume of Cytofix buffer (BD Biosciences) for 10 minutes at 37° C. For the staining of intracellular antigens, cells were permeabilized with ice cold Perm buffer Ill (BD Biosciences) for 15 minutes on ice. Phosphorylated STAT5 was stained using an anti-pSTAT5 pY694 antibody (clone 47/Stat5, BD Biosciences). Flow cytometry was performed as previously described. EC50 values were calculated using the formula Y=Bottom+(X\hat{ }Hillslope)*(Top−Bottom)/(X\hat{ }HillSlope+EC50\hat{ }HillSlope) in Graphpad Prism V9.3.1 ([Agonist] vs. response −Variable slope (four parameters)). The PD-1 targeted bispecific compounds, except YPW986 and PXU588, showed increased potency and lower EC50 values compared to the bivalent QTY065 and the untargeted compound TSQ225 (Table 27). The binding ELISA assay suggested YPW986 and PXU588 (i.e. heterotetrameric IgG antibodies in a CrossMab bispecific format, with one arm derived from QTY065 and one either from 2108Ap1 or 21A08Ap2) lost their binding affinity to PD-1, in contrast to the heterotetrameric kappa/lambda IgG formats using the same non-blocking anti-PD-1 clones XWY176 and GQM289, which retained their ability to stimulate the dimeric IL-2 receptor. In general, PD-1 anchoring to the cells increased the signaling potency of the IL-2 on the bispecific compounds. The heterotetrameric Crossmab bispecific IgG FQQ111 with non-affinity matured anti-PD-1 arm (XVT458) induced a lower increase in potency by targeting to PD-1, indicating that higher affinity binding is desirable.

TABLE 27
EC50 values for STAT5 phosphorylation on Jurkat-
PD-1 + CD122 + cells.

| | EC50 value for pSTAT5 |
|---|---|
| Compound | induction (nM) |
| MDS446 | 0.06 |
| TMU471 | 0.06 |
| QAB373 | 0.07 |
| RIB426 | 0.12 |
| JLI141 | 0.20 |
| GQM289 | 0.22 |
| LTJ498 | 0.24 |
| XWY176 | 0.27 |
| FQQ111 | 0.62 |
| QTY065 | 1.07 |
| TSQ225 | 5.16 |
| PXU588 | 5.37 |
| YPW986 | 43.9 |

A further functional effect of a fusion protein according to the invention, is decrease of cell surface PD-1 upon binding to the IL-2R. Signaling through the IL-2R leads to internalization of the dimeric or trimeric receptor complex (Robb RJ. Et al. J Exp. Med. (1987) doi: 10.1084/jem.165.4.1201). Reduction of cell surface hPD-1 and CD122 by immunoconjugates of the present invention was tested by incubating the bispecifics at 3 different concentrations with stimulated PBMCs (i.e., PD-1+) for 16 hours. As controls, the bivalent anti-hIL-2/IL-2 compound QTY065 and the untargeted IL-2 compound as IgG CrossMab were used. Buffy coats or whole blood from healthy volunteers were received from the Blutspendezentrum SRK beider Basel or Aarau in compliance with the Swiss Human Research Act (HRA; May 2021) and other applicable ethics regulations by the Swiss ethics committee. PBMCs were isolated by density gradient centrifugation using Ficoll Plaque Plus (GE Healthcare). Frozen PBMCs were defrosted activated for 3 days with plate coated anti-CD3 (clone OKT3, BioLegend) and soluble anti CD28 (clone CD28.2, BioLegend). After activation PBMCs were incubated with bispecific compounds as indicated, overnight at 37° C. After incubation, cells were fixed immediately by adding an equal volume of Cytofix buffer (BD Biosciences) for 10 minutes at 37° C. Subsequently, surface markers were stained using antibodies listed in Table 28. Cells were acquired by flow cytometry as described above.

TABLE 28
Antibodies used for extracellular staining
for cell surface expression experiments.

| | | | | Catalog |
|---|---|---|---|---|
| Antigen | Label | Clone | Supplier | No |
| CD4 | PE-CF594 | SK3 | BD Biosciences | 566914 |
| CD8 | BV605 | SK1 | BD Biosciences | 564116 |
| CD122 | PE Cy7 | CF1 | BeckmanCoulter | A53365 |
| PD-1 | BB700 | EH12.1 | BD Biosciences | 746185 |
| CD3 | BV786 | SK7 | BD Biosciences | 563800 |
| CD132 | APC | TUGh4 | Biolegend | 338608 |
| CD25 | BV421 | 2A3 | BD Biosciences | 564033 |
| CD4 | PE-CF594 | SK3 | BD Biosciences | 566914 |

The difference in MFI of detected surface CD122 and PD-1 was calculated as % decrease compared to PBMC that were incubated only in medium. The control compounds induce decrease surface CD122, but leave PD-1 levels unchanged. For the PD-1 targeting compounds, both CD122 and PD-1 levels decreased. This may indicate that binding to the IL-2R is required for reduction of cell surface PD-1 (Table 29).

TABLE 29
Percentage decrease of CD122 and PD-1 MFI on CD8 T cells after
incubation with control compounds or bispecific compounds compared
to CD8 T cells incubated with medium alone (n = 2 donors).

| | % MFI decrease | | | | | |
|---|---|---|---|---|---|---|
| | CD122 | | | PD-1 | | |
| Compound | 25 nM | 5 nM | 1 nM | 25 nM | 5 nM | 1 nM |
| QTY065 | 76.6 | 67.0 | 41.6 | — | — | — |
| control | | | | | | |
| TSQ225 | 78.0 | 61.7 | 40.7 | — | — | — |
| control | | | | | | |
| LTJ498 | 59.1 | 36.2 | 7.8 | 46.4 | 46.7 | 41.8 |
| XWY176 | 62.9 | 40.1 | 9.4 | 52.4 | 52.7 | 42.0 |
| JLI141 | 60.7 | 35.3 | 5.7 | 38.6 | 45.0 | 38.0 |
| GQM289 | 57.4 | 39.9 | 9.7 | 46.4 | 44.8 | 25.7 |
| TMU471 | 59.8 | 43.4 | 24.7 | 34.6 | 46.4 | 45.0 |
| RIB426 | 56.9 | 41.3 | 15.4 | 43.3 | 46.7 | 33.2 |
| MDS446 | 76.5 | 59.5 | 32.2 | 29.1 | 37.3 | 31.1 |
| QAB373 | 63.3 | 41.6 | 24.4 | 33.0 | 30.6 | 27.3 |

### Example 9: Improved PD-1 Binders: In Vivo Ani-Tumor Efficacy and s.c. Tumor Immunophenotyping

The improved bispecific compounds of Table 26 were tested on hPD-1 transgenic mice bearing s.c. B16F10 tumors. Transgenic hPD-1 mice (C57BL/6N-Pdcd1tm1(huPDCD1-ICP11)Geno) were injected s.c. in the right flank with 1×10⁶ B16F10 cells. When tumors reached an average size of 70 to 100 mm³ (day 0), compounds were administered iv. at 1.25 nmoles/kg. A second administration was given on day 3. Mice were sacrificed on day 5 after study start and tumors were excised. Tumors were processed through a GentleMACS Octo Dissociator (Milteny) and cells were stained with NIR live dead stain (ThermoFisher, LI10119). Cells were incubated in Fc Block (TruStain FCX™, Biolegend, 101320) 10 min before adding the surface stain (see Table 21). Cells were fixed and permeabilized with FoxP3 Staining Buffer Set (ebioscience, #00-5523-00) before intracellular staining as per Table 21. All PD-1 targeted anti-IL-2/IL-2 bispecific antibodies markedly increase the intratumoral CD8 T cell numbers, in particular PD-1+ stem-like T cells and the derivatives compared to untargeted compounds or vehicle (Table 30). CD8 T cells Treg ratio was increased in the tumors of mice treated with bispecific compounds compared to vehicle or untargeted bispecifics.

TABLE 30
TILs from transgenic hPD-1 mice treated with untargeted or PD-1 targeted IL-2
antibody fusion protein as fold increase cells/gram tumor over vehicle. Last
column reports CD8 to Treg cells ratios. Tumors analyzed on day 5.

| | | | | | | | | CD8/Treg |
|---|---|---|---|---|---|---|---|---|
| | Fold over vehicle cells/gram tumor | | | | | | | ratio of |
| | | | | | CD8 + PD-1+ | CD8 + PD-1+ | CD8 + PD- | cells/ |
| | Treg | NK | CD8 T | CD8 | stem- | better | 1 + TCF1− | gram |
| Compound | cells | cells | cells | PD-1+ | like | effector | exhausted | tumor |
| Vehicle | — | — | — | — | — | — | — | 9.3 |
| RIB426 | 8.5 | 15.8 | 25.6 | 28.1 | 22.8 | 25.5 | 40.6 | 27.9 |
| TMU471 | 20.5 | 37.0 | 107.1 | 122.7 | 121.5 | 112.7 | 156.3 | 48.4 |
| QAB373 | 13.7 | 28.0 | 79.5 | 90.2 | 72.3 | 84.0 | 122.9 | 53.6 |
| MDS446 | 10.1 | 34.6 | 105.1 | 121.2 | 75.6 | 116.0 | 167.6 | 96.2 |
| XWY176 | 6.7 | 18.1 | 41.4 | 47.7 | 38.8 | 45.7 | 59.7 | 57.2 |
| LTJ498 | 12.4 | 29.0 | 69.1 | 81.0 | 51.1 | 78.1 | 110.5 | 51.6 |
| GQM289 | 8.0 | 18.8 | 27.1 | 29.6 | 24.4 | 26.6 | 42.6 | 31.4 |
| JLI141 | 2.7 | 8.2 | 29.5 | 35.6 | 16.8 | 33.1 | 56.2 | 100.4 |
| TSQ225 | 2.2 | 12.5 | 3.3 | 1.2 | 1.4 | 1.1 | 1.3 | 14.0 |
| LNX431 | 2.8 | 18.5 | 3.6 | 1.7 | 2.0 | 1.6 | 1.7 | 12.1 |

In an efficacy study, transgenic hPD-1 mice (C57BL/6N-Pdcd1tm1(huPDCD1-ICP11)Geno) were injected s.c. in the right flank with 1×10⁶ B16F10 cells. When tumors reached an average size of 70 to 100 mm³ (day 0), compounds were administered i.v. at 1.25 nmoles/kg. Additional administrations were given on day 3 and 7. Tumors were measured daily and volume was calculated with the formula (length×width×width)/2. Tumor volume difference between mice treated with vehicle or the respective compound n reported as % decrease compared to vehicle. All PD-1 targeted IL-2 fusion protein compounds effectively reduced tumor volume (Table 31).

TABLE 31
Tumor volume difference in % to vehicle on day 7.

| | % TGI vs |
|---|---|
| Compound | vehicle |
| Vehicle | — |
| TMU471 | 59 |
| QAB373 | 58 |
| MDS446 | 80 |
| XWY176 | 69 |
| LTJ498 | 62 |
| GQM289 | 64 |
| JLI141 | 80 |
| RIB426 | 65 |

### Example 10: Stress Study on Anti-PD-1, Anti-IL-2/IL-2 Bispecific Antibodies

Heterotetrameric bispecific antibodies XWY176, TMU471, QAB373 and MDS446 were produced with improved knob-in-hole mutations (S354C, T366W/S354C, T366S, L368A, Y407V) and Fc silencing (Table 32).

TABLE 32
Bispecific compounds with optimized knob-in
hole mutations and Fc silencing.

| Bispecific antibody name | |
|---|---|
| combined to QTY065 | Sequence ID |
| NZA596 | 111, 095, 112, 052 |
| KTX917 | 113, 106, 114 |
| CIT348 | 115, 109, 116 |
| BGY642 | 117, 104, 112, 052 |

The four bispecific antibodies from Table 32 were exposed to thermal, pH, oxidation, freeze-thaw stress conditions. After that the compounds were tested on SEC-HPLC (Table 33), iCEIF (Table 34) and functional ELISA (Table 35). Posttranslational modifications were analyzed by Mass spectrometry. The bispecific antibodies XWY176, TMU471, QAB373 and MDS446 were exposed to different stress conditions: i) incubation at 40° C. for 1 or 2 weeks, ii) 3 or 4 freeze-thaw cycles, iii) in 0.1% (v/v) H₂O₂ for 4 h or 24h, iv) low pH 3.5 for 24 or 48h, v) high pH 9.0 for 24 or 48h. To assess protein changes after the stress conditions the compounds were tested by SEC-HPLC, iCIEF, LC-MS. The functionality of the bispecific antibody was assessed with a sandwich ELISA that relies on its binding to the target antigen (hPD-1) and the integrity of the fused IL-2 via a secondary antibody (anti-IL-2 clone 5344). 60 nM of hPD-1 (ECD-His, produced in house) was coated on a Maxisorp plate (Nunc) overnight at 4° C. and blocked with 5% BSA in PBS. Bispecific antibodies were added in a serial dilution in assay buffer and detected with biotinylated-5344 and Streptavidin HRP (BD Pharmingen, 554066). Absorption end signal after adding TMB was read with a plate reader (Spectramax ID3) at 450 nm. EC50 values were determined by blotting Absorbance against concentration (Graphpad Prism, sigmoidal curve fit, 4 PL, X-axis is log). Change of EC50 values for the unstressed sample (TO) was compared to the stressed samples.

All constructs showed high potential for further development based on the criteria assessed in this Example. SEC-HPLC and iCIEF showed that the conditions inducing most protein changes were under thermal stress and high pH. The construct that showed less increase in high Mw (HMW) species by SEC-HPLC was NZA596. The same construct showed also less changes in charged species by iCIEF. The ELISA functionality assessment showed that all proteins retain at least 40% functionality after exposure to the different stress conditions. Constructs NZA596 and CIT348 retained >60% functionality at all conditions tested. PTM analysis showed methionine oxidation for all 4 molecules under thermal stress and oxidation conditions, among which NZA596 performed slightly better.

### Example 11: In Vitro Immunogenicity of Anti-PD-1, Anti-IL-2/IL-2 Bispecific Antibodies

In vitro immunogenicity of NZA596, CIT348, BGY642, KTX917 was tested by MHC-associated peptide proteomics (MAPPS), performed at Lonza. Monocyte-derived dendritic cells (DCs) from 10 different healthy donors (with variations in HLA-DRB alleles) were incubated with the protein of interest at 50 ug/ml and matured with LPS for 24 hours. After maturation the DC were lysed and the membrane fraction containing the HLA:peptide complexes was solubilized and incubated with Protein A mag 66epharose beads (Cytiva) coated with anti-HLA-DR antibody (Lonza) at 4° C. overnight. The following morning the beads were washed in TBS and the peptides eluted from the HLA-DR complex with 0.1% TFA. Finally, the peptides were purified by passing through a 10 kDa MWCO spin column and analyzed by MS. Peptide analysis revealed “immunogenic hotspots”, which correspond to HLA-presented peptides in 2 or more donors, that are not in the natural framework of an antibody. Table 36 summarizes the immunogenic hotspots identified per compound and the position of the peptide in the protein. The two non-blocking anti-PD-1 binding domains tested yielded similar hotspot profiles. All formats had acceptably low immunogenicity, but those with fewer mutated regions, and peptide linkers, most notably the kappa/lambda format, had the most favorable immunogenicity profile.

TABLE 33
SEC-HPLC of bispecific compounds a timepoint 0 (T0) or after different stress conditions:
40° C. 1 Week or 2 Weeks, Freeze-Thaw cycles, in oxidative conditions (0.1% H2O2)
4 hours or 24 hours, at low (pH 3.5) and high (pH 9.0) pH for 4 hours or 24 hours.

| | SEC-HPLC (Monomer %/HMW %/LMW %) | | | |
|---|---|---|---|---|
| Condition | NZA596 | CIT348 | BGY642 | KTX917 |
| T0 | 98.8/1.2/0.0 | 98.2/1.6/0.2 | 99.0/1.0/0.0 | 98.5/1.1/0.4 |
| 40° C.-1 W | 98.5/0.9/0.6 | 96.1(↓2.1)/2.9/0.9 | 98.3(↓0.7)/0.8/0.9 | 94.6(↓3.9)/4.0/1.3 |
| 40° C.-2 W | 98.3(↓0.5)/0.7/1.0 | 91.9(↓6.3)/6.7/1.4 | 96.8(↓2.2)/2.0/1.2 | 90.4(↓8.1)/7.9/1.7 |
| Freeze/thaw-3 cycles | 98.7/1.3/0.0 | 98.1/1.7/0.2 | 99.0/1.0/0.0 | 98.4/1.1/0.5 |
| Freeze/thaw-5 cycles | 98.8/1.2/0.0 | 98.2/1.6/0.2 | 99.0/1.0/0.0 | 98.4/1.1/0.5 |
| 0.1% (v/v) H₂O₂-4 h | 98.9/1.1/0.0 | 98.6/1.3/0.2 | 99.4/0.6/0.0 | 98.5/1.1/0.4 |
| 0.1% (v/v) H₂O₂-24 h | 98.9/1.1/0.0 | 98.1/1.4/0.5 | 99.6/0.4/0.0 | 98.9/0.7/0.4 |
| pH 3.5-4 h | 99.2/0.8/0.0 | 97.8/1.8/0.4 | 99.0/1.0/0.0 | 98.8/0.7/0.4 |
| pH 3.5-48 h | 99.1/0.9/0.0 | 97.6(↓0.6)/1.8/0.7 | 99.1/0.9/0.0 | 98.2/1.1/0.6 |
| pH 9.0-4 h | 98.1(↓0.7)/1.9/0.0 | 96.5(↓1.7)/3.3/0.2 | 98.4(↓0.6)/1.6/0.0 | 98.0(↓0.5)/1.1/1.0 |
| pH 9.0-48 h | 98.2(↓0.6)/1.8/0.1 | 96.1(↓2.1)/3.5/0.4 | 98.3(↓0.7)/1.5/0.1 | 96.1(↓2.4)/3.4/0.5 |

HMW: High molecular weight; LMW: Low molecular weight.

TABLE 34
Capillary isoelectric focusing of bispecific compounds a timepoint 0 (T0) or after different
stress conditions: 40° C. 1 Week or 2 Weeks, Freeze-Thaw cycles, in oxidative conditions
(0.1% H2O2) 4 hours or 24 hours, at low (pH 3.5) and high (pH 9.0) pH for 4 hours or 24 hours.

| | iCIEF (Main peak %/Acidic peaks %/Basic peaks %) | | | |
|---|---|---|---|---|
| Condition | NZA596 | CIT348 | BGY642 | KTX917 |
| T0 | 58.5/40.1/1.5 | 58.3/33.6/8.2 | 60.9/30.9/8.2 | 53.0/42.7/4.3 |
| 40° C.-1 W | 50.0(↓8.5)/47.9/2.1 | 35.4(↓22.9)/59.3/5.3 | 36.3(↓24.6)/54.2/9.5 | 23.8(↓29.2)/74.0/2.2 |
| 40° C.-2 W | 38.5(↓20.0)/58.8/2.6 | 21.6(↓36.7)/74.6/3.8 | 28.0(↓32.9)/65.1/6.9 | 15.1(↓37.9)/84.9/0.0 |
| Freeze/thaw-3 cycles | 57.8/40.5/1.7 | 57.3/34.4/8.3 | 61.7/30.0/8.3 | 51.8/43.8/4.4 |
| Freeze/thaw-5 cycles | 58.2/40.4/1.4 | 58.1/34.4/7.4 | 60.4/30.4/9.1 | 53.3/41.3/5.4 |
| 0.1% (v/v) H₂O₂-4 h | 57.2/41.7/1.1 | 56.4/35.6/8.0 | 62.8/28.7/8.5 | 51.6/43.9/4.4 |
| 0.1% (v/v) H₂O₂-24 h | 55.3(↓3.2)/43.2/1.5 | 50.6(↓7.7)/41.7/7.8 | 61.9/29.6/8.5 | 45.3(↓7.7)/49.4/5.3 |
| pH 3.5-4 h | 58.7/39.3/2.0 | 57.6/34.7/7.7 | 61.0/31.3/7.7 | 52.3/43.0/4.7 |
| pH 3.5-48 h | 59.3/38.7/2.0 | 57.2/34.9/8.0 | 61.4/30.4/8.2 | 52.7/42.2/5.2 |
| pH 9.0-4 h | 56.7/41.71.6 | 55.2(↓3.1)/36.9/7.9 | 60.3/31.7/8.1 | 50.1(↓2.9)/45.2/4.7 |
| pH 9.0-48 h | 52.7(↓5.8)/45.9/1.4 | 50.2(↓8.1)/42.4/7.5 | 58.0(↓2.9)/33.9/8.1 | 46.3(↓6.7)/49.3/4.3 |

TABLE 35
Functional ELISA of bispecific compounds a timepoint 0 (T0)
or after different stress conditions: Full functionality of
T0 set as 100% and EC50 values of stressed samples in % of
T0 EC50. 40° C. 2 Weeks, in oxidative conditions (0.1%
H2O2) 24 hours, at low (pH 3.5) and high (pH 9.0) pH 24 hours.

| | Sample | | | |
|---|---|---|---|---|
| Condition | NZA596 | CIT348 | BGY642 | KTX917 |
| T0 | 100 | 100 | 100 | 100 |
| 40° C.-2 W | 63 | 67 | 44 | 49 |
| 0.1% (v/v) H₂O₂-24 h | 84 | 85 | 86 | 92 |
| pH 3.5-48 h | 97 | 96 | 94 | 100 |
| pH 9.0-48 h | 87 | 94 | 91 | 103 |

TABLE 36
Immunogenic hotspots of the bispecific compounds.

| | Immunogenic hotspots | |
|---|---|---|
| | # hotspots | Locations |
| BGY642 | 2 | CrossMab substitution |
| | | CDR-L anti-PD-1 |
| NZA596 | 1 | CDR-L anti-PD-1 |
| CIT438 | 2 | CDR-L anti-PD-1 |
| | | scFv linkers |
| KTX917 | 4 | CDR-L anti-PD-1 |
| | | CDR-H anti-PD-1 hole |
| | | CDR-H anti-PD-1 knob |
| | | scFv linkers |

### Example 12: Mass Spectrometry Showing Correct Light Chain Pairing

To test correct light chain pairing of the purified bispecific constructs in the IgG CrossMab or IgG kappa/lambda formats digestion by IgdE enzyme was performed and the Fab fragments were analyzed by Mass spectrometry. One single peak was visible for each of the Fabs being assayed, corresponding to the theoretical molecular weight of correctly assembled light chain and heavy chain (FIG. 6). No incorrect light chain pairing was detected.

### Example 13: Cis-Signaling in Combination with PD-1 Blocking Antibodies

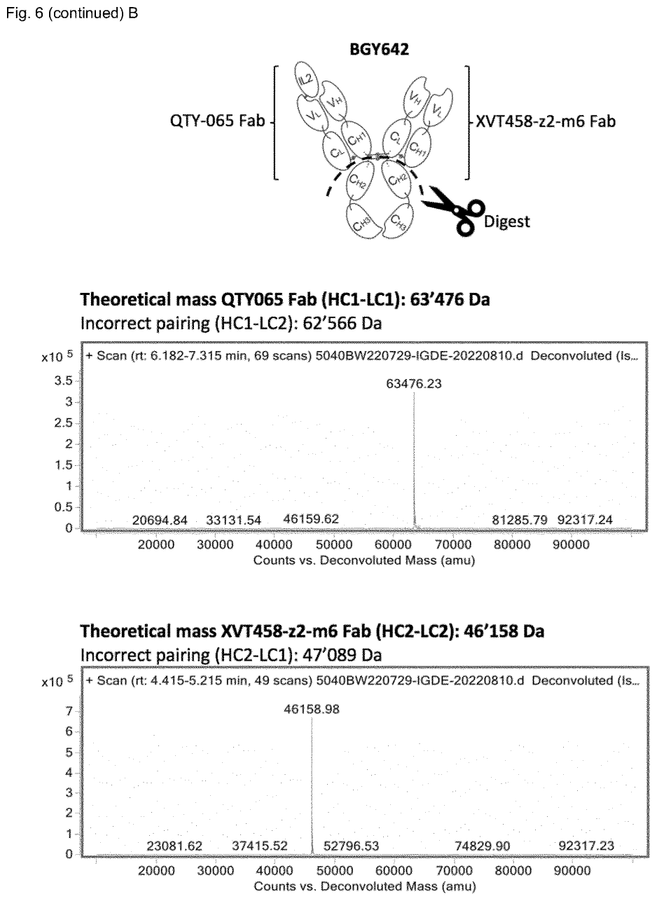

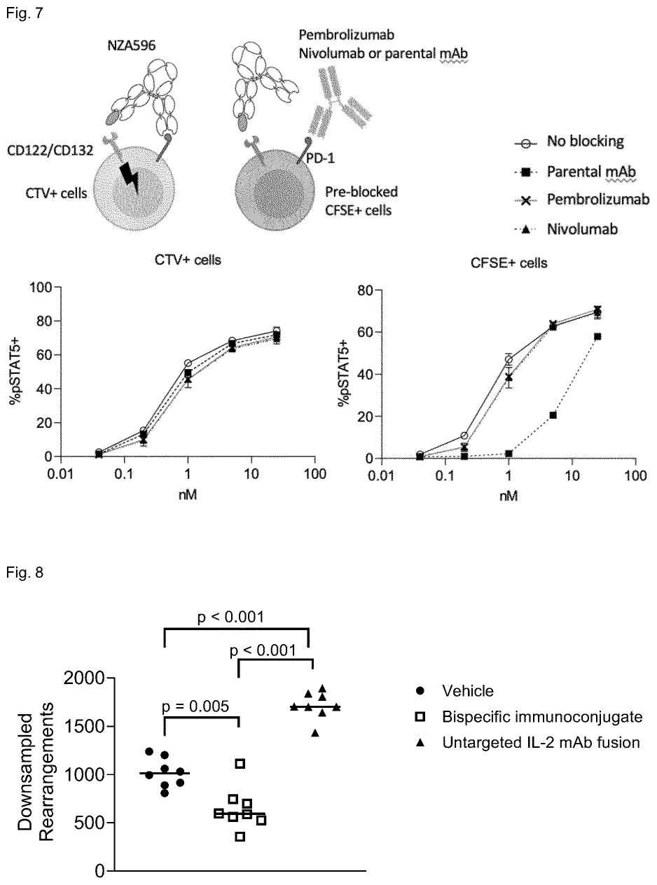

An assay was established to assess whether an exemplary bispecific construct as disclosed herein delivers the IL-2 to 00122-00132 on the PD-1 expressing cell bound by its anti-PD-1 arm (cis-signaling) or signals to a neighboring cell (trans). Jurkat-PD-1+ CD122+ cells were either labeled with CFSE (Invitrogen, 034557) or CTV (Invitrogen 034554). CFSE labeled cells were then exposed to 700 nm of the non-competing parent antibody to PD-1, Pembrolizumab or Nivolumab, to block PD-1 epitopes for 30 minutes at room temperature. After two washes, both CTV and CFSE labeled cells were mixed at a 1:1 ratio and activated with the bispecific compound NZA596 (1 nM) for 15 minutes at 37° C. After the stimulation period, cells were fixed immediately by adding an equal volume of Cytofix buffer (BD Biosciences 554655) for 10 minutes at 37° C. For the staining of intracellular antigens, cells were permeabilized with ice cold Perm buffer Ill (BD Biosciences 558050) for 15 minutes on ice. Phosphorylated STAT5 was stained using an anti-pSTAT5 pY694 antibody (clone 47/Stat5, BD Biosciences). Induction of STAT5 phosphorylation through the immunoconjugate in these cells was analyzed. The potency of the immunoconjugate is markedly reduced on cells that were pre-incubated with the parent antibody. This interference is not observed on cells were that were pre-exposed to Pembrolizumab or Nivolumab (Table 37, FIG. 7). Furthermore, the potency of the immunoconjugate on cells that had not been exposed to any of the PD-1 binding antibodies before was the same for all samples (co-incubated with CFSE+ pre-blocked cells), indicating that the immunoconjugate signals in a cis manner, on the same cells where PD-1 binding occurs (Table 37, FIG. 7).

TABLE 37
% of pSTAT5 compared to non-blocked cells
after stimulation with 1 nM NZA596:

% pSTAT5

| PD1 binder | CFSE labeled cells | CTV labeled cells |
|---|---|---|
| Non blocked | 100.00 | 100.00 |
| parent AB | 4.81 | 89.80 |
| Pembrolizumab | 82.59 | 82.56 |
| Nivolumab | 81.39 | 82.44 |

### Example 14: Anti-Tumor Efficacy of Bispecific Immunoconjugate in Combination with Pembrolizumab and Nivolumab in Two Mouse Models of Cancer

The efficacy of an exemplary bispecific immunoconjugate (NZA596) disclosed in this application was tested as monotherapy or in combination with commercially available PD-1 blocking agents Pembrolizumab and Nivolumab. hPD-1 transgenic C57BL/6 mice were injected with s.c. B16F10 melanoma cells or MC38 colorectal tumor cells. When tumors reached an average size of 70-100 mm³ mice were randomized and treatment was started as described in Table 38. Mice were administered on days 0 (day of randomization) and day 3. Pembrolizumab or Nivolumab were administered also on day 7. Tumor growth inhibition was calculated on day 13 for MC38 tumor bearing mice and on day 14 for B16F10 tumor bearing mice compared to tumor volumes of mice treated with Vehicle. In both tumor models the bispecific immunoconjugate combined to either of the checkpoint inhibitors induced marked tumor growth retardation even at low doses, compared to vehicle. Combination treatments led to stronger tumor growth inhibition compared to any of the single agent treatments.

TABLE 38
Treatment schedules and doses for in vivo efficacy study in hPD-1 transgenic mice
bearing B16F10 or MC38 tumors. Tumor growth inhibition (TGI) was calculated as
percentage volume decrease compared to mean volume of Vehicle treated group.

| | Compound 1 | Compound 2 | TGI | | | | |
|---|---|---|---|---|---|---|---|
| | Days 0, 3 | Days 0, 3, 7 | d 9 | TGI | | TGI | |
| | intravenous | Intraperitoneal | MC38 | d 11 MC38 | | d 14 B16F10 | |
| Treatment | (i.v.) | (i.p.) | model | model | | model | |
| Vehicle | PBS | PBS | — | — | (n 5) | — | (n 5) |
| Pembrolizumab | PBS | Pembrolizumab | 41.6% | 30.0% | (n 7) | 35.1% | (n 9) |
| | | 10 mg/kg | | | | | |
| Nivolumab | PBS | Nivolumab | 41.5% | 38.6% | (n 6) | 36.7% | (n 8) |
| | | 10 mg/kg | | | | | |
| NZA596 dose 1* | NZA596 | PBS | 35.4% | 11.7% | (n 8) | 61.2% | (n 10) |
| NZA596 dose 2** | NZA596 | PBS | 57.2% | 48.7% | (n 9) | 51.2% | (n 9) |
| NZA596 dose 1* + | NZA596 | Pembrolizumab | 65.7% | 63.1% | (n 9) | 69.8% | (n 10) |
| Pembrolizumab | | 10 mg/kg | | | | | |
| NZA596 dose 2* + | NZA596 | Pembrolizumab | 74.2% | 78.3% | (n 8) | 75.9% | (n 10) |
| Pembrolizumab | | 10 mg/kg | | | | | |
| NZA596 dose 1* + | NZA596 | Nivolumab | 76.7% | 76.8% | (n 9) | 78.0% | (n 10) |
| Nivolumab | | 10 mg/kg | | | | | |
| NZA596 dose 2* + | NZA596 | Nivolumab | 81.8% | 80.8% | (n 9) | 83.0% | (n 10) |

| | |
|---|---|
| Nivolumab | 10 mg/kg |

*Dose 1: MC38 model: 0.1 mg/kg, B16F10 model: 0.2 mg/kg
**Dose 2: MC38 model: 0.2 mg/kg, B16F10 model: 0.4 mg/kg.
(n) = number of animals.

### Example 15: Potency in pSTAT5 Signaling in Presence of Various Existing PD-1 Blocking Agents

STAT5 phosphorylation of Jurkat PD-1+CD122+ cells was analyzed by flow cytometry after 15 minutes incubation with a dilution series of an exemplary bispecific immunoconjugate (NZA596). Before stimulation cells were either incubated for 30-60 minutes with growth medium only, existing PD-1 blocking agents or the parental anti-PD-1 antibody 21A08Ap1, all at saturating concentration (200 nM). NZA506 was added to the cells without washing the anti-PD-1 IgGs. pSTAT5+ cells were detected by flow cytometry. The EC50 of the bispecific immunoconjugate NZA596 in stimulating Jurkat PD-1+ CD122+ cells was calculated plotting the concentration against the % pSTAT5+ cells to be 0.85 nM. This value was markedly increased when the parental anti-PD-1 antibody 21A08Ap1 was used for pre-blocking PD-1 on the cells. When cells were pre-incubated with Pembrolizumab, Nivolumab, Cemiplimab, Dostarlimab, Zeluvalimab, Tislelizumab, Ezabenlimab, Toripalimab, or Cetrelimab the potency in pSTAT5 induction of NZA596 remained unchanged, indicating that blocking of the PD-L1-PD-1 axes with these antibody is possible without altering the activity of the bispecific immunoconjugates describes in this patent (Table 39). The difference in binding epitopes of PD-1 was also demonstrated by SPR showing simultaneous binding of NZA596 and commercially available PD-1 blocking antibodies on immobilized recombinant PD-1 (data not shown).

TABLE 39
List of anti-PD-1 antibodies pre-incubated with Jurkat
PD-1 + CD122 + cells before stimulation with
the bispecific immunoconjugate NZA596. Potency (EC50)
value in nM and fold reduction based on pre-incubation with medium.

| | | fold reduction |
|---|---|---|
| Pre-incubation with | EC50 (nM)* | in NZA596 Potency |
| Medium | 0.85 | N/A |
| 21A08Ap1 | 15.55 | 18.19 |
| Pembrolizumab | 1.02 | 1.20 |
| Nivolumab | 1.10 | 1.28 |
| Cemiplimab | 1.67 | 1.95 |
| Dostarlimab | 0.96 | 1.12 |
| Zeluvalimab | 0.88 | 1.03 |
| Tislelizumab | 1.47 | 1.72 |
| Ezabenlimab | 1.71 | 2.00 |
| Toripalimab | 0.65 | 0.77 |
| Cetrelimab | 0.76 | 0.89 |

### Example 16: Bispecific Immunoconjugate Induces the Amplification of Tumor Specific Subsets of T Cells in the Tumor Microenvironment

In the tumor context, PD-1 marks T cells that have been chronically exposed to tumor antigens and therefore marks the subset of T cells with the relevant anti-tumor specificities. To gain a better understanding of the repertoire of T cells that was expanded by the bispecific immunoconjugate of the present invention, T cell receptor beta (TCRβ) chain sequencing of TILs from B16F10 tumor bearing hPD-1 mice was performed, and the TCR repertoire diversity assessed. Transgenic hPD-1 mice were injected s.c. with B16F10 cells. On study day 0, when tumors reached a size of 50-100 mm³, the mice were randomized and treated i.v. with 0.2 mg/kg bispecific immunoconjugate (targeted to PD-1), 0.2 mg/kg non-PD-1 targeted immunoconjugate (QTY065), or vehicle, followed by a second dose on day 3. The mice were sacrificed on day 5 and genomic DNA was extracted from the tumors for sequencing of the TCR3 repertoire. Mice treated with the PD-1 targeted bispecific immunoconjugate showed a decreased TCR repertoire as a result of the proliferation of a focused, likely tumor-antigen specific, subset of T cell clones derived from the PD-1+ cells present before treatment (FIG. 8). In contrast the non-PD-1 targeted immunoconjugate induced an increased TCR repertoire richness compared to vehicle, indicating that this untargeted IL-2Rβ/γ agonist expanded a more diverse set of T cell clones, that likely included many non-tumor relevant specificities.

To quantify the effects of the bispecific immunoconjugate on CD8+ T cells with defined tumor-neoantigen specificity, the dynamics of the antigen specific T cell response among TILs from MC38 or B16F10-OVA bearing mice were analyzed by flow cytometry, with MHC dextramers. Wild-type C57BL/6 mice bearing s.c. B16F10-OVA or MC38 tumors were treated with 0.2 mg/kg of a mouse surrogate of the bispecific immunoconjugate BGY642 (named DSQ964, targeted to mouse PD-1 by the antigen-binding domain of clone RMP1-30, which does not impede PD-1 binding to inhibitory ligands), untargeted immunoconjugate (QTY065) or vehicle on day 0 after randomization (tumor volume 50-100 mm³) and on day 3. On day 5 TILs were isolated and analyzed by flow cytometry using dextramers that specifically stain CD8+ T cells which recognize the ovalbumin neoantigen expressed by the B16F10-OVA tumor cell line and the p15E retroviral antigen expressed by MC38 tumor cell line. In both tumor models, the tumor neoantigen specific T cells were increased in mice treated with DSQ964 compared to vehicle or untargeted IL-2/anti-IL-2 fusion protein (Table 40). In addition, all tumor-antigen specific CD8 T cells were found to be PD-1+. Together with the TCR sequencing data, these experiments confirm that the PD-1 targeted bispecific immunoconjugate expands a specific subset of CD8+ T cells that recognize tumor cells in mouse models of cancer.

TABLE 40
Fold increase of intratumoral Dextramer + CD8 + PD-1 +
T cells in DSQ964 or QTY065 treated mice over mice treated with
vehicle in B16F10-OVA (n = 6 in vehicle, n = 8 in DQS964,
QTY065 groups) or MC38 tumors (n = 9).

| | Fold over vehicle | Fold over vehicle |
|---|---|---|
| | CD8 + PD-1 + OVA- | CD8 + PD-1 + OVA- |
| | Dextramer + T cells in | Dextramer + T cells |
| | B16F10-OVA model | in B16F10-OVA model |
| DSQ964 | 10.8 | 4.7 |
| QTY065 | 0.1 | 2.7 |

| Sequences: | |
|---|---|
| SEQ ID NO 001: Antibody A HC (artificial): VH (CDRs)- CH_{1,2,3} | |
| EVQLVQSGAEVKKPGESLKISCKGSGYAFTNYLIEWVRQMPGKGLEWMGVINPGSGGTNYNE | |
| KFKGQVTISADKSISTAYLQWSSLKASDTAMYYCARWRGEGYYAYFDVWGQGTTVTVSSAST | |
| KGPSVFPLAPSSKSTSGGTAALGCLVKDYFPEPVTVSWNSGALTSGVHTFPAVLQSSGLYSL | |
| SSVVTVPSSSLGTQTYICNVNHKPSNTKVDKRVEPKSCDKTHTCPPCPAPELLGGPSVFLFP | |
| PKPKDTLMISRTPEVTCVVVDVSHEDPEVKFNWYVDGVEVHNAKTKPREEQYASTYRVVSVL | |
| TVLHQDWLNGKEYKCKVSNKALPAPIEKTISKAKGQPREPQVYTLPPSREEMTKNQVSLTCL | |
| VKGFYPSDIAVEWESNGQPENNYKTTPPVLDSDGSFFLYSKLTVDKSRWQQGNVFSCSVMHE | |
| ALHNHYTQKSLSLSPGK | |
| SEQ ID NO 002: Antibody A LC (artificial): VL (CDRs) - CL | |
| AIRLTQSPSSFSASTGDRVTITCKASQSVDYQGDSYMNWYQQKPGKAPKLLIYAASNLESGV | |
| PSRFSGSGSGTDFTLTISSLQSEDFATYYCQQSNEDPYTFGGGTKVEIKRTVAAPSVFIFPP | |
| SDEQLKSGTASVVCLLNNFYPREAKVQWKVDNALQSGNSQESVTEQDSKDSTYSLSSTLTLS | |
| KADYEKHKVYACEVTHQGLSSPVTKSFNRGEC | |
| SEQ ID NO 003: Antibody B HC (artificial): VH (CDRs) - CH_{1,2,3} | |
| QVQLVESGGGVVQPGRSLRLSCAASGYTFSSYLIEWVRQAPGKGLEWVAVINPGSGGTNYAD | |
| SVKGRFTISADKSKNTAYLQMNSLRAEDTAVYYCARWRGDGYYAYFDVWGQGTTVTVSSAST | |
| KGPSVFPLAPSSKSTSGGTAALGCLVKDYFPEPVTVSWNSGALTSGVHTFPAVLQSSGLYSL | |
| SSVVTVPSSSLGTQTYICNVNHKPSNTKVDKRVEPKSCDKTHTCPPCPAPELLGGPSVFLFP | |
| PKPKDTLMISRTPEVTCVVVDVSHEDPEVKFNWYVDGVEVHNAKTKPREEQYNSTYRVVSVL | |
| TVLHQDWLNGKEYKCKVSNKALPAPIEKTISKAKGQPREPQVYTLPPSREEMTKNQVSLTCL | |
| VKGFYPSDIAVEWESNGQPENNYKTTPPVLDSDGSFFLYSKLTVDKSRWQQGNVFSCSVMHE | |
| ALHNHYTQKSLSLSPGK | |
| SEQ ID NO 004: Antibody B LC (artificial): VL (CDRs) - CL | |
| EIVLTQSPATLSVSPGERATLSCRASQSVSYDGDSYMNWYQQKPGQAPRLLIYAASNLASGI | |
| PARFSGSGSGTEFTLTISSLQSEDAAVYYCQQSNEDPYTFGGGTKVEIKRTVAAPSVFIFPP | |
| SDEQLKSGTASVVCLLNNFYPREAKVQWKVDNALQSGNSQESVTEQDSKDSTYSLSSTLTLS | |
| KADYEKHKVYACEVTHQGLSSPVTKSFNRGEC | |
| SEQ ID NO 005: IL-2 (human) | |
| MYRMQLLSCIALSLALVTNSAPTSSSTKKTQLQLEHLLLDLQMILNGINNYKNPKLTRMLTF | |
| KFYMPKKATELKHLQCLEEELKPLEEVLNLAQSKNFHLRPRDLISNINVIVLELKGSETTFM | |
| CEYADETATIVEFLNRWITFCQSIISTLT | |
| SEQ ID NO 006: Proleukin (artificial) | |
| MAPTSSSTKKTQLQLEHLLLDLQMILNGINNYKNPKLTRMLTFKFYMPKKATELKHLQCLEE | |
| ELKPLEEVLNLAQSKNFHLRPRDLISNINVIVLELKGSETTFMCEYADETATIVEFLNRWIT | |
| FSQSIISTLT | |
| SEQ ID NO 007: no alpha IL-2 mutein (artificial) | |
| APTSSSTKKTQLQLEHLLLDLQMILNGINNYKNPKLTAMLTAKFAMPKKATELKHLQCLEEA | |
| LKPLEEVLNLAQSKNFHLRPRDLISNINVIVLELKGSETTFMCEYADETATIVEFLNRWITF | |
| CQSIISTLT | |
| SEQ ID NO 008: W02012/107417A1 IL2 mutein (artificial) | |
| APTSSSTKKTQLQLEHLLLDLQMILNGINNYKNPKLTRMLTAKFAMPKKATELKHLQCLEEE | |
| LKPLEEVLNGAQSKNFHLRPRDLISNINVIVLELKGSETTFMCEYADETATIVEFLNRWITF | |
| CQSIISTLT | |
| SEQ ID NO 009: IL-2 superkine (artificial) | |
| APTSSSTKKTQLQLEHLLLDLQMILNGINNYKNPKLTRMLTFKFYMPKKATELKHLQCLEEE | |
| LKPLEEVLNLAQSKNFHFDPRDVVSNINVFVLELKGSETTFMCEYADETATIVEFLNRWITF | |
| CQSIISTLT | |
| SEQ ID NO 010: Circularly permuted Proleukin variant 1 | |
| (artificial) | |
| NFHLRPRDLISNINVIVLELKGSETTFMCEYADETATIVEFLNRWITFSQSIISTLTPTSSS | |
| TKKTQLQLEHLLLDLQMILNGINNYKNPKLTRMLTFKFYMPKKATELKHLQCLEEELKPLEE | |
| VLNLAQSK | |
| SEQ ID NO 011: Circularly permuted Proleukin variant 2 | |
| (artificial) | |
| FHLRPRDLISNINVIVLELKGSETTFMCEYADETATIVEFLNRWITFSQSIISTLTPTSSST | |
| KKTQLQLEHLLLDLQMILNGINNYKNPKLTRMLTFKFYMPKKATELKHLQCLEEELKPLEE | |
| VLNLAQSK | |
| SEQ ID NO 012: Circularly permuted Proleukin variant 3 | |
| (artificial) | |
| FYMPKKATELKHLQCLEEELKPLEEVLNLAQSKNFHLRPRDLISNINVIVLELKGSETTFMC | |
| EYADETATIVEFLNRWITFSQSIISTLTPTSSSTKKTQLQLEHLLLDLQMILNGINNYKNPK | |
| LTRMLTFK | |
| SEQ ID NO 013: Circularly permuted Proleukin variant 4 | |
| (artificial) | |
| ATIVEFLNRWITFCQSIISTLTPTSSSTKKTQLQLEHLLLDLQMILNGINNYKNPKLTRMLT | |
| FKFYMPKKATELKHLQCLEEELKPLEEVLNLAQSKNFHLRPRDLISNINVIVLELKGSETTE | |
| MCEYADET | |
| SEQ ID NO 014: Peptide linker (artificial): G | |
| SEQ ID NO 015: Peptide linker (artificial): GG | |
| SEQ ID NO 016: Peptide linker (artificial): GGG | |
| SEQ ID NO 017: Peptide linker (artificial): GGGG | |
| SEQ ID NO 018: Peptide linker (artificial): G₄S | |
| SEQ ID NO 019: Peptide linker (artificial): G₄SG | |
| SEQ ID NO 020: Peptide linker (artificial): G₄SGG | |
| SEQ ID NO 021: Peptide linker (artificial): (G₄S)₂GGG | |
| SEQ ID NO 022: Peptide linker (artificial): (G₄S)₂GGGG | |
| SEQ ID NO 023: Peptide linker (artificial): (G₄S)₃GGGG | |
| SEQ ID NO 024: Peptide linker (artificial): (G₄S)₄ | |
| SEQ ID NO 025: EAD406 LC (artificial): VL(CDR) IL-2CP Linker | |
| AIRLTQSPSSFSASTGDRVTITCKASQSVDYNFHLRPRDLISNINVIVLELKGSETTFMCEY | |
| ADETATIVEFLNRWITFSQSIISTLTPTSSSTKKTQLQLEHLLLDLQMILNGINNYKNPKLT | |
| RMLTFKFYMPKKATELKHLQCLEEELKPLEEVLNLAQSKGDSYMNWYQQKPGKAPKLLIYAA | |
| SNLESGVPSRFSGSGSGTDFTLTISSLQSEDFATYYCQQSNEDPYTFGGGTKVEIK | |
| SEQ ID NO 026: XFQ227 LC (artificial): VL(CDR) IL-2CP Linker | |
| AIRLTQSPSSFSASTGDRVTITCKASQSVDYGGNFHLRPRDLISNINVIVLELKGSETTEMC | |
| EYADETATIVEFLNRWITFSQSIISTLTPTSSSTKKTQLQLEHLLLDLQMILNGINNYKNPK | |
| LTRMLTFKFYMPKKATELKHLQCLEEELKPLEEVLNLAQSKGGGDSYMNWYQQKPGKAPKL | |
| LIYAASNLESGVPSRFSGSGSGTDFTLTISSLQSEDFATYYCQQSNEDPYTFGGGTKVEIK | |
| SEQ ID NO 027: QTY065 LC (artificial): VL(CDR) IL-2CP Linker | |
| AIRLTQSPSSFSASTGDRVTITCKASQSVDYGGGNFHLRPRDLISNINVIVLELKGSETTFM | |
| CEYADETATIVEFLNRWITFSQSIISTLTPTSSSTKKTQLQLEHLLLDLQMILNGINNYKNP | |
| KLTRMLTFKFYMPKKATELKHLQCLEEELKPLEEVLNLAQSKGGGGDSYMNWYQQKPGKAPK | |
| LLIYAASNLESGVPSRFSGSGSGTDFTLTISSLQSEDFATYYCQQSNEDPYTFGGGTKVEIK | |
| SEQ ID NO 028: FJC828 LC (artificial): VL(CDR) IL-2CP Linker | |
| AIRLTQSPSSFSASTGDRVTITCKASQSVDYGGGGNFHLRPRDLISNINVIVLELKGSETTE | |
| MCEYADETATIVEFLNRWITFSQSIISTLTPTSSSTKKTQLQLEHLLLDLQMILNGINNYKN | |
| PKLTRMLTFKFYMPKKATELKHLQCLEEELKPLEEVLNLAQSKGGGGSDSYMNWYQQKPGKA | |
| PKLLIYAASNLESGVPSRFSGSGSGTDFTLTISSLQSEDFATYYCQQSNEDPYTFGGGTKVE | |
| IK | |
| SEQ ID NO 029: PGQ345 LC (artificial): VL(CDR) IL-2CP Linker | |
| constant | |
| AIRLTQSPSSFSASTGDRVTITCKASQSVDYGGGGSGNFHLRPRDLISNINVIVLELKGSET | |
| TFMCEYADETATIVEFLNRWITFSQSIISTLTPTSSSTKKTQLQLEHLLLDLQMILNGINNY | |
| KNPKLTRMLTFKFYMPKKATELKHLQCLEEELKPLEEVLNLAQSKGGGGSGGDSYMNWYQQK | |
| PGKAPKLLIYAASNLESGVPSRFSGSGSGTDFTLTISSLQSEDFATYYCQQSNEDPYTFGGG | |
| TKVEIK | |
| SEQ ID NO 030: DRV470 LC (artificial): VL(CDR) IL-2CP Linker | |
| AIRLTQSPSSFSASTGDRVTITCKASQSVDYGGGGSGGGGSGGGNFHLRPRDLISNINVIVL | |
| ELKGSETTFMCEYADETATIVEFLNRWITFSQSIISTLTPTSSSTKKTQLQLEHLLLDLQMI | |
| LNGINNYKNPKLTRMLTFKFYMPKKATELKHLQCLEEELKPLEEVLNLAQSKGGGGSGGGGS | |
| GGGGDSYMNWYQQKPGKAPKLLIYAASNLESGVPSRFSGSGSGTDFTLTISSLQSEDFATYY | |
| CQQSNEDPYTFGGGTKVEIK | |
| SEQ ID NO 031: XUB802 LC (artificial): VL(CDR) IL-2CP Linker | |
| AIRLTQSPSSFSASTGDRVTITCKASQSVDYGGGGSGGGGSGGGGSGGGGNFHLRPRDLISN | |
| INVIVLELKGSETTFMCEYADETATIVEFLNRWITFSQSIISTLTPTSSSTKKTQLQLEHLL | |
| LDLQMILNGINNYKNPKLTRMLTFKFYMPKKATELKHLQCLEEELKPLEEVLNLAQSKGGGG | |
| SGGGGSGGGGSGGGGSDSYMNWYQQKPGKAPKLLIYAASNLESGVPSRFSGSGSGTDFTLTI | |
| SSLQSEDFATYYCQQSNEDPYTFGGGTKVEIK | |
| SEQ ID NO 032: EPK959 LC (artificial): VL(CDR) IL-2CP Linker | |
| EIVLTQSPATLSVSPGERATLSCRASQSVSYNFHLRPRDLISNINVIVLELKGSETTEMCEY | |
| ADETATIVEFLNRWITFSQSIISTLTPTSSSTKKTQLQLEHLLLDLQMILNGINNYKNPKLT | |
| RMLTFKFYMPKKATELKHLQCLEEELKPLEEVLNLAQSKGDSYMNWYQQKPGQAPRLLIYAA | |
| SNLASGIPARFSGSGSGTEFTLTISSLQSEDAAVYYCQQSNEDPYTFGGGTKVEIK | |
| SEQ ID NO 033: GYG794 LC (artificial): VL(CDR) IL-2CP Linker | |
| EIVLTQSPATLSVSPGERATLSCRASQSVSYGGNFHLRPRDLISNINVIVLELKGSETTFMC | |
| EYADETATIVEFLNRWITFSQSIISTLTPTSSSTKKTQLQLEHLLLDLQMILNGINNYKNPK | |
| LTRMLTFKFYMPKKATELKHLQCLEEELKPLEEVLNLAQSKGGGDSYMNWYQQKPGQAPRLL | |
| IYAASNLASGIPARFSGSGSGTEFTLTISSLQSEDAAVYYCQQSNEDPYTFGGGTKVEIK | |
| SEQ ID NO 034: DRQ069 LC (artificial): VL(CDR) IL-2CP Linker | |
| EIVLTQSPATLSVSPGERATLSCRASQSVSYGGGNFHLRPRDLISNINVIVLELKGSETTFM | |
| CEYADETATIVEFLNRWITFSQSIISTLTPTSSSTKKTQLQLEHLLLDLQMILNGINNYKNP | |
| KLTRMLTFKFYMPKKATELKHLQCLEEELKPLEEVLNLAQSKGGGGDSYMNWYQQKPGQAPR | |
| LLIYAASNLASGIPARFSGSGSGTEFTLTISSLQSEDAAVYYCQQSNEDPYTFGGGTKVEIK | |
| SEQ ID NO 035: ECV200 LC (artificial): VL(CDR) IL-2CP Linker | |
| EIVLTQSPATLSVSPGERATLSCRASQSVSYGGGGSGNFHLRPRDLISNINVIVLELKGSET | |
| TFMCEYADETATIVEFLNRWITFSQSIISTLTPTSSSTKKTQLQLEHLLLDLQMILNGINNY | |
| KNPKLTRMLTFKFYMPKKTELKHLQCLEEELKPLEEVLNLAQSKGGGGSGGDSYMNWYQQKA | |
| PGQAPRLLIYAASNLASGIPARFSGSGSGTEFTLTISSLQSEDAAVYYCQQSNEDPYTFGGG | |
| TKVEIK | |
| SEQ ID NO 036: BFC885 LC (artificial): VL(CDR) IL-2CP Linker | |
| EIVLTQSPATLSVSPGERATLSCRASQSVSYGGGGSGGGGSGGGNFHLRPRDLISNINVIVL | |
| ELKGSETTFMCEYADETATIVEFLNRWITFSQSIISTLTPTSSSTKKTQLQLEHLLLDLQMI | |
| LNGINNYKNPKLTRMLTFKFYMPKKATELKHLQCLEEELKPLEEVLNLAQSKGGGGSGGGGS | |
| GGGGDSYMNWYQQKPGQAPRLLIYAASNLASGIPARFSGSGSGTEFTLTISSLQSEDAAVYY | |
| CQQSNEDPYTFGGGTKVEIK | |
| SEQ ID NO 037: LPT269 LC (artificial): VL(CDR) IL-2CP Linker | |
| constant | |
| DIQMTQSPSSVSASVGDRVTITCSASQGISGNFHLRPRDLISNINVIVLELKGSETTEMCEY | |
| ADETATIVEFLNRWITFSQSIISTLTPTSSSTKKTQLQLEHLLLDLQMILNGINNYKNPKLT | |
| RMLTFKFYMPKKATELKHLQCLEEELKPLEEVLNLAQSKGDLNWYQQKPGKAPKLLIYHTSS | |
| LHSGVPSRFSGSGSGTDFTLTISSLQPEDFATYYCQYYSKDLLTFGGGTKLEIK | |
| SEQ ID NO 038: DXM339 LC (artificial): VL(CDR) IL-2CP Linker | |
| DIQMTQSPSSVSASVGDRVTITCSASQGISGGGNFHLRPRDLISNINVIVLELKGSETTFMC | |
| EYADETATIVEFLNRWITFSQSIISTLTPTSSSTKKTQLQLEHLLLDLQMILNGINNYKNPK | |
| LTRMLTFKFYMPKKATELKHLQCLEEELKPLEEVLNLAQSKGGGDLNWYQQKPGKAPKLLIY | |
| HTSSLHSGVPSRFSGSGSGTDFTLTISSLQPEDFATYYCQYYSKDLLTFGGGTKLEIK | |
| SEQ ID NO 039: FUE433 LC (artificial): VL(CDR) IL-2CP Linker | |
| DIQMTQSPSSVSASVGDRVTITCSASQGISGGGGNFHLRPRDLISNINVIVLELKGSETTEM | |
| CEYADETATIVEFLNRWITFSQSIISTLTPTSSSTKKTQLQLEHLLLDLQMILNGINNYKNP | |
| KLTRMLTFKFYMPKKATELKHLQCLEEELKPLEEVLNLAQSKGGGGDLNWYQQKPGKAPKLL | |
| IYHTSSLHSGVPSRFSGSGSGTDFTLTISSLQPEDFATYYCQYYSKDLLTFGGGTKLEIK | |
| SEQ ID NO 040: LQM346 LC (artificial): VL(CDR) IL-2CP Linker | |
| DIQMTQSPSSVSASVGDRVTITCSASQGISGGGGGSGNFHLRPRDLISNINVIVLELKGSET | |
| TFMCEYADETATIVEFLNRWITFSQSIISTLTPTSSSTKKTQLQLEHLLLDLQMILNGINNY | |
| KNPKLTRMLTFKFYMPKKATELKHLQCLEEELKPLEEVLNLAQSKGGGGSGGDLNWYQQKPG | |
| KAPKLLIYHTSSLHSGVPSRFSGSGSGTDFTLTISSLQPEDFATYYCQYYSKDLLTFGGGTK | |
| LEIK | |
| SEQ ID NO 041: GLK754 LC (artificial): VL(CDR) IL-2CP Linker | |
| DIQMTQSPSSVSASVGDRVTITCSASQGISGGGGGSGGGGSGGGNFHLRPRDLISNINVIVL | |
| ELKGSETTFMCEYADETATIVEFLNRWITFSQSIISTLTPTSSSTKKTQLQLEHLLLDLQMI | |
| LNGINNYKNPKLTRMLTFKFYMPKKATELKHLQCLEEELKPLEEVLNLAQSKGGGGSGGGGS | |
| GGGGDLNWYQQKPGKAPKLLIYHTSSLHSGVPSRFSGSGSGTDFTLTISSLQPEDFATYYCQ | |
| YYSKDLLTFGGGTKLEIK | |
| SEQ ID NO 042: Antibody A VL (CDRs) | |
| AIRLTQSPSSFSASTGDRVTITCKASQSVDYQGDSYMNWYQQKPGKAPKLLIYAASNLESGV | |
| PSRFSGSGSGTDFTLTISSLQSEDFATYYCQQSNEDPYTFGGGTKVEIK | |
| SEQ ID NO 043: Antibody A VH (CDRs) | |
| EVQLVQSGAEVKKPGESLKISCKGSGYAFTNYLIEWVRQMPGKGLEWMGVINPGSGGTNYNE | |
| KFKGQVTISADKSISTAYLQWSSLKASDTAMYYCARWRGEGYYAYFDVWGQGTTVTVSS | |
| SEQ ID NO 044: Antibody B VH (CDRs) | |
| QVQLVESGGGVVQPGRSLRLSCAASGYTFSSYLIEWVRQAPGKGLEWVAVINPGSGGTNYAD | |
| SVKGRFTISADKSKNTAYLQMNSLRAEDTAVYYCARWRGDGYYAYFDVWGQGTTVTVSS | |
| SEQ ID NO 045: Antibody C VH (CDRs) | |
| EVQLVQSGAEVKKPGESLKISCKGSGYSFTNFYIHWVRQAPGQRLEWMGSIYPNYGDTAYNQ | |
| KFKDRFVFSLDTSVSTAYLQISSLKAEDTAVYYCARGYSYAMDYWGQGTTVTVSS | |
| SEQ ID NO 046: VBE401 VH (artificial): CDR, IL-2CP, linkers | |
| EVQLVQSGAEVKKPGESLKISCKGSGYAFTNYLIEWVRQMPGKGLEWMGVINPGSGGTNYNE | |
| KFKGQVTISADKSISTAYLQWSSLKASDTAMYYCARWRGEGGGFHLRPRDLISNINVIVLEL | |
| KGSETTEMCEYADETATIVEFLNRWITFSQSIISTLTPTSSSTKKTQLQLEHLLLDLQMILN | |
| GINNYKNPKLTRMLTFKFYMPKKATELKHLQCLEEELKPLEEVLNLAQSKGGGGYYAYFDVW | |
| GQGTTVTVSS | |
| SEQ ID NO 047: LIZ707 VH (artificial): CDR, IL-2CP, linkers | |
| EVQLVQSGAEVKKPGESLKISCKGSGYAFTNYLIEWVRQMPGKGLEWMGVINPGGGFYMPKK | |
| ATELKHLQCLEEELKPLEEVLNLAQSKNFHLRPRDLISNINVIVLELKGSETTFMCEYADET | |
| ATIVEFLNRWITFCQSIISTLTPTSSSTKKTQLQLEHLLLDLQMILNGINNYKNPKLTRMLT | |
| FKGGGSGGTNYNEKFKGQVTISADKSISTAYLQWSSLKASDTAMYYCARWRGEGYYAYFDVW | |
| GQGTTVTVSS | |
| SEQ ID NO 048: Fusion protein IL-2 antigen binding domain HC | |
| constant region | |
| ASTKGPSVFPLAPSSKSTSGGTAALGCLVKDYFPEPVTVSWNSGALTSGVHTFPAVLQSSGL | |
| YSLSSVVTVPSSSLGTQTYICNVNHKPSNTKVDKRVEPKSCDKTHTCPPCPAPELLGGPSVF | |
| LFPPKPKDTLMISRTPEVTCVVVDVSHEDPEVKFNWYVDGVEVHNAKTKPREEQYASTYRVV | |
| SVLTVLHQDWLNGKEYKCKVSNKALPAPIEKTISKAKGQPREPQVYTLPPSREEMTKNQVSL | |
| TCLVKGFYPSDIAVEWESNGQPENNYKTTPPVLDSDGSFFLYSKLTVDKSRWQQGNVFSCSV | |
| MHEALHNHYTQKSLSLSPGK | |
| SEQ ID NO 049: Fusion protein IL-2 antigen binding domain LC | |
| constant region | |
| RTVAAPSVFIFPPSDEQLKSGTASVVCLLNNFYPREAKVQWKVDNALQSGNSQESVTEQDSK | |
| DSTYSLSSTLTLSKADYEKHKVYACEVTHQGLSSPVTKSENRGEC | |
| SEQ ID NO 050: Antibody B VL CDR | |
| EIVLTQSPATLSVSPGERATLSCRASQSVSYDGDSYMNWYQQKPGQAPRLLIYAASNLASGI | |
| PARFSGSGSGTEFTLTISSLQSEDAAVYYCQQSNEDPYTFGGGTKVEIK | |
| SEQ ID NO 051: QTY065 VH (CDR), constant | |
| EVQLVQSGAEVKKPGESLKISCKGSGYAFTNYLIEWVRQMPGKGLEWMGVINPGSGGTNYNE | |
| KFKGQVTISADKSISTAYLQWSSLKASDTAMYYCARWRGEGYYAYFDVWGQGTTVTVSSAST | |
| KGPSVFPLAPSSKSTSGGTAALGCLVKDYFPEPVTVSWNSGALTSGVHTFPAVLQSSGLYSL | |
| SSVVTVPSSSLGTQTYICNVNHKPSNTKVDKRVEPKSCDKTHTCPPCPAPELLGGPSVFLFP | |
| PKPKDTLMISRTPEVTCVVVDVSHEDPEVKFNWYVDGVEVHNAKTKPREEQYASTYRVVSVL | |
| TVLHQDWLNGKEYKCKVSNKALPAPIEKTISKAKGQPREPQVYTLPPSREEMTKNQVSLTCL | |
| VKGFYPSDIAVEWESNGQPENNYKTTPPVLDSDGSFFLYSKLTVDKSRWQQGNVFSCSVMHE | |
| ALHNHYTQKSLSLSPGK | |
| SEQ ID NO 052: QTY065 LC CDR, IL-2CP, linkers, constant | |
| AIRLTQSPSSFSASTGDRVTITCKASQSVDYGGGNFHLRPRDLISNINVIVLELKGSETTFM | |
| CEYADETATIVEFLNRWITFSQSIISTLTPTSSSTKKTQLQLEHLLLDLQMILNGINNYKNP | |
| KLTRMLTFKFYMPKKATELKHLQCLEEELKPLEEVLNLAQSKGGGGDSYMNWYQQKPGKAPK | |
| LLIYAASNLESGVPSRFSGSGSGTDFTLTISSLQSEDFATYYCQQSNEDPYTFGGGTKVEIK | |
| RTVAAPSVFIFPPSDEQLKSGTASVVCLLNNFYPREAKVQWKVDNALQSGNSQESVTEQDSK | |
| DSTYSLSSTLTLSKADYEKHKVYACEVTHQGLSSPVTKSENRGEC | |
| SEQ ID NO 053: Z0-XVT458 Heavy chain: VH (CDRs)-CH_{1,2,3} | |
| QVQLVQSGAEVKKPGASVKVSCKASGYTFTNFYIHWVRQAPGQGLEWMGSIYPNYGDTAYNQ | |
| KFKDRVTMTTDTSTSTAYMELRSLRSDDTAVYYCARGYSYAMDYWGQGTTVTVSSASTKGPS | |
| VFPLAPSSKSTSGGTAALGCLVKDYFPEPVTVSWNSGALTSGVHTFPAVLQSSGLYSLSSVV | |
| TVPSSSLGTQTYICNVNHKPSNTKVDKKVEPKSCDKTHTCPPCPAPELLGGPSVFLFPPKPK | |
| DTLMISRTPEVTCVVVDVSHEDPEVKFNWYVDGVEVHNAKTKPREEQYNSTYRVVSVLTVLH | |
| QDWLNGKEYKCKVSNKALPAPIEKTISKAKGQPREPQVYTLPPSRDELTKNQVSLTCLVKGF | |
| YPSDIAVEWESNGQPENNYKTTPPVLDSDGSFFLYSKLTVDKSRWQQGNVFSCSVMHEALHN | |
| HYTQKSLSLSPGK | |
| SEQ ID NO 054: Light chain: Z0-XVT458 Light chain VL (CDRs)-CL | |
| DIQMTQSPSSVSASVGDRVTITCSASQGISGDLNWYQQKPGKAPKLLIYHTSSLHSGVPSRF | |
| SGSGSGTDFTLTISSLQPEDFATYYCQYYSKDLLTFGGGTKLEIKRTVAAPSVFIFPPSDEQ | |
| LKSGTASVVCLLNNFYPREAKVQWKVDNALQSGNSQESVTEQDSKDSTYSLSSTLTLSKADY | |
| EKHKVYACEVTHQGLSSPVTKSENRGEC | |
| SEQ ID NO 055: z2-XVT458 VH (CDRs) | |
| QVQLVQSGAEVKKPGASVKVSCKASGYTFTNFYIHWVRQAPGQGLEWMGSIYPNYGDTAYNQ | |
| KFKDRVTMTVDTSTSTAYMELRSLRSDDTAVYYCARGYSYAMDYWGQGTTVTVSS | |
| SEQ ID NO 056: z2-XVT458 VL (CDRs) | |
| DIQMTQSPSSVSASVGDRVTITCSASQGISGDLNWYQQKPGKAPKLLIYHTSSLHSGVPSRF | |
| SGSGSGTDFTLTISSLQPEDFATYYCQYYSKDLLTFGGGTKLEIK | |
| SEQ ID NO 057: z2_XVT458m1 VH (CDRs) mutations in bold | |
| QVQLVQSGAEVKKPGASVKVSCKASGYTFTNFYIHWVRQAPGQGLEWMGSIYPNYGFTAYNQ | |
| KFKDRVTMTVDTSTSTAYMELRSLRSDDTAVYYCARGYSYAMDYWGQGTTVTVSS | |
| SEQ ID NO 058: z2_XVT458m1 VL (CDRs) mutations in bold | |
| DIQMTQSPSSVSASVGDRVTITCSASQGIWGDLNWYQQKPGKAPKLLIYHTSQLHSDVPSRF | |
| SGSGSGTDFTLTISSLQPEDFATYYCQGYSKDLLTFGGGTKLEIK | |
| SEQ ID NO 059: z2_XVT458m2 VH (CDRs) mutations in bold | |
| QVQLVQSGAEVKKPGASVKVSCKASGYTFTNFYIHWVRQAPGQGLEWMGRIYPNYGFTAYNQ | |
| KFKDRVTMTVDTSTSTAYMELRSLRSDDTAVYYCARGYSYAMDYWGQGTTVTVSS | |
| SEQ ID NO 060: z2_XVT458m2 VL (CDRs) mutations in bold | |
| DIQMTQSPSSVSASVGDRVTITCSASQGIYGDLNWYQQKPGKAPKLLIYHTSSLHSGVPSRF | |
| SGSGSGTDFTLTISSLQPEDFATYYCQAYSKDLLTFGGGTKLEIK | |
| SEQ ID NO 061: z2_XVT458m3 VH (CDRs) mutations in bold | |
| QVQLVQSGAEVKKPGASVKVSCKASGYTFTNFYIHWVRQAPGQGLEWMGRIYPNYGITAYNQ | |
| KFKDRVTMTVDTSTSTAYMELRSLRSDDTAVYYCARGYSYAMDYWGQGTTVTVSS | |
| SEQ ID NO 062: z2_XVT458m3 VL (CDRs) mutations in bold | |
| DIQMTQSPSSVSASVGDRVTITCSASQGISGDLNWYQQKPGKAPKLLIYHTSQRHSDVPSRF | |
| SGSGSGTDFTLTISSLQPEDFATYYCQGYSKDLLTFGGGTKLEIK | |
| SEQ ID NO 063: z2_XVT458m4 VH (CDRs) mutations in bold | |
| QVQLVQSGAEVKKPGASVKVSCKASGYTFTNFYIHWVRQAPGQGLEWMGRIYPNYGITAYNQ | |
| KFKDRVTMTVDTSTSTAYMELRSLRSDDTAVYYCARGYSYAMDYWGQGTTVTVSS | |
| SEQ ID NO 064: z2_XVT458m4 VL (CDRs) mutations in bold | |
| DIQMTQSPSSVSASVGDRVTITCSASQGISGDLNWYQQKPGKAPKLLIYHTSSRHSGVPSRF | |
| SGSGSGTDFTLTISSLQPEDFATYYCQGYSKDLLTFGGGTKLEIK | |
| SEQ ID NO 065: z2_XVT458m5 VH (CDRs) mutations in bold | |
| QVQLVQSGAEVKKPGASVKVSCKASGYTFTNFYIHWVRQAPGQGLEWMGRIYPNYGITAYNQ | |
| KFKDRVTMTVDTSTSTAYMELRSLRSDDTAVYYCARGYSYAMDYWGQGTTVTVSS | |
| SEQ ID NO 066: z2_XVT458m5 Light chain: VL (CDRs) mutations | |
| in bold | |
| DIQMTQSPSSVSASVGDRVTITCSASQGIYGDLNWYQQKPGKAPKLLIYHTSSRHSGVPSRF | |
| SGSGSGTDFTLTISSLQPEDFATYYCQAYSKDLLTFGGGTKLEIK | |
| SEQ ID NO 067: z2_XVT458m6 Heavy chain: VH (CDRs)-mutations | |
| in bold | |
| QVQLVQSGAEVKKPGASVKVSCKASGYTFTNFYIHWVRQAPGQGLEWMGSIYPNYGITAYNQ | |
| KFKDRVTMTVDTSTSTAYMELRSLRSDDTAVYYCARGYSYAMDYWGQGTTVTVSS | |
| SEQ ID NO 068: z2_XVT458m6 Light chain: VL (CDRs) mutations | |
| in bold | |
| DIQMTQSPSSVSASVGDRVTITCSASQGISGDLNWYQQKPGKAPKLLIYHTSQLHSDVPSRF | |
| SGSGSGTDFTLTISSLQPEDFATYYCQGYSKDLLTFGGGTKLEIK | |
| SEQ ID NO 069: ZJN296-0 VH (CDRs) | |
| QVQLVQSGAEVKKPGASVKVSCKASGYDFTSHWMHWVRQAPGQGLEWMGAIDPSDSYTNYNQ | |
| KFKGRVTMTTDTSTSTAYMELRSLRSDDTAVYYCARSPFDYWGQGTTVTVSS | |
| SEQ ID NO 070: ZJN296-0 VL (CDRs) | |
| DIQMTQSPSSLSASVGDRVTITCKASQDVSTAVAWYQQKPGKAPKLLIYSASYRYTGVPSRF | |
| SGSGSGTDFTFTISSLQPEDIATYYCQQHYSFPFTFGQGTKLEIK | |
| SEQ ID NO 071: 21A08 Heavy chain: VH (CDRs) | |
| EVQLLESGGGLVQPGGSLRLSCAASGFTFSINAMTWVRQAPGKGLEWVSTISGSGFSTYYAD | |
| SLKGRFTISRDNSKNTLYLQMNSLRAEDTAVYYCAKEVYGDYWGQGTLVTVSS | |
| SEQ ID NO 072: 21A08 Light chain: VL (CDRs)-CL (lamda) | |
| RSVLTQPPSASGTPGQRVSISCSGNSSNIGSNSVFWYQQLPGTAPKLLIYSNNQRPSGVPDR | |
| ESGSKSGTSASLAISGLRSEDEADYYCAAWDDSLSIWVFGGGTKLTVL | |
| SEQ ID NO 073: 22F13 Heavy chain: VH (CDRs) | |
| EVQLLESGGGLVQPGGSLRLSCAASGFTFSISAMTWVRQAPGKGLEWVSTISGSGGSTYYSD | |
| SVKGRFTISRDNSKNTLYLQINSLRADDTAVYYCAKEVYGDYWGQGTLVTVSS | |
| SEQ ID NO 074: 22F13 Light chain: VL (CDRs) | |
| QSILTQPPSASGTPGQRVTISCSGSSSNIGSNSVFWYQQLPGTAPKLLIFSNNQRPSGVPD | |
| RESGSKSGTSASLAISGLRSEDEADYCCAAWDDSLSGWIFGGGTKLTVL | |
| SEQ ID NO 075: 25120 Heavy chain: VH (CDRs) | |
| EVQLLESGGNLVQPGGSLRLSCAASGFTFSTYAMSWVRQAPGKGLEWVSTISGTGYSTYFAD | |
| SVKGRATISRDNSKNTLFLQMNSLRAEDTAVYYCAKEVYGDYWGQGTLVTVSS | |
| SEQ ID NO 076: 25120 Light chain: VL (CDRs) | |
| QSVLTQPPSASGTPGQRVTISCSGRSSNIGSNSIFWYQQLPGTAPKLLIYSNNQRPSGVPDR | |
| ESGSKSGTSASLAISGLRSEDETDYYCAAWDDSLSGWVFGGGTKLTVL | |
| SEQ ID NO 077: 20H02 Heavy chain: VH (CDRs) | |
| EVLLVESGGGLVQPGGSLRLSCAASGFTFSSYSMNWVRQAPGKGLEWVSFISSSSPTLYYAD | |
| SVKGRFTISRDNAKNSLYLQMNSLRDEDTAVYYCARQGLTPFDYWGQGTLVTVSS | |
| SEQ ID NO 078: 20H02 Light chain: VL (CDRs) | |
| EIVMTQSPATLSVFPGERATLSCRASQSVSSYLAWYQQKPGQAPRLLIYGASTRATGIPARV | |
| SGSGSGTEFILTISSLQSEDFAVYYCQQYNNWPYTFGQGTKLEIK | |
| SEQ ID NO 079: 39F23 Heavy chain: VH (CDRs) | |
| QVQLVESGGGVGQPGRSLRLSCAASGFTFSNYGMHWVRQAPGKGLEWVAVIWYDGSKKYYAD | |
| SVKGRFTISRDNSKNTLYLQMNSLRVEDTAVYYCVGNSGHWGQGTLVTVSS | |
| SEQ ID NO 080: 39F23 Light chain: VL (CDRs) | |
| DIVMTQTPLSLPVTPGEPASISCRSSQSLLDSDDGNTYLDWYLQKPGQSPQLLIYTLSYRAS | |
| GVPDRESGSGSGTDFTLKISRVEAEDVGVYYCMQCIEFPHTFGGGTKVEIK | |
| SEQ ID NO 081: 40B20 Heavy chain: VH (CDRs) | |
| EVQLVESGGGLVQPGGSLRLSCAASGFTFSSYSMNWVRQAPGKGLQWVSYITSSSNTMYYAD | |
| SVKGRETISRDNAKNSLYLQMNSLRDEDTAVYYCASIVGAPFDYWGQGTLVTVSS | |
| SEQ ID NO 082: 40B20 Light chain: VL (CDRs) | |
| EIVMTQSPATLSVSPGERATLSCRASQSVSSSLAWYQQKPGQAPRLLIYGASTRATGIPARF | |
| SGSGSGTEFTLTISSLQSEDFAAYYCQQYNNWPFTFGPGTKVDIK | |
| SEQ ID NO 083: 56H02 Heavy chain: VH (CDRs) | |
| QVQLVQSGAEVKKPGASVKVSCKASGYTFTSYFMHWVRQAPGQGLEWMGLINPDGGNTDYAQ | |
| KFQGRVTMTRDTSTSTVYMELSSLRSEDTAVYYCARDGNYYDSSGYYYPDAFDIWGQGTMVT | |
| VSS | |
| SEQ ID NO 084: 56H02 Light chain: VL (CDRs) | |
| SSELTQDPAVSVALGQTVRITCQGDSLRRFYASWYQQKPGQAPVIVIYGKDNRPSGTPDRFS | |
| GSTSGNTASLTITGAQAEDEADYYCNSRDSSGTHVVFGGGTKLTVL | |
| SEQ ID NO 085: 21A08Ap1, 21A08Ap2 VH (CDRs) | |
| EVQLLESGGGLVQPGGSLRLSCAASGFTFSINAMTWVRQAPGKGLEWVSTISGSGFSTYYAD | |
| SLKGRFTISRDNSKNTLYLQMNSLRAEDTAVYYCAKEVYGDYWGQGTLVTVSS | |
| SEQ ID NO 086: 21A08Ap1 VL (CDRs) | |
| RSVLTQPPSASGTPGQRVSISCSGASSNIGSQSVFWYQQLPGTAPKLLIYSNNQRPSGVPDR | |
| ESGSKSGTSASLAISGLRSEDEADYYCAAWDDSLSIWVFGGGTKLTVL | |
| SEQ ID NO 087: 21A08Ap2 VL (CDRs) | |
| RSVLTQPPSASGTPGQRVSISCSGASSNIGSSSVFWYQQLPGTAPKLLIYSNNQRPSGVPDR | |
| FSGSKSGTSASLAISGLRSEDEADYYCAAWDDSLSIWVFGGGTKLTVL | |
| SEQ ID NO 088: YPW986 21A08A p1 VH (CDR)-CLK-Hinge-CH2-CH3 | |
| EVQLLESGGGLVQPGGSLRLSCAASGFTFSINAMTWVRQAPGKGLEWVSTISGSGESTYYAD | |
| SLKGRFTISRDNSKNTLYLQMNSLRAEDTAVYYCAKEVYGDYWGQGTLVTVSSASVAAPSVF | |
| IFPPSDEQLKSGTASVVCLLNNFYPREAKVQWKVDNALQSGNSQESVTEQDSKDSTYSLSST | |
| LTLSKADYEKHKVYACEVTHQGLSSPVTKSFNRGECDKTHTCPPCPAPEAAGGPSVFLFPPK | |
| PKDTLMISRTPEVTCVVVDVSHEDPEVKFNWYVDGVEVHNAKTKPREEQYNSTYRVVSVLTV | |
| LHQDWLNGKEYKCKVSNKALAAPIEKTISKAKGQPREPQVYTLPPSREEMTKNQVSLTCLVK | |
| GFYPSDIAVEWESNGQPENNYKTTPPVLDSDGSFFLTSKLTVDKSRWQQGNVFSCSVMHEAL | |
| HNHYTQKSLSLSPGK | |
| SEQ ID NO 089: YPW986 21A08A p1 VL (CDR)-CH1 | |
| RSVLTQPPSASGTPGQRVSISCSGASSNIGSQSVFWYQQLPGTAPKLLIYSNNQRPSGVPDR | |
| ESGSKSGTSASLAISGLRSEDEADYYCAAWDDSLSIWVFGGGTKLTVLSSASTKGPSVFPLA | |
| PSSKSTSGGTAALGCLVKDYFPEPVTVSWNSGALTSGVHTFPAVLQSSGLYSLSSVVTVPSS | |
| SLGTQTYICNVNHKPSNTKVDKRVEPKSC | |
| SEQ ID NO 090: QTY065: VH (CDR) -constant (knob) | |
| EVQLVQSGAEVKKPGESLKISCKGSGYAFTNYLIEWVRQMPGKGLEWMGVINPGSGGTNYNE | |
| KFKGQVTISADKSISTAYLQWSSLKASDTAMYYCARWRGEGYYAYFDVWGQGTTVTVSSAST | |
| KGPSVFPLAPSSKSTSGGTAALGCLVKDYFPEPVTVSWNSGALTSGVHTFPAVLQSSGLYSL | |
| SSVVTVPSSSLGTQTYICNVNHKPSNTKVDKRVEPKSCDKTHTCPPCPAPEAAGGPSVFLFP | |
| PKPKDTLMISRTPEVTCVVVDVSHEDPEVKFNWYVDGVEVHNAKTKPREEQYNSTYRVVSVL | |
| TVLHQDWLNGKEYKCKVSNKALAAPIEKTISKAKGQPREPQVYTLPPSREEMTKNQVSLYCL | |
| VKGFYPSDIAVEWESNGQPENNYKTTPPVLDSDGSFFLYSKLTVDKSRWQQGNVFSCSVMHE | |
| ALHNHYTQKSLSLSPGK | |
| SEQ ID NO 091: 21A08Ap3 VL (CDRs) | |
| RSVLTQPPSASGTPGQRVSISCSGASSNIGSNAVFWYQQLPGTAPKLLIYSNNQRPSGVPDR | |
| FSGSKSGTSASLAISGLRSEDEADYYCAAWDDSLSIWVFGGGTKLTVL | |
| SEQ ID NO 092: PXU588 HC: 21A08A p2 VH (CDR)-CLK-Hinge- | |
| CH2-CH3 | |
| EVQLLESGGGLVQPGGSLRLSCAASGFTFSINAMTWVRQAPGKGLEWVSTISGSGFSTYYAD | |
| SLKGRFTISRDNSKNTLYLQMNSLRAEDTAVYYCAKEVYGDYWGQGTLVTVSSASVAAPSVF | |
| IFPPSDEQLKSGTASVVCLLNNFYPREAKVQWKVDNALQSGNSQESVTEQDSKDSTYSLSST | |
| LTLSKADYEKHKVYACEVTHQGLSSPVTKSFNRGECDKTHTCPPCPAPEAAGGPSVFLFPPK | |
| PKDTLMISRTPEVTCVVVDVSHEDPEVKFNWYVDGVEVHNAKTKPREEQYNSTYRVVSVLTV | |
| LHQDWLNGKEYKCKVSNKALAAPIEKTISKAKGQPREPQVYTLPPSREEMTKNQVSLTCLVK | |
| GFYPSDIAVEWESNGQPENNYKTTPPVLDSDGSFFLTSKLTVDKSRWQQGNVESCSVMHEAL | |
| HNHYTQKSLSLSPGK | |
| SEQ ID NO 093: PXU588 21A08A p2 VL (CDR)-CH1 | |
| RSVLTQPPSASGTPGQRVSISCSGASSNIGSSSVFWYQQLPGTAPKLLIYSNNQRPSGVPDR | |
| FSGSKSGTSASLAISGLRSEDEADYYCAAWDDSLSIWVFGGGTKLTVLSSASTKGPSVFPLA | |
| PSSKSTSGGTAALGCLVKDYFPEPVTVSWNSGALTSGVHTFPAVLQSSGLYSLSSVVTVPSS | |
| SLGTQTYICNVNHKPSNTKVDKRVEPKSC | |
| SEQ ID NO 094 IgG scFv N-terminal LTJ498 HC: VH (CDR)-constant | |
| EVQLLESGGGLVQPGGSLRLSCAASGFTFSINAMTWVRQAPGKGLEWVSTISGSGESTYYAD | |
| SLKGRFTISRDNSKNTLYLQMNSLRAEDTAVYYCAKEVYGDYWGQGTLVTVSSASTKGPSVF | |
| PLAPSSKSTSGGTAALGCLVKDYFPEPVTVSWNSGALTSGVHTFPAVLQSSGLYSLSSVVTV | |
| PSSSLGTQTYICNVNHKPSNTKVDKRVEPKSCDKTHTCPPCPAPEAAGGPSVFLFPPKPKDT | |
| LMISRTPEVTCVVVDVSHEDPEVKFNWYVDGVEVHNAKTKPREEQYNSTYRVVSVLTVLHQD | |
| WLNGKEYKCKVSNKALAAPIEKTISKAKGQPREPQVYTLPPSREEMTKNQVSLTCLVKGFYP | |
| SDIAVEWESNGQPENNYKTTPPVLDSDGSFFLTSKLTVDKSRWQQGNVFSCSVMHEALHNHY | |
| TQKSLSLSPGK | |
| SEQ ID NO 095 IgG scFv N-terminal LTJ498 LC: VL (CDR), | |
| constant | |
| RSVLTQPPSASGTPGQRVSISCSGASSNIGSQSVFWYQQLPGTAPKLLIYSNNQRPSGVPDR | |
| FSGSKSGTSASLAISGLRSEDEADYYCAAWDDSLSIWVFGGGTKLTVLGQPKAAPSVTLFPP | |
| SSEELQANKATLVCLISDFYPGAVTVAWKADSSPVKAGVETTTPSKQSNNKYAASSYLSLTP | |
| EQWKSHRSYSCQVTHEGSTVEKTVAPTECS | |
| 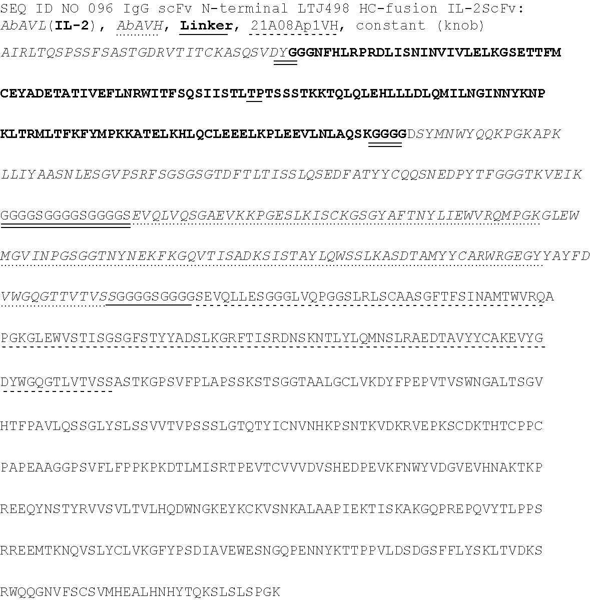 | |
| SEQ ID NO 097 IgG scFv N-terminal JLI141 HC: VH (CDR), | |
| constant | |
| EVQLLESGGGLVQPGGSLRLSCAASGFTFSINAMTWVRQAPGKGLEWVSTISGSGFSTYYAD | |
| SLKGRFTISRDNSKNTLYLQMNSLRAEDTAVYYCAKEVYGDYWGQGTLVTVSSASTKGPSVF | |
| PLAPSSKSTSGGTAALGCLVKDYFPEPVTVSWNSGALTSGVHTFPAVLQSSGLYSLSSVVTV | |
| PSSSLGTQTYICNVNHKPSNTKVDKRVEPKSCDKTHTCPPCPAPEAAGGPSVFLFPPKPKDT | |
| LMISRTPEVTCVVVDVSHEDPEVKFNWYVDGVEVHNAKTKPREEQYNSTYRVVSVLTVLHQD | |
| WLNGKEYKCKVSNKALAAPIEKTISKAKGQPREPQVYTLPPSREEMTKNQVSLTCLVKGFYP | |
| SDIAVEWESNGQPENNYKTTPPVLDSDGSFFLTSKLTVDKSRWQQGNVFSCSVMHEALHNHY | |
| TQKSLSLSPGK | |
| SEQ ID NO 098 IgG scFv N-terminal JLI141 LC: VL (CDR), | |
| constant | |
| RSVLTQPPSASGTPGQRVSISCSGASSNIGSSSVFWYQQLPGTAPKLLIYSNNQRPSGVPDR | |
| FSGSKSGTSASLAISGLRSEDEADYYCAAWDDSLSIWVFGGGTKLTVLGQPKAAPSVTLFPP | |
| SSEELQANKATLVCLISDFYPGAVTVAWKADSSPVKAGVETTTPSKQSNNKYAASSYLSLTP | |
| EQWKSHRSYSCQVTHEGSTVEKTVAPTECS | |
| 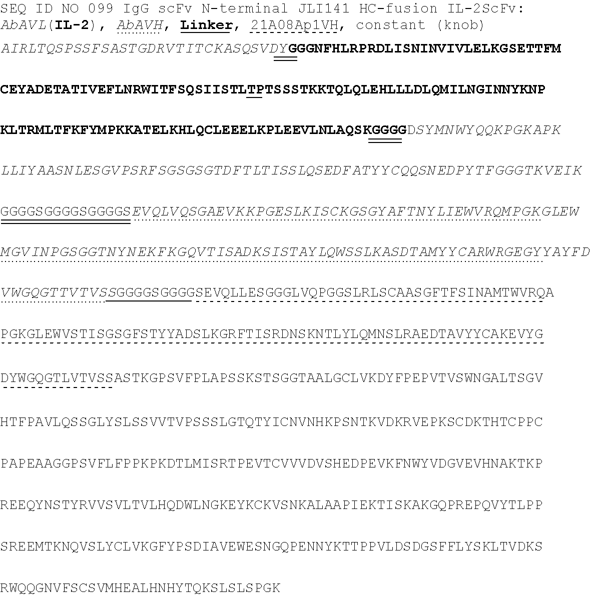 | |
| SEQ ID NO 100 IgG k/I XWY176 QTY065 HC: VH(CDR) constant | |
| EVQLVQSGAEVKKPGESLKISCKGSGYAFTNYLIEWVRQMPGKGLEWMGVINPGSGGTNYNE | |
| KFKGQVTISADKSISTAYLQWSSLKASDTAMYYCARWRGEGYYAYFDVWGQGTTVTVSSAST | |
| KGPSVFPLAPSSKSTSGGTAALGCLVKDYFPEPVTVSWNSGALTSGVHTFPAVLQSSGLYSL | |
| SSVVTVPSSSLGTQTYICNVNHKPSNTKVDKRVEPKSCDKTHTCPPCPAPEAAGGPSVFLFP | |
| PKPKDTLMISRTPEVTCVVVDVSHEDPEVKFNWYVDGVEVHNAKTKPREEQYNSTYRVVSVL | |
| TVLHQDWLNGKEYKCKVSNKALAAPIEKTISKAKGQPREPQVYTLPPSREEMTKNQVSLYCL | |
| VKGFYPSDIAVEWESNGQPENNYKTTPPVLDSDGSFFLYSKLTVDKSRWQQGNVFSCSVMHE | |
| ALHNHYTQKSLSLSPGK | |
| 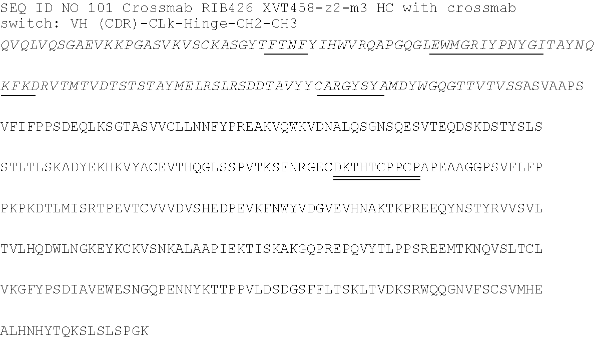 | |
| SEQ ID NO 102: Crossmab RIB426 XVT458-z2-m3 LC with crossmab | |
| switch: VL(CDR)-CH1 | |
| DIQMTQSPSSVSASVGDRVTITCSASQGISGDLNWYQQKPGKAPKLLIYHTSQRHSDVPSRF | |
| SGSGSGTDFTLTISSLQPEDFATYYCQGYSKDLLTFGGGTKLEIKSSASTKGPSVFPLAPSS | |
| KSTSGGTAALGCLVKDYFPEPVTVSWNSGALTSGVHTFPAVLQSSGLYSLSSVVTVPSSSLG | |
| TQTYICNVNHKPSNTKVDKRVEPKSC | |
| 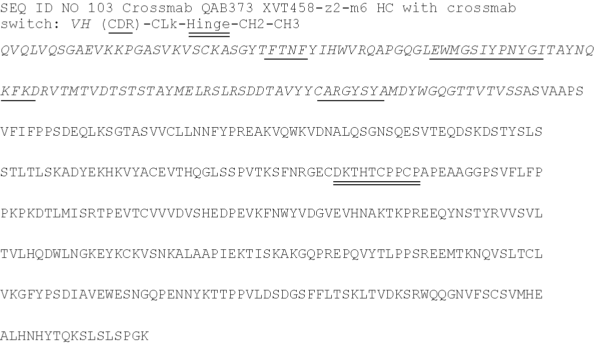 | |
| SEQ ID NO 104: Crossmab QAB373 XVT458-z2-m6 LC with crossmab | |
| switch: VL(CDR)-CH1 | |
| DIQMTQSPSSVSASVGDRVTITCSASQGISGDLNWYQQKPGKAPKLLIYHTSQLHSDVPSRF | |
| SGSGSGTDFTLTISSLQPEDFATYYCQGYSKDLLTFGGGTKLEIKSSASTKGPSVFPLAPSS | |
| KSTSGGTAALGCLVKDYFPEPVTVSWNSGALTSGVHTFPAVLQSSGLYSLSSVVTVPSSSLG | |
| TQTYICNVNHKPSNTKVDKRVEPKSC | |
| SEQ ID NO 105 IgG scFv N-terminal TMU471 HC: VH(CDR)-constant | |
| QVQLVQSGAEVKKPGASVKVSCKASGYTFTNFYIHWVRQAPGQGLEWMGRIYPNYGITAYNQ | |
| KFKDRVTMTVDTSTSTAYMELRSLRSDDTAVYYCARGYSYAMDYWGQGTTVTVSSASTKGPS | |
| VFPLAPSSKSTSGGTAALGCLVKDYFPEPVTVSWNSGALTSGVHTFPAVLQSSGLYSLSSVV | |
| TVPSSSLGTQTYICNVNHKPSNTKVDKRVEPKSCDKTHTCPPCPAPEAAGGPSVFLFPPKPK | |
| DTLMISRTPEVTCVVVDVSHEDPEVKFNWYVDGVEVHNAKTKPREEQYNSTYRVVSVLTVLH | |
| QDWLNGKEYKCKVSNKALAAPIEKTISKAKGQPREPQVYTLPPSREEMTKNQVSLTCLVKGF | |
| YPSDIAVEWESNGQPENNYKTTPPVLDSDGSFFLTSKLTVDKSRWQQGNVFSCSVMHEALHN | |
| HYTQKSLSLSPGK | |
| SEQ ID NO 106 IgG scFv N-terminal TMU471 LC: VL(CDR)-CL(kappa) | |
| DIQMTQSPSSVSASVGDRVTITCSASQGISGDLNWYQQKPGKAPKLLIYHTSQRHSDVPSRF | |
| SGSGSGTDFTLTISSLQPEDFATYYCQGYSKDLLTFGGGTKLEIKRTVAAPSVFIFPPSDEQ | |
| LKSGTASVVCLLNNFYPREAKVQWKVDNALQSGNSQESVTEQDSKDSTYSLSSTLTLSKADY | |
| EKHKVYACEVTHQGLSSPVTKSENRGEC | |
| 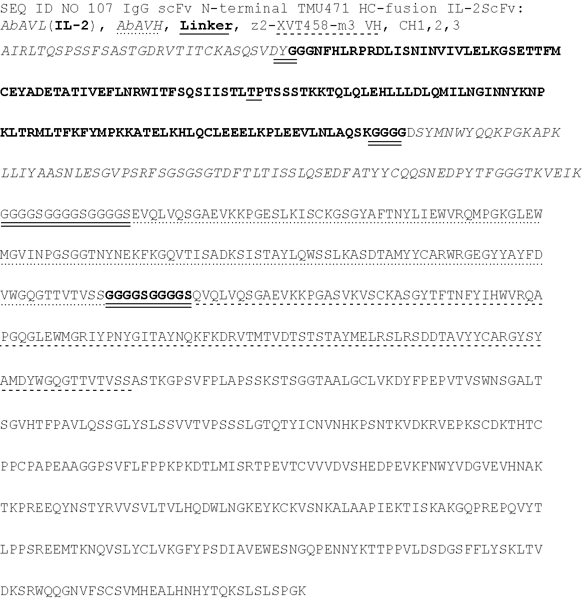 | |
| SEQ ID NO 108 IgG scFv N-terminal MDS446 HC: VH(CDR)-constant | |
| QVQLVQSGAEVKKPGASVKVSCKASGYTFTNFYIHWVRQAPGQGLEWMGSIYPNYGITAYNQ | |
| KFKDRVTMTVDTSTSTAYMELRSLRSDDTAVYYCARGYSYAMDYWGQGTTVTVSSASTKGPS | |
| VFPLAPSSKSTSGGTAALGCLVKDYFPEPVTVSWNSGALTSGVHTFPAVLQSSGLYSLSSVV | |
| TVPSSSLGTQTYICNVNHKPSNTKVDKRVEPKSCDKTHTCPPCPAPEAAGGPSVFLFPPKPK | |
| DTLMISRTPEVTCVVVDVSHEDPEVKFNWYVDGVEVHNAKTKPREEQYNSTYRVVSVLTVLH | |
| QDWLNGKEYKCKVSNKALAAPIEKTISKAKGQPREPQVYTLPPSREEMTKNQVSLTCLVKGF | |
| YPSDIAVEWESNGQPENNYKTTPPVLDSDGSFFLTSKLTVDKSRWQQGNVFSCSVMHEALHN | |
| HYTQKSLSLSPGK | |
| SEQ ID NO 109 IgG scFv N-terminal MDS446LC: VL(CDR)-CL(kappa) | |
| DIQMTQSPSSVSASVGDRVTITCSASQGISGDLNWYQQKPGKAPKLLIYHTSQLHSDVPSRE | |
| SGSGSGTDETLTISSLQPEDFATYYCQGYSKDLLTFGGGTKLEIKRTVAAPSVFIFPPSDEQ | |
| LKSGTASVVCLLNNFYPREAKVQWKVDNALQSGNSQESVTEQDSKDSTYSLSSTLTLSKADY | |
| EKHKVYACEVTHQGLSSPVTKSENRGEC | |
| 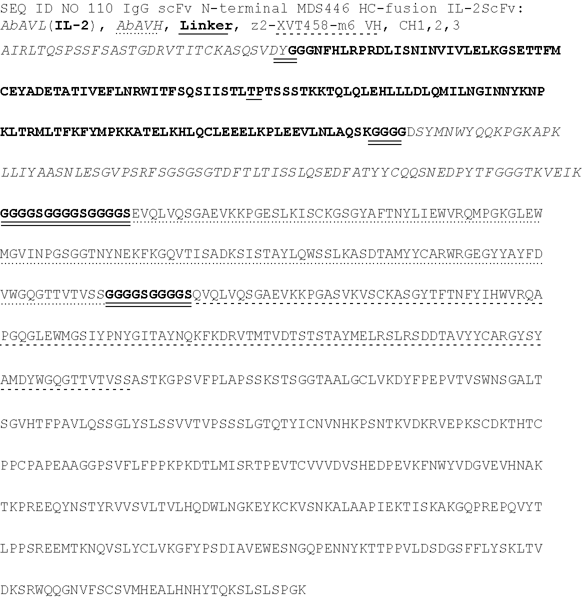 | |
| SEQ ID NO 111 21A08Ap1 HC (NZA596): VH(CDR), constant | |
| EVQLLESGGGLVQPGGSLRLSCAASGFTFSINAMTWVRQAPGKGLEWVSTISGSGFSTYYAD | |
| SLKGRFTISRDNSKNTLYLQMNSLRAEDTAVYYCAKEVYGDYWGQGTLVTVSSASTKGPSVF | |
| PLAPSSKSTSGGTAALGCLVKDYFPEPVTVSWNSGALTSGVHTFPAVLQSSGLYSLSSVVTV | |
| PSSSLGTQTYICNVNHKPSNTKVDKRVEPKSCDKTHTCPPCPAPEAAGGPSVFLFPPKPKDT | |
| LMISRTPEVTCVVVDVSHEDPEVKFNWYVDGVEVHNAKTKPREEQYNSTYRVVSVLTVLHQD | |
| WLNGKEYKCKVSNKALAAPIEKTISKAKGQPREPQVCTLPPSREEMTKNQVSLSCAVKGFYP | |
| SDIAVEWESNGQPENNYKTTPPVLDSDGSFFLVSKLTVDKSRWQQGNVFSCSVMHEALHNHY | |
| TQKSLSLSPGK | |
| SEQ ID NO 112 QTY065 HC (NZA596): VH(CDR), constant | |
| EVQLVQSGAEVKKPGESLKISCKGSGYAFTNYLIEWVRQMPGKGLEWMGVINPGSGGTNYNE | |
| KFKGQVTISADKSISTAYLQWSSLKASDTAMYYCARWRGEGYYAYFDVWGQGTTVTVSSAST | |
| KGPSVFPLAPSSKSTSGGTAALGCLVKDYFPEPVTVSWNSGALTSGVHTFPAVLQSSGLYSL | |
| SSVVTVPSSSLGTQTYICNVNHKPSNTKVDKRVEPKSCDKTHTCPPCPAPEAAGGPSVFLFP | |
| PKPKDTLMISRTPEVTCVVVDVSHEDPEVKFNWYVDGVEVHNAKTKPREEQYNSTYRVVSVL | |
| TVLHQDWLNGKEYKCKVSNKALAAPIEKTISKAKGQPREPQVYTLPPCREEMTKNQVSLWCL | |
| VKGFYPSDIAVEWESNGQPENNYKTTPPVLDSDGSFFLYSKLTVDKSRWQQGNVFSCSVMHE | |
| ALHNHYTQKSLSLSPGK | |
| SEQ ID NO 113 XVT-z2-m3 HC (KTX917): VH(CDR)-constant | |
| QVQLVQSGAEVKKPGASVKVSCKASGYTFTNFYIHWVRQAPGQGLEWMGRIYPNYGITAYNQ | |
| KFKDRVTMTVDTSTSTAYMELRSLRSDDTAVYYCARGYSYAMDYWGQGTTVTVSSASTKGPS | |
| VFPLAPSSKSTSGGTAALGCLVKDYFPEPVTVSWNSGALTSGVHTFPAVLQSSGLYSLSSVV | |
| TVPSSSLGTQTYICNVNHKPSNTKVDKRVEPKSCDKTHTCPPCPAPEAAGGPSVFLFPPKPK | |
| DTLMISRTPEVTCVVVDVSHEDPEVKFNWYVDGVEVHNAKTKPREEQYNSTYRVVSVLTVLH | |
| QDWLNGKEYKCKVSNKALAAPIEKTISKAKGQPREPQVCTLPPSREEMTKNQVSLSCAVKGF | |
| YPSDIAVEWESNGQPENNYKTTPPVLDSDGSFFLVSKLTVDKSRWQQGNVFSCSVMHEALHN | |
| HYTQKSLSLSPGK | |
| 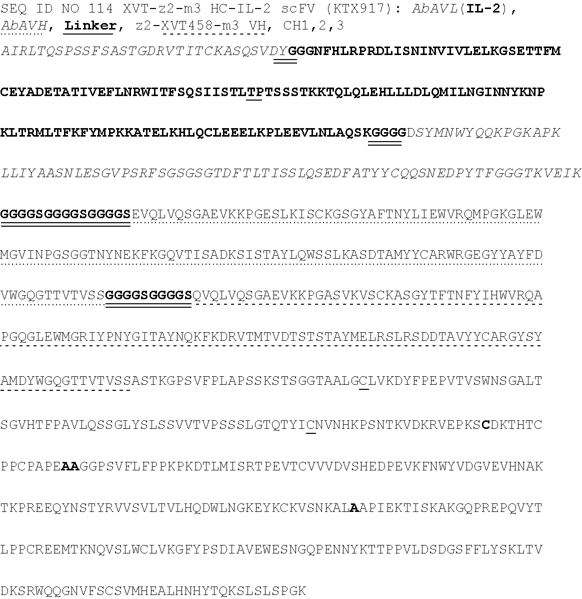 | |
| SEQ ID NO 115: XVT-z2-m6 HC (CIT348) | |
| QVQLVQSGAEVKKPGASVKVSCKASGYTFTNFYIHWVRQAPGQGLEWMGSIYPNYGITAYNQ | |
| KFKDRVTMTVDTSTSTAYMELRSLRSDDTAVYYCARGYSYAMDYWGQGTTVTVSSASTKGPS | |
| VFPLAPSSKSTSGGTAALGCLVKDYFPEPVTVSWNSGALTSGVHTFPAVLQSSGLYSLSSVV | |
| TVPSSSLGTQTYICNVNHKPSNTKVDKRVEPKSCDKTHTCPPCPAPEAAGGPSVFLFPPKPK | |
| DTLMISRTPEVTCVVVDVSHEDPEVKFNWYVDGVEVHNAKTKPREEQYNSTYRVVSVLTVLH | |
| QDWLNGKEYKCKVSNKALAAPIEKTISKAKGQPREPQVCTLPPSREEMTKNQVSLSCAVKGF | |
| YPSDIAVEWESNGQPENNYKTTPPVLDSDGSFFLVSKLTVDKSRWQQGNVFSCSVMHEALHN | |
| HYTQKSLSLSPGK | |
| 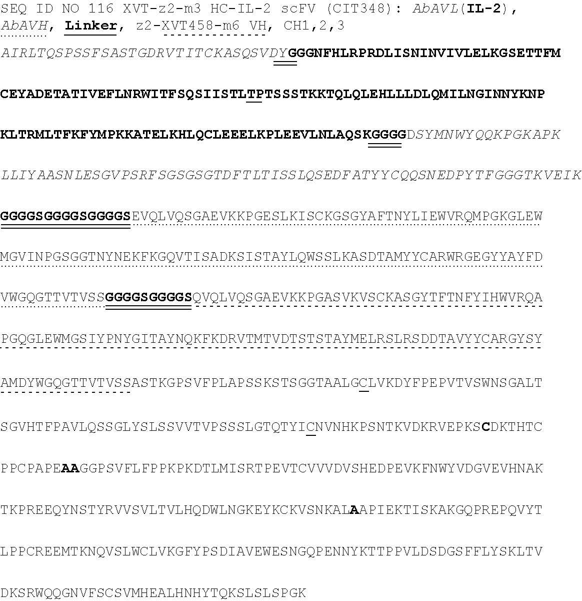 | |
| 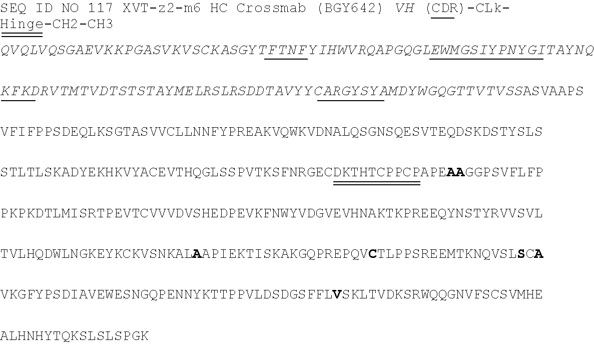 | |
| SEQ ID NO 118: HCDR1 | GFTFSINAMT |
| SEQ ID NO 119: HCDR2 | TISGSGFSTYYADSLKGR |
| SEQ ID NO 120: HCDR3 | EVYGDY |
| SEQ ID NO 121: LCDR1 | SGX¹SSNIGSX²X³VF wherein X1 is N, S, |
| | Q, or A, and where X²X³ is NS, QS, SS, |
| | or NA |
| SEQ ID NO 122: LCDR2 | SNNQRPS |
| SEQ ID NO 123: LCDR3 | AAWDDSLSIWVF |
| SEQ ID NO 124: LCDR1 | SGASSNIGSQSVF |
| SEQ ID NO 125: LCDR1 | SGASSNIGSSSVF |
| SEQ ID NO 126: LCDR1 | SGASSNIGSNAVF |
| SEQ ID NO 127: HCDR1 | NFYIH |
| SEQ ID NO 128: HCDR2 | RIYPNYGITAYNQKFKD |
| SEQ ID NO 129: HCDR3 | GYSYAMDY |
| SEQ ID NO 130: LCDR1 | SASQGISGDLN |
| SEQ ID NO 131: LCDR2 | HTSQRHS |
| SEQ ID NO 132: LCDR3 | QGYSKDLLT |
| SEQ ID NO 133: HCDR2 | SIYPNYGITAYNQKFKD |
| SEQ ID NO 134: LCDR2 | HTSQLHS |
| SEQ ID NO 135: HCDR1 | GFTFSISAMT |
| SEQ ID NO 136: HCDR2 | TISGSGGSTYYSDSVKG |
| SEQ ID NO 137: LCDR1 | SGSSSNIGSNS |
| SEQ ID NO 138: HCDR1 | GFTESTYAMS |
| SEQ ID NO 139: HCDR2 | TISGTGYSTYFADSVKG |
| SEQ ID NO 140: LCDR1 | SGRSSNIGSNS |
| SEQ ID NO 141: LCDR3 | AAWDDSLSGWV |
| SEQ ID NO 142: HCDR1 | GFTFSSYS |
| SEQ ID NO 143: HCDR2 | FISSSSPTLYYADSVKG |
| SEQ ID NO 144: HCDR3 | ARQGLTPEDY |
| SEQ ID NO 145: LCDR1 | RASQSVSSYLA |
| SEQ ID NO 146: LCDR2 | GASTRAT |
| SEQ ID NO 147: LCDR3 | QQYNNWPYT |
| SEQ ID NO 148: HCDR1 | GFTFSNYGMH |
| SEQ ID NO 149: HCDR2 | VIWYDGSKKYYADSVKG |
| SEQ ID NO 150: HCDR3 | NSGH |
| SEQ ID NO 151: LCDR1 | RSSQSLLDSDDGNTYLD |
| SEQ ID NO 152: LCDR2 | TLSYRAS |
| SEQ ID NO 153: LCDR3 | MQCIEFPHT |
| SEQ ID NO 154: HCDR1 | GFTFSSYSMN |
| SEQ ID NO 155: HCDR2 | YITSSSNTMYYADSVKG |
| SEQ ID NO 156: HCDR3 | IVGAPFDY |
| SEQ ID NO 157: LCDR1 | RASQSVSSSLA |
| SEQ ID NO 158: LCDR3 | QQYNNWPFT |
| SEQ ID NO 159: HCDR1 | GYTFTSYFMH |
| SEQ ID NO 160: HCDR2 | LINPDGGNTDYAQKFQG |
| SEQ ID NO 161: HCDR3 | DGNYYDSSGYYYPDAFDI |
| SEQ ID NO 162: LCDR1 | QGDSLRRFYAS |
| SEQ ID NO 163: LCDR2 | GKDNRPS |
| SEQ ID NO 164: LCDR3 | NSRDSSGTHVV |
| SEQ ID NO 165: HCDR2 | XIYPNYGITAYNQKFKD wherein X is R, |
| | or S |
| SEQ ID NO 166: LCDR2 | HTSQXHS wherein X is L or R |

SEQ ID NO 167: QTY065 LC variable region (CDR), circularly
permuted IL2 embedded
AIRLTQSPSSFSASTGDRVTITCKASQSVDYGGGNFHLRPRDLISNINVIVLELKGSETTEM
CEYADETATIVEFLNRWITFSQSIISTLTPTSSSTKKTQLQLEHLLLDLQMILNGINNYKNP
KLTRMLTFKFYMPKKATELKHLQCLEEELKPLEEVLNLAQSKGGGGDSYMNWYQQKPGKAPK
LLIYAASNLESGVPSRFSGSGSGTDFTLTISSLQSEDFATYYCQQSNEDPYTFGGGTKVEIK

1. An immunoconjugate comprising
i. an anti-PD1 antigen binding domain, comprising
an antibody heavy chain variable domain polypeptide (PD1-VH), and
an antibody light chain variable domain polypeptide (PD1-VL);
ii. an anti-IL-2 antigen binding domain, comprising
an antibody heavy chain variable domain polypeptide (IL2-VH) having from N′ to C′ terminus the following domains: a first framework region (IL2VH₁), a first complementarity determining region (IL2HCDR1), a second framework region (IL2VH₂), a second complementarity determining region (IL2HCDR2), a third framework region (IL2VH₃), a third complementarity determining region (IL2HCDR3), and a fourth framework region (IL2VH₄); and
an antibody light chain variable domain polypeptide (IL2-VL) having from N′ to C′ terminus the following domains: a first framework region (IL2VL₁), a first complementarity determining region (IL2LCDR1), a second framework region (IL2VL₂), a second complementarity determining region (IL2LCDR2), a third framework region (IL2VL₃), a third complementarity determining region (an IL2LCDR3), and a fourth framework region (IL2VL₄); and
iii. an IL-2 polypeptide
wherein the IL-2 polypeptide is covalently linked to the IL2-VL, or the IL2-VH, to provide a single contiguous recombinant polypeptide;
characterized in that
binding of pembrolizumab, or nivolumab to a PD-1+ cell does not significantly inhibit binding of the immunoconjugate to said cell.

2. The immunoconjugate according to claim 1, wherein
the PD1-VH comprises an HCDR1 having the sequence GFTFSINAMT (SEQ ID NO 118), an HCDR2 having the sequence TISGSGFSTYYADSLKGR (SEQ ID NO 119), and an HCDR3 having the sequence EVYGDY (SEQ ID NO 120); and
the PD1-VL comprises an LCDR1 having the sequence SGX¹SSNIGSX²X³VF (SEQ ID NO 121), an LCDR2 having the sequence SNNQRPS (SEQ ID NO 122), and an LCDR3 having the sequence AAWDDSLSIWVF (SEQ ID NO 123);
wherein X¹ is N, S, Q, or A;
and wherein X²X³ is NS, QS, SS, or NA.

3. The immunoconjugate according to claim 2, wherein the PD1-VL comprises an LCDR1 having a sequence selected from the group of:
a.
(SEQ ID NO 124)
SGASSNIGSQSVF;
b.
(SEQ ID NO 125)
SGASSNIGSSSVF;
or
c.
(SEQ ID NO 126)
SGASSNIGSNAVF.

4. The immunoconjugate according to claim 2, wherein:
a. the PD1-VH comprises, or consists of a polypeptide having the sequence SEQ ID NO 085 and the PD1-VL comprises, or consists of a polypeptide having the sequence SEQ ID NO 086; or
b. the PD1-VH comprises, or consists of a polypeptide having the sequence SEQ ID NO 085 and the PD1-VL comprises, or consists of a polypeptide having the sequence SEQ ID NO 087; or
c. the PD1-VH comprises, or consists of a polypeptide having the sequence SEQ ID NO 085 VH and the PD1-VL comprises, or consists of a polypeptide having the sequence SEQ ID NO 91.

5. The immunoconjugate according to claim 1, wherein
the PD1-VH comprises, or consists of a polypeptide at least (≥)
95%, 98%, 99% similar to SEQ ID NO 085, and the PD1-VL comprises, or consists of a polypeptide ≥95%, 98%, 99% similar to SEQ ID NO 086;
the PD1-VH comprises, or consists of a polypeptide a polypeptide ≥95%, 98%, 99% similar to SEQ ID NO 085 and the PD1-VL comprises, or consists of a polypeptide ≥95%, 98%,
99% similar to SEQ ID NO 087;
the PD1-VH comprises, or consists of a polypeptide a polypeptide ≥95%, 98%, 99% similar to SEQ ID NO 085; and
the PD1-VL comprises, or consists of a polypeptide ≥95%, 98%,
99% similar to SEQ ID NO 091;
and wherein the affinity constant (K_{D}) of the immunoconjugate for PD-1 is in the range of 1.0×10⁻⁹ to 1.5×10⁻¹¹ mol/L as measured according to the protocol provided in Example 2.

6. The immunoconjugate according to claim 1, wherein
a. the PD1-VH comprises an HCDR1 having the sequence NFYIH (SEQ ID NO 127), an HCDR2 having the sequence RIYPNYGITAYNQKFKD (SEQ ID NO 128), and an HCDR3 having the sequence GYSYAMDY (SEQ ID NO 129); and the PD1-VL comprises an LCDR1 having the sequence SASQGISGDLN (SEQ ID NO 130), an LCDR2 having the sequence HTSQRHS (SEQ ID NO 131), and an LCDR3 having the sequence QGYSKDLLT (SEQ ID NO 132); or
b. the PD1-VH comprises an HCDR1 having the sequence NFYIH (SEQ ID NO 127), an HCDR2 having the sequence SIYPNYGITAYNQKFKD (SEQ ID NO 133), and an HCDR3 having the sequence GYSYAMDY (SEQ ID NO 129); and the PD1-VL comprises an LCDR1 having the sequence SASQGISGDLN (SEQ ID NO 130), an LCDR2 having the sequence HTSQLHS (SEQ ID NO 134), and an LCDR3 having the sequence QGYSKDLLT (SEQ ID NO 132).

7. The immunoconjugate according to claim 1, wherein the IL-2 polypeptide is a circularly permuted IL-2 (IL2CP) polypeptide;
wherein the IL2CP is ≥98% similar to SEQ ID NO 010 or SEQ ID NO 011; and
the IL2CP N′-terminus and IL2CP C′-terminus are covalently linked, optionally by means of one, or two peptide linkers, to amino acid residues within an IL2LCDR1 domain.

8. The immunoconjugate according to claim 1, wherein the IL2-VH is more than >98%, or 99% identical to SEQ ID NO 043, and the IL2-VL is more than >98%, or 99% identical to SEQ ID NO 042; and
wherein the K_{D} of an antibody characterized by the IL-2VH and the IL2-VL for an IL-2 polypeptide having SEQ ID NO 006 is in the range of 270 nM to 2 nM.

9. The immunoconjugate according to claim 1, wherein the anti-IL-2 antigen binding domain comprises, or consists of, an IL2-VL having sequence SEQ ID NO 042, associated with an IL2-VH selected from SEQ ID NO 046, or 047.

10. The immunoconjugate according to claim 1, wherein the immunoconjugate comprises an IgG Fe portion.

11. The immunoconjugate according to claim 10, wherein the IgG Fc portion is characterized by the presence of one or more modifications to constant regions of the heavy chains to enhance correct heavy chain pairing, particularly a set of know and hole modifications selected from:
knob: S354C, T366W and hole: Y349C, T366S, L368A, Y407V;
knob: T366Y, and hole Y407T;
knob: Y349C, T366W, and hole: S354C, T366S, L368A, Y407V; or
knob: T366W, and Hole: Y407A, T366S, L368A.

12. The immunoconjugate according to claim 1, wherein the immunoconjugate is a heterotetrameric IgG consisting of
a first heterodimer comprising the anti-PD-1 binding domain; and
a second heterodimer comprising the anti-IL2 binding domain.

13. The immunoconjugate according to claim 1, wherein the immunoconjugate comprises, or consists of the polypeptides having the sequences:
a. SEQ ID NO 111, SEQ ID NO 095, SEQ ID NO 112, and SEQ ID NO 052;
b. SEQ ID NO 117, SEQ ID NO 104, SEQ ID NO 112, and SEQ ID NO 091;
c. SEQ ID NO 113, 106, and 114;
d. SEQ ID NO 115, 109, and 116;
e. SEQ ID NO 094, SEQ ID NO 095, SEQ ID NO 100, and SEQ ID NO 052;
f. SEQ ID NO 097, SEQ ID NO 098, SEQ ID NO 100, and SEQ ID NO 052.

14. An isolated nucleic acid encoding the immunoconjugate according to claim 1;
particularly wherein the isolated nucleic acid is comprised in a mammalian expression vector under control of a promoter operable in a mammalian cell.

15. A combination medicament comprising
the immunoconjugate as specified in claim 1, and
an anti-PD-1 antagonist antibody selected from the list consisting of nivolumab, pembrolizumab, dostarlimab, sintilimab, tislelizumab, cemiplimab, cetrelimab, sasanlimab, particularly nivolumab or pembrolizumab.


SEQUENCE LISTING (167 sequences)

SEQ ID NO: 1 - protein, 451 aa, synthetic construct
EVQLVQSGAEVKKPGESLKISCKGSGYAFTNYLIEWVRQMPGKGLEWMGVINPGSGGTNYNEKFKGQVTISADKSISTAYLQWSSLKASDTAMYYCARWRGEGYYAYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 218 aa, synthetic construct
AIRLTQSPSSFSASTGDRVTITCKASQSVDYQGDSYMNWYQQKPGKAPKLLIYAASNLESGVPSRFSGSGSGTDFTLTISSLQSEDFATYYCQQSNEDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 451 aa, synthetic construct
QVQLVESGGGVVQPGRSLRLSCAASGYTFSSYLIEWVRQAPGKGLEWVAVINPGSGGTNYADSVKGRFTISADKSKNTAYLQMNSLRAEDTAVYYCARWRGDGYYAYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 218 aa, synthetic construct
EIVLTQSPATLSVSPGERATLSCRASQSVSYDGDSYMNWYQQKPGQAPRLLIYAASNLASGIPARFSGSGSGTEFTLTISSLQSEDAAVYYCQQSNEDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 6 - protein, 134 aa, synthetic construct
MAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 7 - protein, 133 aa, synthetic construct
APTSSSTKKTQLQLEHLLLDLQMILNGINNYKNPKLTAMLTAKFAMPKKATELKHLQCLEEALKPLEEVLNLAQSKNFHLRPRDLISNINVIVLELKGSETTFMCEYADETATIVEFLNRWITFCQSIISTLT

SEQ ID NO: 8 - protein, 133 aa, synthetic construct
APTSSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFCQSIISTLT

SEQ ID NO: 9 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCQSIISTLT

SEQ ID NO: 10 - protein, 132 aa, synthetic construct
NFHLRPRDLISNINVIVLELKGSETTFMCEYADETATIVEFLNRWITFSQSIISTLTPTSSSTKKTQLQLEHLLLDLQMILNGINNYKNPKLTRMLTFKFYMPKKATELKHLQCLEEELKPLEEVLNLAQSK

SEQ ID NO: 11 - protein, 131 aa, synthetic construct
FHLRPRDLISNINVIVLELKGSETTFMCEYADETATIVEFLNRWITFSQSIISTLTPTSSSTKKTQLQLEHLLLDLQMILNGINNYKNPKLTRMLTFKFYMPKKATELKHLQCLEEELKPLEEVLNLAQSK

SEQ ID NO: 12 - protein, 132 aa, synthetic construct
FYMPKKATELKHLQCLEEELKPLEEVLNLAQSKNFHLRPRDLISNINVIVLELKGSETTFMCEYADETATIVEFLNRWITFSQSIISTLTPTSSSTKKTQLQLEHLLLDLQMILNGINNYKNPKLTRMLTFK

SEQ ID NO: 13 - protein, 132 aa, synthetic construct
ATIVEFLNRWITFCQSIISTLTPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADET

SEQ ID NO: 14-16 - skipped (3 entries)

SEQ ID NO: 17 - protein, 4 aa, synthetic construct
GGGG

SEQ ID NO: 18 - protein, 5 aa, synthetic construct
GGGGS

SEQ ID NO: 19 - protein, 6 aa, synthetic construct
GGGGSG

SEQ ID NO: 20 - protein, 7 aa, synthetic construct
GGGGSGG

SEQ ID NO: 21 - protein, 13 aa, synthetic construct
GGGGSGGGGSGGG

SEQ ID NO: 22 - protein, 14 aa, synthetic construct
GGGGSGGGGSGGGG

SEQ ID NO: 23 - protein, 19 aa, synthetic construct
GGGGSGGGGSGGGGSGGGG

SEQ ID NO: 24 - protein, 20 aa, synthetic construct
GGGGSGGGGSGGGGSGGGGS

SEQ ID NO: 25 - protein, 242 aa, synthetic construct
AIRLTQSPSSFSASTGDRVTITCKASQSVDYNFHLRPRDLISNINVIVLELKGSETTFMCEYADETATIVEFLNRWITFSQSIISTLTPTSSSTKKTQLQLEHLLLDLQMILNGINNYKNPKLTRMLTFKFYMPKKATELKHLQCLEEELKPLEEVLNLAQSKGDSYMNWYQQKPGKAPKLLIYAASNLESGVPSRFSGSGSGTDFTLTISSLQSEDFATYYCQQSNEDPYTFGGGTKVEIK

SEQ ID NO: 26 - protein, 246 aa, synthetic construct
AIRLTQSPSSFSASTGDRVTITCKASQSVDYGGNFHLRPRDLISNINVIVLELKGSETTFMCEYADETATIVEFLNRWITFSQSIISTLTPTSSSTKKTQLQLEHLLLDLQMILNGINNYKNPKLTRMLTFKFYMPKKATELKHLQCLEEELKPLEEVLNLAQSKGGGDSYMNWYQQKPGKAPKLLIYAASNLESGVPSRFSGSGSGTDFTLTISSLQSEDFATYYCQQSNEDPYTFGGGTKVEIK

SEQ ID NO: 27 - protein, 248 aa, synthetic construct
AIRLTQSPSSFSASTGDRVTITCKASQSVDYGGGNFHLRPRDLISNINVIVLELKGSETTFMCEYADETATIVEFLNRWITFSQSIISTLTPTSSSTKKTQLQLEHLLLDLQMILNGINNYKNPKLTRMLTFKFYMPKKATELKHLQCLEEELKPLEEVLNLAQSKGGGGDSYMNWYQQKPGKAPKLLIYAASNLESGVPSRFSGSGSGTDFTLTISSLQSEDFATYYCQQSNEDPYTFGGGTKVEIK

SEQ ID NO: 28 - protein, 250 aa, synthetic construct
AIRLTQSPSSFSASTGDRVTITCKASQSVDYGGGGNFHLRPRDLISNINVIVLELKGSETTFMCEYADETATIVEFLNRWITFSQSIISTLTPTSSSTKKTQLQLEHLLLDLQMILNGINNYKNPKLTRMLTFKFYMPKKATELKHLQCLEEELKPLEEVLNLAQSKGGGGSDSYMNWYQQKPGKAPKLLIYAASNLESGVPSRFSGSGSGTDFTLTISSLQSEDFATYYCQQSNEDPYTFGGGTKVEIK

SEQ ID NO: 29 - protein, 254 aa, synthetic construct
AIRLTQSPSSFSASTGDRVTITCKASQSVDYGGGGSGNFHLRPRDLISNINVIVLELKGSETTFMCEYADETATIVEFLNRWITFSQSIISTLTPTSSSTKKTQLQLEHLLLDLQMILNGINNYKNPKLTRMLTFKFYMPKKATELKHLQCLEEELKPLEEVLNLAQSKGGGGSGGDSYMNWYQQKPGKAPKLLIYAASNLESGVPSRFSGSGSGTDFTLTISSLQSEDFATYYCQQSNEDPYTFGGGTKVEIK

SEQ ID NO: 30 - protein, 268 aa, synthetic construct
AIRLTQSPSSFSASTGDRVTITCKASQSVDYGGGGSGGGGSGGGNFHLRPRDLISNINVIVLELKGSETTFMCEYADETATIVEFLNRWITFSQSIISTLTPTSSSTKKTQLQLEHLLLDLQMILNGINNYKNPKLTRMLTFKFYMPKKATELKHLQCLEEELKPLEEVLNLAQSKGGGGSGGGGSGGGGDSYMNWYQQKPGKAPKLLIYAASNLESGVPSRFSGSGSGTDFTLTISSLQSEDFATYYCQQSNEDPYTFGGGTKVEIK

SEQ ID NO: 31 - protein, 280 aa, synthetic construct
AIRLTQSPSSFSASTGDRVTITCKASQSVDYGGGGSGGGGSGGGGSGGGGNFHLRPRDLISNINVIVLELKGSETTFMCEYADETATIVEFLNRWITFSQSIISTLTPTSSSTKKTQLQLEHLLLDLQMILNGINNYKNPKLTRMLTFKFYMPKKATELKHLQCLEEELKPLEEVLNLAQSKGGGGSGGGGSGGGGSGGGGSDSYMNWYQQKPGKAPKLLIYAASNLESGVPSRFSGSGSGTDFTLTISSLQSEDFATYYCQQSNEDPYTFGGGTKVEIK

SEQ ID NO: 32 - protein, 242 aa, synthetic construct
EIVLTQSPATLSVSPGERATLSCRASQSVSYNFHLRPRDLISNINVIVLELKGSETTFMCEYADETATIVEFLNRWITFSQSIISTLTPTSSSTKKTQLQLEHLLLDLQMILNGINNYKNPKLTRMLTFKFYMPKKATELKHLQCLEEELKPLEEVLNLAQSKGDSYMNWYQQKPGQAPRLLIYAASNLASGIPARFSGSGSGTEFTLTISSLQSEDAAVYYCQQSNEDPYTFGGGTKVEIK

SEQ ID NO: 33 - protein, 246 aa, synthetic construct
EIVLTQSPATLSVSPGERATLSCRASQSVSYGGNFHLRPRDLISNINVIVLELKGSETTFMCEYADETATIVEFLNRWITFSQSIISTLTPTSSSTKKTQLQLEHLLLDLQMILNGINNYKNPKLTRMLTFKFYMPKKATELKHLQCLEEELKPLEEVLNLAQSKGGGDSYMNWYQQKPGQAPRLLIYAASNLASGIPARFSGSGSGTEFTLTISSLQSEDAAVYYCQQSNEDPYTFGGGTKVEIK

SEQ ID NO: 34 - protein, 248 aa, synthetic construct
EIVLTQSPATLSVSPGERATLSCRASQSVSYGGGNFHLRPRDLISNINVIVLELKGSETTFMCEYADETATIVEFLNRWITFSQSIISTLTPTSSSTKKTQLQLEHLLLDLQMILNGINNYKNPKLTRMLTFKFYMPKKATELKHLQCLEEELKPLEEVLNLAQSKGGGGDSYMNWYQQKPGQAPRLLIYAASNLASGIPARFSGSGSGTEFTLTISSLQSEDAAVYYCQQSNEDPYTFGGGTKVEIK

SEQ ID NO: 35 - protein, 254 aa, synthetic construct
EIVLTQSPATLSVSPGERATLSCRASQSVSYGGGGSGNFHLRPRDLISNINVIVLELKGSETTFMCEYADETATIVEFLNRWITFSQSIISTLTPTSSSTKKTQLQLEHLLLDLQMILNGINNYKNPKLTRMLTFKFYMPKKATELKHLQCLEEELKPLEEVLNLAQSKGGGGSGGDSYMNWYQQKPGQAPRLLIYAASNLASGIPARFSGSGSGTEFTLTISSLQSEDAAVYYCQQSNEDPYTFGGGTKVEIK

SEQ ID NO: 36 - protein, 268 aa, synthetic construct
EIVLTQSPATLSVSPGERATLSCRASQSVSYGGGGSGGGGSGGGNFHLRPRDLISNINVIVLELKGSETTFMCEYADETATIVEFLNRWITFSQSIISTLTPTSSSTKKTQLQLEHLLLDLQMILNGINNYKNPKLTRMLTFKFYMPKKATELKHLQCLEEELKPLEEVLNLAQSKGGGGSGGGGSGGGGDSYMNWYQQKPGQAPRLLIYAASNLASGIPARFSGSGSGTEFTLTISSLQSEDAAVYYCQQSNEDPYTFGGGTKVEIK

SEQ ID NO: 37 - protein, 240 aa, synthetic construct
DIQMTQSPSSVSASVGDRVTITCSASQGISGNFHLRPRDLISNINVIVLELKGSETTFMCEYADETATIVEFLNRWITFSQSIISTLTPTSSSTKKTQLQLEHLLLDLQMILNGINNYKNPKLTRMLTFKFYMPKKATELKHLQCLEEELKPLEEVLNLAQSKGDLNWYQQKPGKAPKLLIYHTSSLHSGVPSRFSGSGSGTDFTLTISSLQPEDFATYYCQYYSKDLLTFGGGTKLEIK

SEQ ID NO: 38 - protein, 244 aa, synthetic construct
DIQMTQSPSSVSASVGDRVTITCSASQGISGGGNFHLRPRDLISNINVIVLELKGSETTFMCEYADETATIVEFLNRWITFSQSIISTLTPTSSSTKKTQLQLEHLLLDLQMILNGINNYKNPKLTRMLTFKFYMPKKATELKHLQCLEEELKPLEEVLNLAQSKGGGDLNWYQQKPGKAPKLLIYHTSSLHSGVPSRFSGSGSGTDFTLTISSLQPEDFATYYCQYYSKDLLTFGGGTKLEIK

SEQ ID NO: 39 - protein, 246 aa, synthetic construct
DIQMTQSPSSVSASVGDRVTITCSASQGISGGGGNFHLRPRDLISNINVIVLELKGSETTFMCEYADETATIVEFLNRWITFSQSIISTLTPTSSSTKKTQLQLEHLLLDLQMILNGINNYKNPKLTRMLTFKFYMPKKATELKHLQCLEEELKPLEEVLNLAQSKGGGGDLNWYQQKPGKAPKLLIYHTSSLHSGVPSRFSGSGSGTDFTLTISSLQPEDFATYYCQYYSKDLLTFGGGTKLEIK

SEQ ID NO: 40 - protein, 252 aa, synthetic construct
DIQMTQSPSSVSASVGDRVTITCSASQGISGGGGGSGNFHLRPRDLISNINVIVLELKGSETTFMCEYADETATIVEFLNRWITFSQSIISTLTPTSSSTKKTQLQLEHLLLDLQMILNGINNYKNPKLTRMLTFKFYMPKKATELKHLQCLEEELKPLEEVLNLAQSKGGGGSGGDLNWYQQKPGKAPKLLIYHTSSLHSGVPSRFSGSGSGTDFTLTISSLQPEDFATYYCQYYSKDLLTFGGGTKLEIK

SEQ ID NO: 41 - protein, 266 aa, synthetic construct
DIQMTQSPSSVSASVGDRVTITCSASQGISGGGGGSGGGGSGGGNFHLRPRDLISNINVIVLELKGSETTFMCEYADETATIVEFLNRWITFSQSIISTLTPTSSSTKKTQLQLEHLLLDLQMILNGINNYKNPKLTRMLTFKFYMPKKATELKHLQCLEEELKPLEEVLNLAQSKGGGGSGGGGSGGGGDLNWYQQKPGKAPKLLIYHTSSLHSGVPSRFSGSGSGTDFTLTISSLQPEDFATYYCQYYSKDLLTFGGGTKLEIK

SEQ ID NO: 42 - protein, 111 aa, synthetic construct
AIRLTQSPSSFSASTGDRVTITCKASQSVDYQGDSYMNWYQQKPGKAPKLLIYAASNLESGVPSRFSGSGSGTDFTLTISSLQSEDFATYYCQQSNEDPYTFGGGTKVEIK

SEQ ID NO: 43 - protein, 121 aa, synthetic construct
EVQLVQSGAEVKKPGESLKISCKGSGYAFTNYLIEWVRQMPGKGLEWMGVINPGSGGTNYNEKFKGQVTISADKSISTAYLQWSSLKASDTAMYYCARWRGEGYYAYFDVWGQGTTVTVSS

SEQ ID NO: 44 - protein, 121 aa, synthetic construct
QVQLVESGGGVVQPGRSLRLSCAASGYTFSSYLIEWVRQAPGKGLEWVAVINPGSGGTNYADSVKGRFTISADKSKNTAYLQMNSLRAEDTAVYYCARWRGDGYYAYFDVWGQGTTVTVSS

SEQ ID NO: 45 - protein, 117 aa, synthetic construct
EVQLVQSGAEVKKPGESLKISCKGSGYSFTNFYIHWVRQAPGQRLEWMGSIYPNYGDTAYNQKFKDRFVFSLDTSVSTAYLQISSLKAEDTAVYYCARGYSYAMDYWGQGTTVTVSS

SEQ ID NO: 46 - protein, 258 aa, synthetic construct
EVQLVQSGAEVKKPGESLKISCKGSGYAFTNYLIEWVRQMPGKGLEWMGVINPGSGGTNYNEKFKGQVTISADKSISTAYLQWSSLKASDTAMYYCARWRGEGGGFHLRPRDLISNINVIVLELKGSETTFMCEYADETATIVEFLNRWITFSQSIISTLTPTSSSTKKTQLQLEHLLLDLQMILNGINNYKNPKLTRMLTFKFYMPKKATELKHLQCLEEELKPLEEVLNLAQSKGGGGYYAYFDVWGQGTTVTVSS

SEQ ID NO: 47 - protein, 258 aa, synthetic construct
EVQLVQSGAEVKKPGESLKISCKGSGYAFTNYLIEWVRQMPGKGLEWMGVINPGGGFYMPKKATELKHLQCLEEELKPLEEVLNLAQSKNFHLRPRDLISNINVIVLELKGSETTFMCEYADETATIVEFLNRWITFCQSIISTLTPTSSSTKKTQLQLEHLLLDLQMILNGINNYKNPKLTRMLTFKGGGSGGTNYNEKFKGQVTISADKSISTAYLQWSSLKASDTAMYYCARWRGEGYYAYFDVWGQGTTVTVSS

SEQ ID NO: 48 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 107 aa, synthetic construct [sequence omitted]

SEQ ID NO: 50 - protein, 111 aa, synthetic construct
EIVLTQSPATLSVSPGERATLSCRASQSVSYDGDSYMNWYQQKPGQAPRLLIYAASNLASGIPARFSGSGSGTEFTLTISSLQSEDAAVYYCQQSNEDPYTFGGGTKVEIK

SEQ ID NO: 51 - protein, 451 aa, synthetic construct; identical to SEQ ID NO: 1

SEQ ID NO: 52 - protein, 355 aa, synthetic construct
AIRLTQSPSSFSASTGDRVTITCKASQSVDYGGGNFHLRPRDLISNINVIVLELKGSETTFMCEYADETATIVEFLNRWITFSQSIISTLTPTSSSTKKTQLQLEHLLLDLQMILNGINNYKNPKLTRMLTFKFYMPKKATELKHLQCLEEELKPLEEVLNLAQSKGGGGDSYMNWYQQKPGKAPKLLIYAASNLESGVPSRFSGSGSGTDFTLTISSLQSEDFATYYCQQSNEDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 447 aa, synthetic construct
QVQLVQSGAEVKKPGASVKVSCKASGYTFTNFYIHWVRQAPGQGLEWMGSIYPNYGDTAYNQKFKDRVTMTTDTSTSTAYMELRSLRSDDTAVYYCARGYSYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 214 aa, synthetic construct
DIQMTQSPSSVSASVGDRVTITCSASQGISGDLNWYQQKPGKAPKLLIYHTSSLHSGVPSRFSGSGSGTDFTLTISSLQPEDFATYYCQYYSKDLLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 117 aa, synthetic construct
QVQLVQSGAEVKKPGASVKVSCKASGYTFTNFYIHWVRQAPGQGLEWMGSIYPNYGDTAYNQKFKDRVTMTVDTSTSTAYMELRSLRSDDTAVYYCARGYSYAMDYWGQGTTVTVSS

SEQ ID NO: 56 - protein, 107 aa, synthetic construct
DIQMTQSPSSVSASVGDRVTITCSASQGISGDLNWYQQKPGKAPKLLIYHTSSLHSGVPSRFSGSGSGTDFTLTISSLQPEDFATYYCQYYSKDLLTFGGGTKLEIK

SEQ ID NO: 57 - protein, 117 aa, synthetic construct
QVQLVQSGAEVKKPGASVKVSCKASGYTFTNFYIHWVRQAPGQGLEWMGSIYPNYGFTAYNQKFKDRVTMTVDTSTSTAYMELRSLRSDDTAVYYCARGYSYAMDYWGQGTTVTVSS

SEQ ID NO: 58 - protein, 107 aa, synthetic construct
DIQMTQSPSSVSASVGDRVTITCSASQGIWGDLNWYQQKPGKAPKLLIYHTSQLHSDVPSRFSGSGSGTDFTLTISSLQPEDFATYYCQGYSKDLLTFGGGTKLEIK

SEQ ID NO: 59 - protein, 117 aa, synthetic construct
QVQLVQSGAEVKKPGASVKVSCKASGYTFTNFYIHWVRQAPGQGLEWMGRIYPNYGFTAYNQKFKDRVTMTVDTSTSTAYMELRSLRSDDTAVYYCARGYSYAMDYWGQGTTVTVSS

SEQ ID NO: 60 - protein, 107 aa, synthetic construct
DIQMTQSPSSVSASVGDRVTITCSASQGIYGDLNWYQQKPGKAPKLLIYHTSSLHSGVPSRFSGSGSGTDFTLTISSLQPEDFATYYCQAYSKDLLTFGGGTKLEIK

SEQ ID NO: 61 - protein, 117 aa, synthetic construct
QVQLVQSGAEVKKPGASVKVSCKASGYTFTNFYIHWVRQAPGQGLEWMGRIYPNYGITAYNQKFKDRVTMTVDTSTSTAYMELRSLRSDDTAVYYCARGYSYAMDYWGQGTTVTVSS

SEQ ID NO: 62 - protein, 107 aa, synthetic construct
DIQMTQSPSSVSASVGDRVTITCSASQGISGDLNWYQQKPGKAPKLLIYHTSQRHSDVPSRFSGSGSGTDFTLTISSLQPEDFATYYCQGYSKDLLTFGGGTKLEIK

SEQ ID NO: 63 - protein, 117 aa, synthetic construct; identical to SEQ ID NO: 61

SEQ ID NO: 64 - protein, 107 aa, synthetic construct
DIQMTQSPSSVSASVGDRVTITCSASQGISGDLNWYQQKPGKAPKLLIYHTSSRHSGVPSRFSGSGSGTDFTLTISSLQPEDFATYYCQGYSKDLLTFGGGTKLEIK

SEQ ID NO: 65 - protein, 117 aa, synthetic construct; identical to SEQ ID NO: 61

SEQ ID NO: 66 - protein, 107 aa, synthetic construct
DIQMTQSPSSVSASVGDRVTITCSASQGIYGDLNWYQQKPGKAPKLLIYHTSSRHSGVPSRFSGSGSGTDFTLTISSLQPEDFATYYCQAYSKDLLTFGGGTKLEIK

SEQ ID NO: 67 - protein, 117 aa, synthetic construct
QVQLVQSGAEVKKPGASVKVSCKASGYTFTNFYIHWVRQAPGQGLEWMGSIYPNYGITAYNQKFKDRVTMTVDTSTSTAYMELRSLRSDDTAVYYCARGYSYAMDYWGQGTTVTVSS

SEQ ID NO: 68 - protein, 107 aa, synthetic construct
DIQMTQSPSSVSASVGDRVTITCSASQGISGDLNWYQQKPGKAPKLLIYHTSQLHSDVPSRFSGSGSGTDFTLTISSLQPEDFATYYCQGYSKDLLTFGGGTKLEIK

SEQ ID NO: 69 - protein, 114 aa, synthetic construct
QVQLVQSGAEVKKPGASVKVSCKASGYDFTSHWMHWVRQAPGQGLEWMGAIDPSDSYTNYNQKFKGRVTMTTDTSTSTAYMELRSLRSDDTAVYYCARSPFDYWGQGTTVTVSS

SEQ ID NO: 70 - protein, 107 aa, synthetic construct
DIQMTQSPSSLSASVGDRVTITCKASQDVSTAVAWYQQKPGKAPKLLIYSASYRYTGVPSRFSGSGSGTDFTFTISSLQPEDIATYYCQQHYSFPFTFGQGTKLEIK

SEQ ID NO: 71 - protein, 115 aa, synthetic construct
EVQLLESGGGLVQPGGSLRLSCAASGFTFSINAMTWVRQAPGKGLEWVSTISGSGFSTYYADSLKGRFTISRDNSKNTLYLQMNSLRAEDTAVYYCAKEVYGDYWGQGTLVTVSS

SEQ ID NO: 72 - protein, 110 aa, synthetic construct
RSVLTQPPSASGTPGQRVSISCSGNSSNIGSNSVFWYQQLPGTAPKLLIYSNNQRPSGVPDRFSGSKSGTSASLAISGLRSEDEADYYCAAWDDSLSIWVFGGGTKLTVL

SEQ ID NO: 73 - protein, 115 aa, synthetic construct
EVQLLESGGGLVQPGGSLRLSCAASGFTFSISAMTWVRQAPGKGLEWVSTISGSGGSTYYSDSVKGRFTISRDNSKNTLYLQINSLRADDTAVYYCAKEVYGDYWGQGTLVTVSS

SEQ ID NO: 74 - protein, 110 aa, synthetic construct
QSILTQPPSASGTPGQRVTISCSGSSSNIGSNSVFWYQQLPGTAPKLLIFSNNQRPSGVPDRFSGSKSGTSASLAISGLRSEDEADYCCAAWDDSLSGWIFGGGTKLTVL

SEQ ID NO: 75 - protein, 115 aa, synthetic construct
EVQLLESGGNLVQPGGSLRLSCAASGFTFSTYAMSWVRQAPGKGLEWVSTISGTGYSTYFADSVKGRFTISRDNSKNTLFLQMNSLRAEDTAVYYCAKEVYGDYWGQGTLVTVSS

SEQ ID NO: 76 - protein, 110 aa, synthetic construct
QSVLTQPPSASGTPGQRVTISCSGRSSNIGSNSIFWYQQLPGTAPKLLIYSNNQRPSGVPDRFSGSKSGTSASLAISGLRSEDETDYYCAAWDDSLSGWVFGGGTKLTVL

SEQ ID NO: 77 - protein, 117 aa, synthetic construct
EVLLVESGGGLVQPGGSLRLSCAASGFTFSSYSMNWVRQAPGKGLEWVSFISSSSPTLYYADSVKGRFTISRDNAKNSLYLQMNSLRDEDTAVYYCARQGLTPFDYWGQGTLVTVSS

SEQ ID NO: 78 - protein, 107 aa, synthetic construct
EIVMTQSPATLSVFPGERATLSCRASQSVSSYLAWYQQKPGQAPRLLIYGASTRATGIPARVSGSGSGTEFILTISSLQSEDFAVYYCQQYNNWPYTFGQGTKLEIK

SEQ ID NO: 79 - protein, 113 aa, synthetic construct
QVQLVESGGGVGQPGRSLRLSCAASGFTFSNYGMHWVRQAPGKGLEWVAVIWYDGSKKYYADSVKGRFTISRDNSKNTLYLQMNSLRVEDTAVYYCVGNSGHWGQGTLVTVSS

SEQ ID NO: 80 - protein, 113 aa, synthetic construct
DIVMTQTPLSLPVTPGEPASISCRSSQSLLDSDDGNTYLDWYLQKPGQSPQLLIYTLSYRASGVPDRFSGSGSGTDFTLKISRVEAEDVGVYYCMQCIEFPHTFGGGTKVEIK

SEQ ID NO: 81 - protein, 117 aa, synthetic construct
EVQLVESGGGLVQPGGSLRLSCAASGFTFSSYSMNWVRQAPGKGLQWVSYITSSSNTMYYADSVKGRFTISRDNAKNSLYLQMNSLRDEDTAVYYCASIVGAPFDYWGQGTLVTVSS

SEQ ID NO: 82 - protein, 107 aa, synthetic construct
EIVMTQSPATLSVSPGERATLSCRASQSVSSSLAWYQQKPGQAPRLLIYGASTRATGIPARFSGSGSGTEFTLTISSLQSEDFAAYYCQQYNNWPFTFGPGTKVDIK

SEQ ID NO: 83 - protein, 127 aa, synthetic construct
QVQLVQSGAEVKKPGASVKVSCKASGYTFTSYFMHWVRQAPGQGLEWMGLINPDGGNTDYAQKFQGRVTMTRDTSTSTVYMELSSLRSEDTAVYYCARDGNYYDSSGYYYPDAFDIWGQGTMVTVSS

SEQ ID NO: 84 - protein, 108 aa, synthetic construct
SSELTQDPAVSVALGQTVRITCQGDSLRRFYASWYQQKPGQAPVIVIYGKDNRPSGTPDRFSGSTSGNTASLTITGAQAEDEADYYCNSRDSSGTHVVFGGGTKLTVL

SEQ ID NO: 85 - protein, 115 aa, synthetic construct; identical to SEQ ID NO: 71

SEQ ID NO: 86 - protein, 110 aa, synthetic construct
RSVLTQPPSASGTPGQRVSISCSGASSNIGSQSVFWYQQLPGTAPKLLIYSNNQRPSGVPDRFSGSKSGTSASLAISGLRSEDEADYYCAAWDDSLSIWVFGGGTKLTVL

SEQ ID NO: 87 - protein, 110 aa, synthetic construct
RSVLTQPPSASGTPGQRVSISCSGASSNIGSSSVFWYQQLPGTAPKLLIYSNNQRPSGVPDRFSGSKSGTSASLAISGLRSEDEADYYCAAWDDSLSIWVFGGGTKLTVL

SEQ ID NO: 88 - protein, 449 aa, synthetic construct
EVQLLESGGGLVQPGGSLRLSCAASGFTFSINAMTWVRQAPGKGLEWVSTISGSGFSTYYADSLKGRFTISRDNSKNTLYLQMNSLRAEDTAVYYCAKEVYGDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 89 - protein, 215 aa, synthetic construct
RSVLTQPPSASGTPGQRVSISCSGASSNIGSQSVFWYQQLPGTAPKLLIYSNNQRPSGVPDRFSGSKSGTSASLAISGLRSEDEADYYCAAWDDSLSIWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 90 - protein, 451 aa, synthetic construct
EVQLVQSGAEVKKPGESLKISCKGSGYAFTNYLIEWVRQMPGKGLEWMGVINPGSGGTNYNEKFKGQVTISADKSISTAYLQWSSLKASDTAMYYCARWRGEGYYAYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 110 aa, synthetic construct
RSVLTQPPSASGTPGQRVSISCSGASSNIGSNAVFWYQQLPGTAPKLLIYSNNQRPSGVPDRFSGSKSGTSASLAISGLRSEDEADYYCAAWDDSLSIWVFGGGTKLTVL

SEQ ID NO: 92 - protein, 449 aa, synthetic construct; identical to SEQ ID NO: 88

SEQ ID NO: 93 - protein, 215 aa, synthetic construct
RSVLTQPPSASGTPGQRVSISCSGASSNIGSSSVFWYQQLPGTAPKLLIYSNNQRPSGVPDRFSGSKSGTSASLAISGLRSEDEADYYCAAWDDSLSIWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 94 - protein, 445 aa, synthetic construct
EVQLLESGGGLVQPGGSLRLSCAASGFTFSINAMTWVRQAPGKGLEWVSTISGSGFSTYYADSLKGRFTISRDNSKNTLYLQMNSLRAEDTAVYYCAKEVYGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 95 - protein, 216 aa, synthetic construct
RSVLTQPPSASGTPGQRVSISCSGASSNIGSQSVFWYQQLPGTAPKLLIYSNNQRPSGVPDRFSGSKSGTSASLAISGLRSEDEADYYCAAWDDSLSIWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 96 - protein, 839 aa, synthetic construct
AIRLTQSPSSFSASTGDRVTITCKASQSVDYGGGNFHLRPRDLISNINVIVLELKGSETTFMCEYADETATIVEFLNRWITFSQSIISTLTPTSSSTKKTQLQLEHLLLDLQMILNGINNYKNPKLTRMLTFKFYMPKKATELKHLQCLEEELKPLEEVLNLAQSKGGGGDSYMNWYQQKPGKAPKLLIYAASNLESGVPSRFSGSGSGTDFTLTISSLQSEDFATYYCQQSNEDPYTFGGGTKVEIKGGGGSGGGGSGGGGSEVQLVQSGAEVKKPGESLKISCKGSGYAFTNYLIEWVRQMPGKGLEWMGVINPGSGGTNYNEKFKGQVTISADKSISTAYLQWSSLKASDTAMYYCARWRGEGYYAYFDVWGQGTTVTVSSGGGGSGGGGSEVQLLESGGGLVQPGGSLRLSCAASGFTFSINAMTWVRQAPGKGLEWVSTISGSGFSTYYADSLKGRFTISRDNSKNTLYLQMNSLRAEDTAVYYCAKEVYGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 445 aa, synthetic construct; identical to SEQ ID NO: 94

SEQ ID NO: 98 - protein, 216 aa, synthetic construct
RSVLTQPPSASGTPGQRVSISCSGASSNIGSSSVFWYQQLPGTAPKLLIYSNNQRPSGVPDRFSGSKSGTSASLAISGLRSEDEADYYCAAWDDSLSIWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 99 - protein, 839 aa, synthetic construct; identical to SEQ ID NO: 96

SEQ ID NO: 100 - protein, 451 aa, synthetic construct; identical to SEQ ID NO: 90

SEQ ID NO: 101 - protein, 451 aa, synthetic construct
QVQLVQSGAEVKKPGASVKVSCKASGYTFTNFYIHWVRQAPGQGLEWMGRIYPNYGITAYNQKFKDRVTMTVDTSTSTAYMELRSLRSDDTAVYYCARGYSYAMDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 102 - protein, 212 aa, synthetic construct
DIQMTQSPSSVSASVGDRVTITCSASQGISGDLNWYQQKPGKAPKLLIYHTSQRHSDVPSRFSGSGSGTDFTLTISSLQPEDFATYYCQGYSKDLLTFGGGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 103 - protein, 451 aa, synthetic construct
QVQLVQSGAEVKKPGASVKVSCKASGYTFTNFYIHWVRQAPGQGLEWMGSIYPNYGITAYNQKFKDRVTMTVDTSTSTAYMELRSLRSDDTAVYYCARGYSYAMDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 104 - protein, 212 aa, synthetic construct
DIQMTQSPSSVSASVGDRVTITCSASQGISGDLNWYQQKPGKAPKLLIYHTSQLHSDVPSRFSGSGSGTDFTLTISSLQPEDFATYYCQGYSKDLLTFGGGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 105 - protein, 447 aa, synthetic construct
QVQLVQSGAEVKKPGASVKVSCKASGYTFTNFYIHWVRQAPGQGLEWMGRIYPNYGITAYNQKFKDRVTMTVDTSTSTAYMELRSLRSDDTAVYYCARGYSYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 106 - protein, 214 aa, synthetic construct
DIQMTQSPSSVSASVGDRVTITCSASQGISGDLNWYQQKPGKAPKLLIYHTSQRHSDVPSRFSGSGSGTDFTLTISSLQPEDFATYYCQGYSKDLLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 841 aa, synthetic construct
AIRLTQSPSSFSASTGDRVTITCKASQSVDYGGGNFHLRPRDLISNINVIVLELKGSETTFMCEYADETATIVEFLNRWITFSQSIISTLTPTSSSTKKTQLQLEHLLLDLQMILNGINNYKNPKLTRMLTFKFYMPKKATELKHLQCLEEELKPLEEVLNLAQSKGGGGDSYMNWYQQKPGKAPKLLIYAASNLESGVPSRFSGSGSGTDFTLTISSLQSEDFATYYCQQSNEDPYTFGGGTKVEIKGGGGSGGGGSGGGGSEVQLVQSGAEVKKPGESLKISCKGSGYAFTNYLIEWVRQMPGKGLEWMGVINPGSGGTNYNEKFKGQVTISADKSISTAYLQWSSLKASDTAMYYCARWRGEGYYAYFDVWGQGTTVTVSSGGGGSGGGGSQVQLVQSGAEVKKPGASVKVSCKASGYTFTNFYIHWVRQAPGQGLEWMGRIYPNYGITAYNQKFKDRVTMTVDTSTSTAYMELRSLRSDDTAVYYCARGYSYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 447 aa, synthetic construct
QVQLVQSGAEVKKPGASVKVSCKASGYTFTNFYIHWVRQAPGQGLEWMGSIYPNYGITAYNQKFKDRVTMTVDTSTSTAYMELRSLRSDDTAVYYCARGYSYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 109 - protein, 214 aa, synthetic construct
DIQMTQSPSSVSASVGDRVTITCSASQGISGDLNWYQQKPGKAPKLLIYHTSQLHSDVPSRFSGSGSGTDFTLTISSLQPEDFATYYCQGYSKDLLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 841 aa, synthetic construct
AIRLTQSPSSFSASTGDRVTITCKASQSVDYGGGNFHLRPRDLISNINVIVLELKGSETTFMCEYADETATIVEFLNRWITFSQSIISTLTPTSSSTKKTQLQLEHLLLDLQMILNGINNYKNPKLTRMLTFKFYMPKKATELKHLQCLEEELKPLEEVLNLAQSKGGGGDSYMNWYQQKPGKAPKLLIYAASNLESGVPSRFSGSGSGTDFTLTISSLQSEDFATYYCQQSNEDPYTFGGGTKVEIKGGGGSGGGGSGGGGSEVQLVQSGAEVKKPGESLKISCKGSGYAFTNYLIEWVRQMPGKGLEWMGVINPGSGGTNYNEKFKGQVTISADKSISTAYLQWSSLKASDTAMYYCARWRGEGYYAYFDVWGQGTTVTVSSGGGGSGGGGSQVQLVQSGAEVKKPGASVKVSCKASGYTFTNFYIHWVRQAPGQGLEWMGSIYPNYGITAYNQKFKDRVTMTVDTSTSTAYMELRSLRSDDTAVYYCARGYSYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 445 aa, synthetic construct
EVQLLESGGGLVQPGGSLRLSCAASGFTFSINAMTWVRQAPGKGLEWVSTISGSGFSTYYADSLKGRFTISRDNSKNTLYLQMNSLRAEDTAVYYCAKEVYGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 112 - protein, 451 aa, synthetic construct
EVQLVQSGAEVKKPGESLKISCKGSGYAFTNYLIEWVRQMPGKGLEWMGVINPGSGGTNYNEKFKGQVTISADKSISTAYLQWSSLKASDTAMYYCARWRGEGYYAYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 447 aa, synthetic construct
QVQLVQSGAEVKKPGASVKVSCKASGYTFTNFYIHWVRQAPGQGLEWMGRIYPNYGITAYNQKFKDRVTMTVDTSTSTAYMELRSLRSDDTAVYYCARGYSYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 114 - protein, 841 aa, synthetic construct
AIRLTQSPSSFSASTGDRVTITCKASQSVDYGGGNFHLRPRDLISNINVIVLELKGSETTFMCEYADETATIVEFLNRWITFSQSIISTLTPTSSSTKKTQLQLEHLLLDLQMILNGINNYKNPKLTRMLTFKFYMPKKATELKHLQCLEEELKPLEEVLNLAQSKGGGGDSYMNWYQQKPGKAPKLLIYAASNLESGVPSRFSGSGSGTDFTLTISSLQSEDFATYYCQQSNEDPYTFGGGTKVEIKGGGGSGGGGSGGGGSEVQLVQSGAEVKKPGESLKISCKGSGYAFTNYLIEWVRQMPGKGLEWMGVINPGSGGTNYNEKFKGQVTISADKSISTAYLQWSSLKASDTAMYYCARWRGEGYYAYFDVWGQGTTVTVSSGGGGSGGGGSQVQLVQSGAEVKKPGASVKVSCKASGYTFTNFYIHWVRQAPGQGLEWMGRIYPNYGITAYNQKFKDRVTMTVDTSTSTAYMELRSLRSDDTAVYYCARGYSYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 447 aa, synthetic construct
QVQLVQSGAEVKKPGASVKVSCKASGYTFTNFYIHWVRQAPGQGLEWMGSIYPNYGITAYNQKFKDRVTMTVDTSTSTAYMELRSLRSDDTAVYYCARGYSYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 116 - protein, 841 aa, synthetic construct
AIRLTQSPSSFSASTGDRVTITCKASQSVDYGGGNFHLRPRDLISNINVIVLELKGSETTFMCEYADETATIVEFLNRWITFSQSIISTLTPTSSSTKKTQLQLEHLLLDLQMILNGINNYKNPKLTRMLTFKFYMPKKATELKHLQCLEEELKPLEEVLNLAQSKGGGGDSYMNWYQQKPGKAPKLLIYAASNLESGVPSRFSGSGSGTDFTLTISSLQSEDFATYYCQQSNEDPYTFGGGTKVEIKGGGGSGGGGSGGGGSEVQLVQSGAEVKKPGESLKISCKGSGYAFTNYLIEWVRQMPGKGLEWMGVINPGSGGTNYNEKFKGQVTISADKSISTAYLQWSSLKASDTAMYYCARWRGEGYYAYFDVWGQGTTVTVSSGGGGSGGGGSQVQLVQSGAEVKKPGASVKVSCKASGYTFTNFYIHWVRQAPGQGLEWMGSIYPNYGITAYNQKFKDRVTMTVDTSTSTAYMELRSLRSDDTAVYYCARGYSYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 451 aa, synthetic construct
QVQLVQSGAEVKKPGASVKVSCKASGYTFTNFYIHWVRQAPGQGLEWMGSIYPNYGITAYNQKFKDRVTMTVDTSTSTAYMELRSLRSDDTAVYYCARGYSYAMDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 118 - protein, 10 aa, synthetic construct
GFTFSINAMT

SEQ ID NO: 119 - protein, 18 aa, synthetic construct
TISGSGFSTYYADSLKGR

SEQ ID NO: 120 - protein, 6 aa, synthetic construct
EVYGDY

SEQ ID NO: 121 - protein, 14 aa, synthetic construct
  VARIANT 3: note = X = N, S,Q or A
  REGION 11..12: note = XX = NS, QS, SS or NA
SGXSSNIGSXXXVF

SEQ ID NO: 122 - protein, 7 aa, synthetic construct
SNNQRPS

SEQ ID NO: 123 - protein, 12 aa, synthetic construct
AAWDDSLSIWVF

SEQ ID NO: 124 - protein, 13 aa, synthetic construct
SGASSNIGSQSVF

SEQ ID NO: 125 - protein, 13 aa, synthetic construct
SGASSNIGSSSVF

SEQ ID NO: 126 - protein, 13 aa, synthetic construct
SGASSNIGSNAVF

SEQ ID NO: 127 - protein, 5 aa, synthetic construct
NFYIH

SEQ ID NO: 128 - protein, 17 aa, synthetic construct
RIYPNYGITAYNQKFKD

SEQ ID NO: 129 - protein, 8 aa, synthetic construct
GYSYAMDY

SEQ ID NO: 130 - protein, 11 aa, synthetic construct
SASQGISGDLN

SEQ ID NO: 131 - protein, 7 aa, synthetic construct
HTSQRHS

SEQ ID NO: 132 - protein, 9 aa, synthetic construct
QGYSKDLLT

SEQ ID NO: 133 - protein, 17 aa, synthetic construct
SIYPNYGITAYNQKFKD

SEQ ID NO: 134 - protein, 7 aa, synthetic construct
HTSQLHS

SEQ ID NO: 135 - protein, 10 aa, synthetic construct
GFTFSISAMT

SEQ ID NO: 136 - protein, 17 aa, synthetic construct
TISGSGGSTYYSDSVKG

SEQ ID NO: 137 - protein, 11 aa, synthetic construct
SGSSSNIGSNS

SEQ ID NO: 138 - protein, 10 aa, synthetic construct
GFTFSTYAMS

SEQ ID NO: 139 - protein, 17 aa, synthetic construct
TISGTGYSTYFADSVKG

SEQ ID NO: 140 - protein, 11 aa, synthetic construct
SGRSSNIGSNS

SEQ ID NO: 141 - protein, 11 aa, synthetic construct
AAWDDSLSGWV

SEQ ID NO: 142 - protein, 8 aa, synthetic construct
GFTFSSYS

SEQ ID NO: 143 - protein, 17 aa, synthetic construct
FISSSSPTLYYADSVKG

SEQ ID NO: 144 - protein, 10 aa, synthetic construct
ARQGLTPFDY

SEQ ID NO: 145 - protein, 11 aa, synthetic construct
RASQSVSSYLA

SEQ ID NO: 146 - protein, 7 aa, synthetic construct
GASTRAT

SEQ ID NO: 147 - protein, 9 aa, synthetic construct
QQYNNWPYT

SEQ ID NO: 148 - protein, 10 aa, synthetic construct
GFTFSNYGMH

SEQ ID NO: 149 - protein, 17 aa, synthetic construct
VIWYDGSKKYYADSVKG

SEQ ID NO: 150 - protein, 4 aa, synthetic construct
NSGH

SEQ ID NO: 151 - protein, 17 aa, synthetic construct
RSSQSLLDSDDGNTYLD

SEQ ID NO: 152 - protein, 7 aa, synthetic construct
TLSYRAS

SEQ ID NO: 153 - protein, 9 aa, synthetic construct
MQCIEFPHT

SEQ ID NO: 154 - protein, 10 aa, synthetic construct
GFTFSSYSMN

SEQ ID NO: 155 - protein, 17 aa, synthetic construct
YITSSSNTMYYADSVKG

SEQ ID NO: 156 - protein, 8 aa, synthetic construct
IVGAPFDY

SEQ ID NO: 157 - protein, 11 aa, synthetic construct
RASQSVSSSLA

SEQ ID NO: 158 - protein, 9 aa, synthetic construct
QQYNNWPFT

SEQ ID NO: 159 - protein, 10 aa, synthetic construct
GYTFTSYFMH

SEQ ID NO: 160 - protein, 17 aa, synthetic construct
LINPDGGNTDYAQKFQG

SEQ ID NO: 161 - protein, 18 aa, synthetic construct
DGNYYDSSGYYYPDAFDI

SEQ ID NO: 162 - protein, 11 aa, synthetic construct
QGDSLRRFYAS

SEQ ID NO: 163 - protein, 7 aa, synthetic construct
GKDNRPS

SEQ ID NO: 164 - protein, 11 aa, synthetic construct
NSRDSSGTHVV

SEQ ID NO: 165 - protein, 17 aa, synthetic construct
  VARIANT 1: note = X = R or S
XIYPNYGITAYNQKFKD

SEQ ID NO: 166 - protein, 7 aa, synthetic construct
  VARIANT 5: note = X = L or R
HTSQXHS

SEQ ID NO: 167 - protein, 248 aa, synthetic construct; identical to SEQ ID NO: 27